(19) United States
(12) Patent Application Publication
(10) Pub. No.: US 2016/0089446 A1
(43) Pub. Date: Mar. 31, 2016

(54) Modified Hydrogels

(71) Applicant: ASCENDIS PHARMA A/S, Hellerup (DK)

(72) Inventors: Ulrich HERSEL, Heidelberg (DE); Harald RAU, Dossenheim (DE); Burkhardt LAUFER, Dossenheim (DE); Joachim ZETTLER, Dossenheim (DE); Romy REIMANN, Heidelberg (DE)

(73) Assignee: Ascendis Pharma A/S, Hellerup (DK)

(21) Appl. No.: 14/786,456

(22) PCT Filed: Apr. 16, 2014
(86) PCT No.: PCT/EP2014/057760

§ 371 Date: Oct. 22, 2015

(30) Foreign Application Priority Data
Apr. 22, 2013 (EP) 13164674.7
Oct. 8, 2013 (EP) 13187779.7

(51) Int. Cl. A61K 47/48; A61K 38/20; C08G 65/48; A61K 38/28

(52) U.S. Cl. CPC A61K 47/48215; A61K 47/48784; A61K 38/28; A61K 38/2006; C08G 65/48

(57) ABSTRACT

The present invention relates to a process for the preparation of a hydrogel suitable as carrier in a hydrogel-linked prodrug, to hydrogels obtainable from said process, the use of such hydrogel as a carrier in a hydrogel-linked prodrug and to hydrogel-linked prodrugs comprising a covalently conjugated hydrogel of the present invention. The hydrogel prodrug carrier has a reduced drug loading on the outside of the hydrogel carrier. This is achieved by reducing the number of functional groups of the hydrogel, in particular those at its surface.

### FIELD OF THE INVENTION

The present invention relates to a process for the preparation of a hydrogel suitable as carrier in a hydrogel-linked prodrug, to hydrogels obtainable from said process, the use of such hydrogel as a carrier in a hydrogel-linked prodrug and to hydrogel-linked prodrugs comprising a covalently conjugated hydrogel of the present invention.

### BACKGROUND OF THE INVENTION

Hydrogels are versatile carriers for carrier-linked prodrugs, see for example WO2006003014A2 and WO2011012715A1. As most of the drugs are connected to the inside of the hydrogel, they are protected from modifying and/or degrading enzymes present in a patient's body which extends the time period over which active drugs are released from such prodrugs.

However, part of the drug load of a hydrogel carrier is also connected to the outside of the hydrogel carrier, which in selected cases may potentially be disadvantageous. One disadvantage may be that drug molecules attached to the outside of the hydrogel may be exposed to modifying and/or degrading enzymes present in a patient's body upon administration of the hydrogel-linked prodrug to a patient.

Another disadvantage may be that drugs attached to the outside of the hydrogel may potentially have a certain level of residual activity or immunogenicity which in rare cases may cause undesired effects, such as immune reactions and/or inflammations.

### DETAILED DESCRIPTION OF THE INVENTION

Accordingly, there is a need for hydrogel prodrug carriers which at least partially overcome the above shortcomings.

It is therefore an object of the present invention to overcome at least some of the above-mentioned shortcomings and to provide hydrogel prodrug carriers which have a reduced drug loading on the outside of the hydrogel carrier. This is achieved by reducing the number of functional groups of the hydrogel, in particular those at its surface.

In one aspect, the present invention relates to a process for the preparation of a hydrogel suitable as carrier in a hydrogel-linked prodrug comprising the steps of
(a) providing a hydrogel having groups A^{x0}, wherein groups A^{x0} represent the same or different, preferably same, functional groups;
(b) optionally covalently conjugating a spacer reagent of formula (I)

A^{x1}-SP²-A^{x2} (I),
wherein
SP² is C₁₋₅₀ alkyl, C₂₋₅₀ alkenyl or C₂₋₅₀ alkynyl, which C₁₋₅₀ alkyl, C₂₋₅₀ alkenyl and C₂₋₅₀ alkynyl is optionally interrupted by one or more group(s) selected from the group consisting of —NH—, —N(C₁₋₄alkyl)-, —O—, —S, —C(O)—, —C(O)NH, —C(O)N(C₁₋₄alkyl)-, —O—C(O)—, —S(O)—, —S(O)₂—, 4- to 7-membered heterocyclyl, phenyl and naphthyl;
A^{x1} is a functional group for reaction with A^{x0} of the hydrogel; and
A^{x2} is a functional group;
to A^{x0} of the hydrogel from step (a); and
(c) reacting the hydrogel of step (a) or step (b) with a reagent of formula (II)

A^{x3}-Z (II),
wherein
A^{x3} is a functional group; and
Z is an inert moiety having a molecular weight ranging from 10 Da to 1000 kDa;
such that at most 99 mol-% of A^{x0} or A^{x2} react with A^{x3}.

It was now surprisingly found that such modified hydrogels have a reduced number of functional groups A^{x0} available on the surface of the hydrogel compared to unmodified hydrogels and thus when such hydrogel is used as a carrier for a hydrogel-linked prodrug has fewer biologically active moieties attached to its surface.

Within the present invention the terms are used with the meaning as follows.

As used herein, the term “hydrogel” means a hydrophilic or amphiphilic polymeric network composed of homopolymers or copolymers, which is insoluble due to the presence of covalent chemical crosslinks. The crosslinks provide the network structure and physical integrity. Hydrogels exhibit a thermodynamic compatibility with water which allows them to swell in aqueous media.

As used herein, the term “reagent” means a chemical compound which comprises at least one functional group for reaction with the functional group of another reagent or moiety.

As used herein, the term “backbone reagent” means a reagent, which is suitable as a starting material for forming hydrogels. As used herein, a backbone reagent preferably does not comprise biodegradable linkages. A backbone reagent may comprise a “branching core” which refers to an atom or moiety to which more than one other moiety is attached.

As used herein, the term “crosslinker reagent” means a linear or branched reagent, which is suitable as a starting material for crosslinking backbone reagents. Preferably, the crosslinker reagent is a linear chemical compound. A crosslinker reagent comprises at least one biodegradable linkage.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety.

Accordingly, the phrase “in bound form” is used to refer to the corresponding moiety of a reagent, i.e. “lysine in bound form” refers to a lysine moiety which lacks one or more atom(s) of the lysine reagent and is part of a molecule.

The term “drug” means any substance which can effect one or more physical or biochemical properties of a biological organism, including but not limited to viruses, bacteria, fungi, plants, animals, and humans. In particular, as used herein, the term includes any substance intended for diagnosis, cure, mitigation, treatment, or prevention of disease in organisms, in particular humans or animals, or to otherwise enhance physical or mental well-being of organisms, in particular humans or animals.

The term “biologically active moiety” refers to the moiety which results after covalently conjugating a drug to one or more other moieties wherein one or more functional groups of the drug were conjugated to functional groups of said one or more other moieties which subsequently form linkages.

The term “spacer moiety” as used herein refers to any moiety suitable for connecting two moieties and suitable spacer moieties are known to the person skilled in the art.

As used herein, the term “functional group” means a group of atoms which can react with other functional groups. Functional groups include but are not limited to the following groups: carboxylic acid (—(C═O)OH), primary or secondary amine (—NH₂, —NH—), maleimide, thiol (—SH), sulfonic acid (—(O═S═O)OH), carbonate, carbamate (—O(C═O)N<), hydroxy (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, vinyl sulfone, vinyl ketone, diazoalkane, oxirane, and aziridine.

As used herein, the term “activated functional group” means a functional group, which is connected to an activating group, i.e. a functional group was reacted with an activating reagent. Preferred activated functional groups include but are not limited to activated ester groups, activated carbamate groups, activated carbonate groups and activated thiocarbonate groups. Preferred activating groups are selected from the group consisting of formulas ((f-i) to (f-vi):

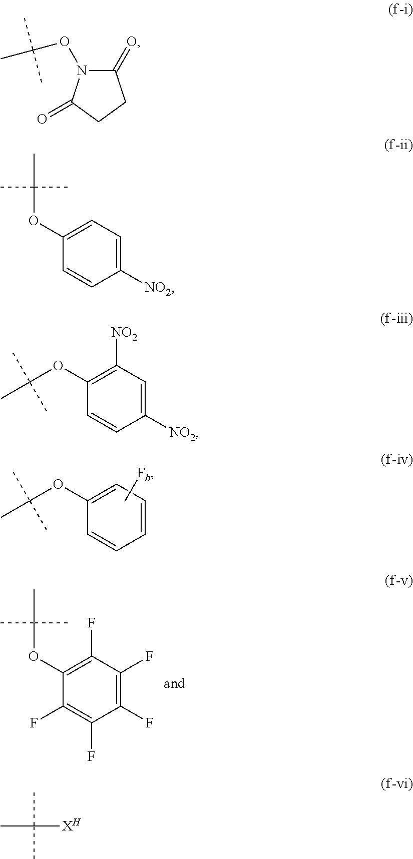

wherein
the dashed lines indicate attachment to the rest of the molecule;
b is 1, 2, 3 or 4; and
X^{H} is Cl, Br, I, or F.

Accordingly, a preferred activated ester has the formula

—(C═O)—Y¹,
wherein
Y¹ is selected from the group consisting of formulas (f-i), (f-ii), (f-iii), (f-iv), (f-v) and (f-vi).

Accordingly, a preferred activated carbamate has the formula

—N—(C═O)—Y¹,
wherein
Y¹ is selected from the group consisting of formulas (f-i), (f-ii), (f-iii), (f-iv), (f-v) and (f-vi).

Accordingly, a preferred activated carbonate has the formula

—O—(C═O)—Y¹,
wherein
Y¹ is selected from the group consisting of formulas (f-i), (f-ii), (f-iii), (f-iv), (f-v) and (f-vi).

Accordingly, a preferred activated thiocarbonate has the formula

—S—(C═O)—Y¹,
wherein
Y¹ is selected from the group consisting of formulas (f-i), (f-ii), (f-iii), (f-iv), (f-v) and (f-vi).

Accordingly, a “functional end group” is a functional group which is localized at the end of a moiety or molecule, i.e. is a terminal functional group.

If a chemical functional group is coupled to another functional group, the resulting chemical structure is referred to as “linkage”. For example, the reaction of an amine group with a carboxyl group results in an amide linkage.

As used herein, the term “protecting group” means a moiety which is reversibly connected to a functional group to render it incapable of reacting with, for example, another functional group. Suitable alcohol (—OH) protecting groups are, for example, acetyl, benzoyl, benzyl, fi-methoxyethoxymethyl ether, dimethoxytrityl, methoxymethyl ether, methoxytrityl, p-methoxybenzyl ether, methylthiomethyl ether, pivaloyl, tetrahydropyranyl, trityl, trimethylsilyl, tert-butyldimethylsilyl, tri-iso-propylsilyloxymethyl, triisopropylsilyl ether, methyl ether, and ethoxyethyl ether. Suitable amine protecting groups are, for example, carbobenzyloxy, p-methoxybenzyl carbonyl, tert-butyloxycarbonyl, 9-fluorenylmethyloxyarbonyl, acetyl, benzoyl, benzyl, carbamate, p-methoxybenzyl, 3,4-dimethoxybenzyl, p-methoxyphenyl, and tosyl. Suitable carbonyl protecting groups are, for example, acetals and ketals, acylals and dithianes. Suitable carboxylic acid protecting groups are, for example, methyl esters, benzyl esters, tert-butyl esters, 2,6-dimethylphenol, 2,6-diisopropylphenol, 2,6.-di-tert-butylphenol, silyl esters, orthoesters, and oxazoline. Suitable phosphate protecting groups are, for example, 2-cyanoethyl and methyl.

As used herein, the terms “work-up” and “working-up” refer to the series of manipulations useful and/or required to isolate and purify the product(s) of a chemical reaction, in particular of a polymerization.

As used herein, the term “polymer” means a molecule comprising repeating structural units, i.e. monomers, connected by chemical bonds in a linear, circular, branched, crosslinked or dendrimeric way or a combination thereof, which may be of synthetic or biological origin or a combination of both. It is understood that a polymer may for example also comprise functional groups. Preferably, a polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. At most, a polymer has preferably a molecular weight of 1 million Da.

As used herein, the term “polymeric” means a reagent or a moiety comprising one or more polymer(s).

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers. As used herein, the term “number average molecular weight” means the ordinary arithmetic means of the molecular weights of the individual polymers.

As used herein, the term “polymerization” or “polymerizing” means the process of reacting monomer or macromonomer reagents in a chemical reaction to form polymer chains or networks, including but not limited to hydrogels.

As used herein, the term “macromonomer” means a molecule that was obtained from the polymerization of monomer reagents.

As used herein, the term “condensation polymerization” or “condensation reaction” means a chemical reaction, in which the functional groups of two reagents react to form one single molecule, i.e. the reaction product, and a low molecular weight molecule, for example water, is released.

As used herein, the term “suspension polymerization” means a heterogeneous and/or biphasic polymerization reaction, wherein the monomer reagents are dissolved in a first solvent, forming the disperse phase which is emulsified in a second solvent, forming the continuous phase. In the present invention, the monomer reagents are the at least one backbone reagent and the at least one crosslinker reagent. Both the first solvent and the monomer reagents are not soluble in the second solvent. Such emulsion is formed by stirring, shaking, exposure to ultrasound or Microsieve™ emulsification, more preferably by stirring or Microsieve™emulsification and more preferably by stirring. This emulsion is stabilized by an appropriate emulsifier. The polymerization is initiated by addition of a base as initiator which is soluble in the first solvent. A suitable commonly known base suitable as initiator may be a tertiary base, such as tetramethylethylenediamine (TMEDA).

As used herein, the term “inert” refers to a moiety which is not chemically reactive, i.e. it does not react with other moieties or reagents. The person skilled in the art understands that the term “inert” does not per se exclude the presence of functional groups, but understands that the functional groups potentially present in an inert moiety are not reactive with functional groups of moieties/reagents brought in contact with the inert moiety in, for example, subsequent reactions. In particular, the inert moiety Z does not react with A^{x0} or A^{x2} or with functional groups present, for example, in reversible prodrug linker reagents, drugs, reversible prodrug linker moiety-biologically active moiety conjugate reagents or spacer reagents which may be covalently conjugated to the hydrogel of the present invention to obtain the hydrogel-linked prodrug of the present invention.

As used herein, the term “immiscible” means the property where two substances are not capable of combining to form a homogeneous mixture at ambient temperature and pressure, i.e. at temperature and pressure conditions typically present in a typical laboratory environment.

As used herein, the term “polyamine” means a reagent or moiety comprising more than one amine (—NH— and/or —NH₂), e.g. from 2 to 64 amines, from 4 to 48 amines, from 6 to 32 amines, from 8 to 24 amines, or from 10 to 16 amines. Particularly preferred polyamines comprise from 2 to 32 amines.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent and preferably all ethylene glycol units of said moiety or reagent are present in one block; the remaining weight percentage of the PEG-based moiety or reagent are other moieties especially selected from the group consisting of:
C₁₋₅₀ alkyl, C₂₋₅₀ alkenyl, C₂₋₅₀ alkynyl, C₃₋₁₀ cycloalkyl, 4- to 7-membered heterocyclyl, 8- to 11-membered heterobicyclyl, phenyl; naphthyl; indenyl; indanyl; and tetralinyl; and
linkages selected from the group of linkages consisting of

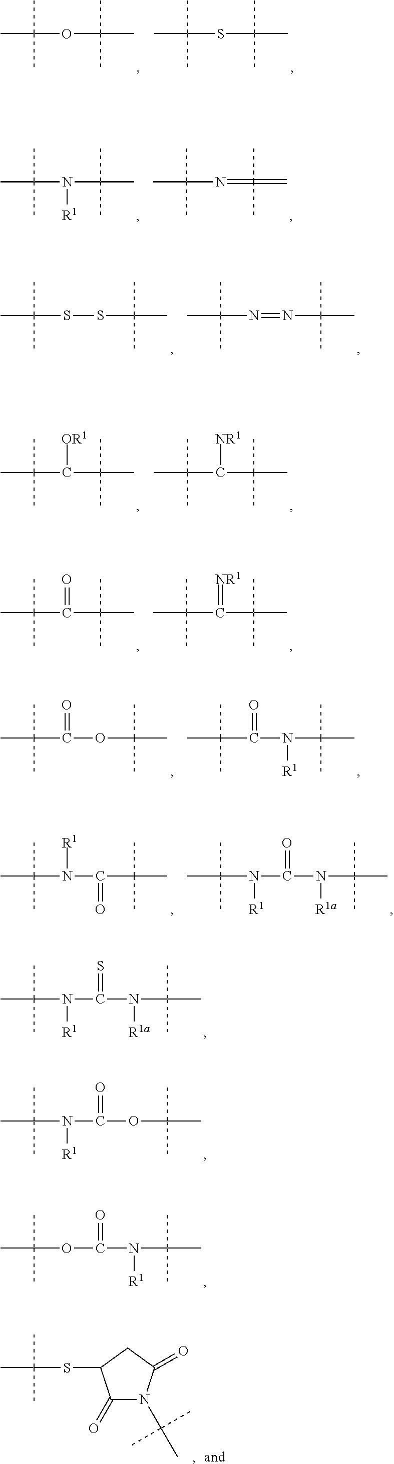

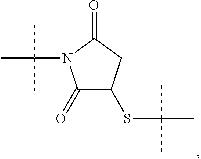

wherein
dashed lines indicate attachment to the remainder of the moiety or reagent, and
R¹ and R^{1a} are independently of each other H or C₁₋₆ alkyl.

The term “hyaluronic acid-based comprising at least X % hyaluronic acid” is used accordingly.

As used herein, the term “C₁₋₄ alkyl” alone or in combination means a straight-chain or branched alkyl group having 1 to 4 carbon atoms. If present at the end of a molecule, examples of straight-chain and branched C₁₋₄ alkyl groups are methyl, ethyl, n-propyl, isopropyl, n-butyl, isobutyl, sec-butyl and tert-butyl. When two moieties of a molecule are linked by the C₁₋₄alkyl group, then examples for such C₁₋₄alkyl groups are —CH₂—, —CH₂—CH₂—, —CH(CH₃)—, —CH₂—CH₂—CH₂—, —CH(C₂H₅)—, —C(CH₃)₂—, —CH₂—CH₂—CH₂—CH₂—, and —CH₂—CH₂—CH₂(CH₃)—. Each hydrogen atom of a C₁₋₄alkyl group may be replaced by a substituent as defined below.

As used herein, the term “C₁₋₆alkyl” alone or in combination means a straight-chain or branched alkyl group having 1 to 6 carbon atoms. If present at the end of a molecule, examples of straight-chain and branched C₁₋₆alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆alkyl group, then examples for such C₁₋₆alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆alkyl group may be replaced by a substituent as defined below.

Accordingly, as used herein, the term “C₁₋₂₀ alkyl” alone or in combination means a straight-chain or branched alkyl group having 1 to 20 carbon atoms. The term “C₈₋₁₈ alkyl” alone or in combination means a straight-chain or branched alkyl group having 8 to 18 carbon atoms. Accordingly, as used herein, the term “C₁₋₅₀ alkyl” alone or in combination means a straight-chain or branched alkyl group having 1 to 50 carbon atoms. Each hydrogen atom of a C₁₋₂₀ alkyl group, a C₈₋₁₈ alkyl group and C₁₋₅₀ alkyl group may be replaced by a substituent. In each case the alkyl group may be present at the end of a molecule or two moieties of a molecule may be linked by the alkyl group.

As used herein, the term “C₂₋₆alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆alkenyl group, then an example for such C₂₋₆alkenyl is —CH═CH—. Each hydrogen atom of a C₂₋₆alkenyl group may be replaced by a substituent as defined below. Optionally, one or more triple bond(s) may occur.

Accordingly, as used herein, the term “C₂₋₂₀ alkenyl” alone or in combination means a straight-chain or branched hydrocarbon residue comprising at least one carbon-carbon double bond having 2 to 20 carbon atoms. The term “C₂₋₅₀ alkenyl” alone or in combination means a straight-chain or branched hydrocarbon residue comprising at least one carbon-carbon double bond having 2 to 50 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the alkenyl group, then an example is e.g. —CH═CH—. Each hydrogen atom of a C₂₋₂₀ alkenyl or C₂₋₅₀ alkenyl group may be replaced by a substituent as defined below. Optionally, one or more triple bond(s) may occur.

As used herein, the term “C₂₋₆alkynyl” alone or in combination means straight-chain or branched hydrocarbon residue comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is: —C≡C—. Each hydrogen atom of a C₂₋₆alkynyl group may be replaced by a substituent as defined below. Optionally, one or more double bond(s) may occur.

Accordingly, as used herein, the term “C₂₋₂₀ alkynyl” alone or in combination means a straight-chain or branched hydrocarbon residue comprising at least one carbon-carbon triple bond having 2 to 20 carbon atoms and “C₂₋₅₀ alkynyl” alone or in combination means a straight-chain or branched hydrocarbon residue comprising at least one carbon-carbon triple bond having 2 to 50 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₂₀ alkynyl or C₂₋₅₀ alkynyl group may be replaced by a substituent as defined below. Optionally, one or more double bond(s) may occur.

As used herein, the terms “C₃₋₈cycloalkyl” or “C₃₋₈cycloalkyl ring” means a cyclic alkyl chain having 3 to 8 carbon atoms, which may be saturated or unsaturated, e.g. cyclopropyl, cyclobutyl, cyclopentyl, cyclohexyl, cyclohexenyl, cycloheptyl, cyclooctyl. Each hydrogen atom of a cycloalkyl carbon may be replaced by a substituent as defined below. The term “C₃₋₈cycloalkyl” or “C₃₋₈cycloalkyl ring” also includes bridged bicycles like norbonane or norbonene. Accordingly, “C₃₋₅cycloalkyl” means a cycloalkyl having 3 to 5 carbon atoms and C₃₋₁₀ cycloalkyl having 3 to 10 carbon atoms.

Accordingly, as used herein, the term “C₃₋₁₀ cycloalkyl” means a carbocyclic ring system having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl, cyclodecyl. The term “C₃₋₁₀ cycloalkyl” also includes at least partially saturated carbomono- and -bicycles.

As used herein, the term “halogen” means fluoro, chloro, bromo or iodo. Particularly preferred is fluoro or chloro.

As used herein, the term “4- to 7-membered heterocyclyl” or “4- to 7-membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 4- to 7-membered heterocycles include but are not limited to azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 4- to 7-membered heterocyclyl or 4- to 7-membered heterocyclic group may be replaced by a substituent as defined below.

As used herein, the term “8- to 11-membered heterobicyclyl” or “8- to 11-membered heterobicycle” means a heterocyclic system of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquino line, decahydroquino line, isoquinoline, decahydroisoquino line, tetrahydroisoquinoline, dihydroisoquinoline, benzazepine, purine and pteridine. The term 8-to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

The term “substituted” means that one or more —H atom(s) of a molecule or moiety are replaced by a different atom or a group of atoms, which are referred to as “substituent”. Suitable substituents are halogen; CN; COOR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); SR⁹; N(R⁹R^{9a}); NO₂; OC(O)R⁹; N(R⁹)C(O)R^{9a}; N(R⁹)S(O)₂R^{9a}; N(R⁹)S(O)R^{9a}; N(R⁹)C(O)OR^{9a}; N(R⁹)C(O)N(R^{9a}R^{9b}); OC(O)N(R⁹R^{9a}); T; C₁₋₅₀ alkyl; C₂₋₅₀ alkenyl; or C₂₋₅₀ alkynyl, which T; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more group(s) selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});
wherein
R⁹, R^{9a}, R^{9b} are independently selected from the group consisting of H; T; C₁₋₅₀ alkyl; C₂₋₅₀ alkenyl; and C₂₋₅₀ alkynyl, which T; C₁₋₅₀ alkyl; C₂₋₅₀ alkenyl; and C₂₋₅₀ alkynyl are optionally substituted with one or more R¹⁰, which are the same or different and which C₁₋₅₀ alkyl; C₂₋₅₀ alkenyl; and C₂₋₅₀ alkynyl are optionally interrupted by one or more group(s) selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{1a});
T is selected from the group consisting of phenyl; naphthyl; indenyl; indanyl; tetralinyl; C₃₋₁₀ cycloalkyl; 4- to 7-membered heterocyclyl; and 8- to 11-membered heterobicyclyl, wherein T is optionally substituted with one or more R¹⁰, which are the same or different;
R¹⁰ is halogen; CN; oxo (═O); COOR¹²; OR¹²; C(O)R¹²; C(O)N(R¹²R^{12a}); S(O)₂N(R¹²R^{12a}); S(O)N(R¹²R^{12a}); S(O)₂R¹²; S(O)R¹²; N(R¹²)S(O)₂N(R^{12a}R^{12b}); SR¹²; N(R¹²R^{12a}); NO₂; OC(O)R¹²; N(R¹²)C(O)R^{12a}; N(R¹²)S(O)₂R^{12a}; N(R¹²)S(O)R^{12a}; N(R¹²)C(O)OR^{12a}; N(R¹²)C(O)N(R^{12a}R^{12b}); OC(O)N(R¹²R^{12a}); or C₁₋₆alkyl, which C₁₋₆alkyl is optionally substituted with one or more halogen, which are the same or different;
R¹¹, R^{11a}, R¹², R^{12a}, R^{12b} are independently of each other selected from the group consisting of H; and C₁₋₆alkyl, which C₁₋₆alkyl is optionally substituted with one or more halogen, which are the same or different.

In one embodiment R⁹, R^{9a}, R^{9b} are independently of each other H.

In one embodiment R¹⁰ is C₁₋₆alkyl.

In one embodiment T is phenyl.

Preferably, a maximum of 6 —H atoms of a moiety or molecule are independently replaced by a substituent, e.g. 5 —H atoms are independently replaced by a substiuent, 4 —H atoms are independently replaced by a substituent, 3 —H atoms are independently replaced by a substituent, 2 —H atoms are independently replaced by a substituent, or 1 —H atom is replaced by a substituent.

As used herein, the term “interrupted” means that between two carbon atoms or at the end of a carbon chain between the respective carbon atom and the hydrogen atom one or more atom(s) are inserted.

As used herein, the term “prodrug” means a compound that undergoes biotransformation before exhibiting its pharmacological effects. Prodrugs can thus be viewed as biologically active moieties connected to specialized non-toxic protective groups used in a transient manner to alter or to eliminate undesirable properties in the parent molecule. This also includes the enhancement of desirable properties in the drug and the suppression of undesirable properties.

As used herein, the term “carrier-linked prodrug” means a prodrug that contains a reversible linkage of a biologically active moiety with a carrier group and which carrier improves the physicochemical or pharmacokinetic properties of the biologically active moiety and which carrier is removed in vivo, usually by a hydrolytic cleavage. Preferably, the carrier is a polymer.

Accordingly, the term “hydrogel-linked prodrug” refers to a carrier-linked prodrug in which the carrier is a hydrogel.

In order for a hydrogel to be “suitable as a carrier in a hydrogel-linked prodrug” such hydrogel needs functional groups for conjugating reversible prodrug linker reagents or reversible prodrug linker moiety-biologically active moiety conjugate reagents to said hydrogel.

As used herein, the term “reversible prodrug linker” means a moiety which on its one end is attached to a backbone moiety of the hydrogel either directly or through a spacer moiety and on another end is attached to a biologically active moiety through a reversible linkage.

A “biodegradable linkage” or “reversible linkage” is is a linkage that is enzymatically and/or non-enzymatically hydrolytically degradable, i.e. cleavable, under physiological conditions (aqueous buffer at pH 7.4, 37° C.) with a half-life ranging from one hour to twelve months. Preferably, a biodegradable linkage is non-enzymatically hydrolytically degradable, i.e. degradable independent of enzymatic activity, under physiological conditions with a half-life ranging from one hour to twelve months.

In contrast, a “permanent linkage” is non-enzymatically hydrolytically degradable under physiological conditions (aqueous buffer at pH 7.4, 37° C.) with a half-life of more than twelve months.

As used herein, the term “pharmaceutical composition” means one or more active ingredients, i.e. drugs or prodrugs, and one or more inert ingredients, the so-called excipients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the hydrogel-linked prodrug releasing tag moiety-biologically active moiety conjugates of the present invention and one or more pharmaceutically acceptable excipient(s).

As used herein, the term “excipient” refers to a diluent, adjuvant, or vehicle with which the active ingredient is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-l-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. An oral formulation can include standard excipients such as pharmaceutical grades of mannitol, lactose, starch, magnesium stearate, sodium saccharine, cellulose, magnesium carbonate, etc. Examples of suitable pharmaceutical excipients are described in “Remington's Pharmaceutical Sciences” by E.W. Martin. Such compositions will contain a therapeutically effective amount of the drug or prodrug, together with a suitable amount of excipient, so as to provide the form for proper administration to the patient. The formulation should suit the mode of administration.

In general the term “comprise” or “comprising” also encompasses “consist of” or “consisting of”.

Step (a)

The hydrogel of step (a) may be any hydrogel known in the art that is suitable as a carrier for hydrogel-linked prodrugs. It is understood that the functional groups A^{x0} and A^{x2} are used to covalently conjugate reversible prodrug linker moieties or reversible prodrug linker moiety-biologically active moiety conjugate reagents to the hydrogel.

Preferably, A^{x0} is selected from the group consisting of maleimide, amine (—NH₂ or —NH—), hydroxyl (—OH), thiol, carboxyl (—COOH) and activated carboxyl (—COY¹, wherein Y¹ is selected from formulas (f-i) to (f-vi):

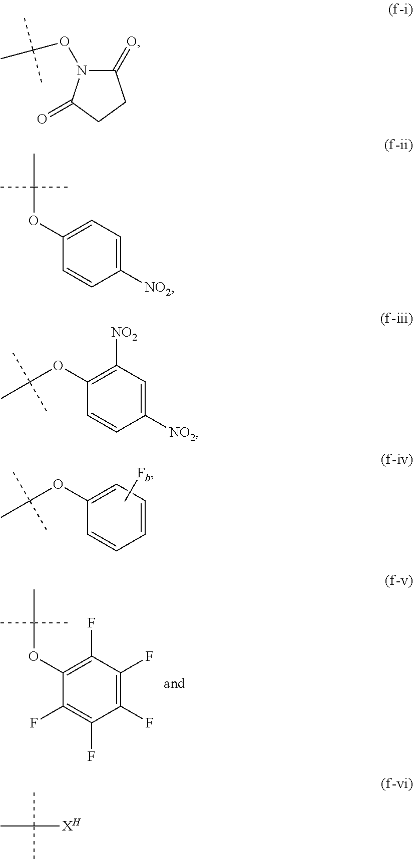

wherein
the dashed lines indicate attachment to the rest of the molecule,
b is 1, 2, 3 or 4;
X^{H} is Cl, Br, I, or F).

More preferably, A^{x0} is selected from the group consisting of maleimide, amine (—NH₂ or —NH—), hydroxyl (—OH), carboxyl (—COOH) and activated carboxyl (—COY¹, wherein Y¹ is selected from formulas (f-i) to (f-vi):

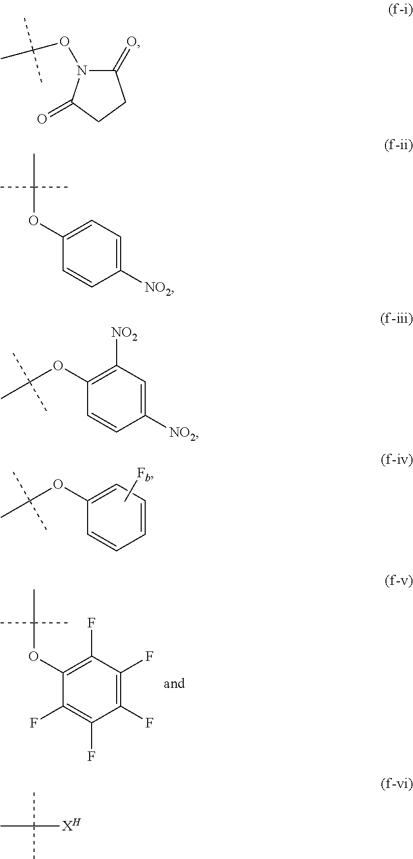

wherein
the dashed lines indicate attachment to the rest of the molecule,
b is 1, 2, 3 or 4;
X^{H} is Cl, Br, I, or F).

More preferably, A^{x0} is an amine or maleimide.

It is equally preferred that A^{x0} is thiol.

Preferably, the hydrogel of step (a) is a shaped article, such as a coating, mesh, stent, nanoparticle or a microparticle. More preferably, the hydrogel is in the form of microparticular beads having a diameter from 1 to 1000 micrometer, more preferably with a diameter from 10 to 300 micrometer, even more preferably with a diameter from 20 and 150 micrometer and most preferably with a diameter from 30 to 130 micrometer. The afore-mentioned diameters are measured when the hydrogel microparticles are fully hydrated in water.

In one embodiment the hydrogel of step (a) is a hydrogel as disclosed in W02006003014A2 which is incorporated by reference herein.

In another embodiment the hydrogel of step (a) is a hydrogel as disclosed in W02011012715A1 which is incorporated by reference herein.

In a preferred embodiment the hydrogel of step (a) is obtainable by a process comprising the steps of:
(a-i) providing a mixture comprising
   (a-ia) at least one backbone reagent, wherein the at least one backbone reagent has a molecular weight ranging from 1 to 100 kDa, and comprises at least three functional groups A^{x0}, wherein each A^{x0} is a maleimide, amine (—NH₂ or —NH—), hydroxyl (—OH), carboxyl (—COOH) or activated carboxyl (—COY¹, wherein Y¹ is selected from formulas (f-i) to (f-vi):

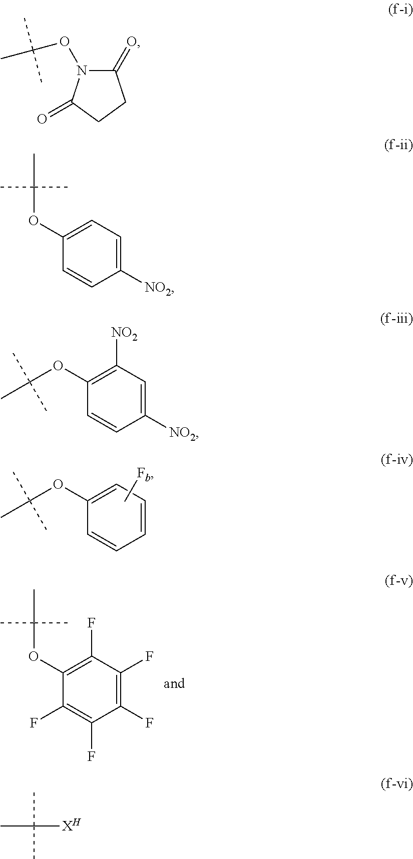

wherein
the dashed lines indicate attachment to the rest of the molecule,
b is 1, 2, 3 or 4,
X^{H} is Cl, Br, I, or F);
(a-ib) at least one crosslinker reagent, wherein the at least one crosslinker reagent has a molecular weight ranging from 0.2 to 40 kDa and comprises at least two functional end groups selected from the group consisting of activated ester groups, activated carbamate groups, activated carbonate groups, activated thiocarbonate groups, amine groups and thiol groups;
in a weight ratio of the at least one backbone reagent to the at least one crosslinker reagent ranging from 1:99 to 99:1 and wherein the molar ratio of A^{x0} to functional end groups is >1;
(a-ii) polymerizing the mixture of step (a-i) to a hydrogel; and
(a-iii) optionally working-up the hydrogel of step (a-ii).

Equally preferably, A^{x0} of step (a-ia) is thiol.

The Backbone Reagent of Step (a-ia)

The at least one backbone reagent has a molecular weight ranging from 1 to 100 kDa, preferably from 2 to 50 kDa, more preferably from 5 and 30 kDa, even more preferably from 5 to 25 kDa and most preferably from 5 to 15 kDa.

In one embodiment the at least one backbone reagent of step (a-ia) is present in the form of its acidic salt, preferably in the form of an acid addition salt, if A^{x0} is amine. Suitable acid addition salts are formed from acids which form non-toxic salts. Examples include but are not limited to acetate, aspartate, benzoate, besylate, bicarbonate, carbonate, bisulphate, sulphate, borate, camsylate, citrate, edisylate, esylate, formate, fumarate, gluceptate, gluconate, glucuronate, hexafluorophosphate, hibenzate, hydrochloride, hydrobromide, hydroiodide, isethionate, lactate, malate, maleate, malonate, mesylate, methylsulphate, naphthylate, nicotinate, nitrate, orotate, oxalate, palmitate, pamoate, phosphate, hydrogen phosphate, dihydrogen phosphate, sacharate, stearate, succinate, tartrate and tosylate. Particularly preferred, the backbone reagent is present in the form of its hydrochloride salt. The at least one backbone reagent of step (a-ia) comprises one or more polymer(s) selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

Preferably, the at least one backbone reagent of step (a-ia) is PEG-based comprising at least 10% PEG or is hyaluronic acid-based comprising at least 20% hyaluronic acid.

In a preferred embodiment, the at least one backbone reagent of step (a-ia) is hyaluronic acid-based comprising at least 20% hyaluronic acid, more preferably, comprising at least 40% hyaluronic acid, even more preferably, at least 60% hyaluronic acid, even more preferred at least 80% hyaluronic acid.

Preferably, in such hyaluronic acid-comprising backbone reagent of step (a-ia) each A^{x0} is an amine.

In another preferred embodiment, the at least one backbone reagent of step (a-ia) is PEG-based comprising at least 10% PEG, preferably at least 20% PEG, even more preferably at least 30%, even more preferably at least 40% PEG, even more preferably at least 50% PEG, and most preferably at least 60%.

Preferably, in such PEG-based backbone reagent of step (a-ia) each A^{x0} is an amine or maleimide and most preferably each A^{x0} is an amine.

In one embodiment, the at least one backbone reagent of step (a-ia) is selected from the group consisting of
(i) a compound of formula (IIIa)

B(-(A⁰)ₓ₁-(SP¹)ₓ₂-A¹-P-A²-Hyp¹)ₓ (IIIa),
wherein
B is a branching core,
SP¹ is a spacer moiety selected from the group consisting of C₁₋₆ alkyl, C₂₋₆ alkenyl and C₂₋₆alkynyl,
P is a PEG-based polymeric chain comprising at least 80% PEG, preferably at least 85% PEG, more preferably at least 90% PEG and most preferably at least 95% PEG,
Hyp¹ is a moiety comprising an amine (—NH₂ and/or —NH—) or a polyamine comprising at least two amines (—NH₂ and/or —NH—),
x is an integer from 3 to 16,
x1, x2 are independently of each other 0 or 1, provided that x1 is 0, if x2 is 0,
A⁰, A¹, A² are independently of each other selected from the group consisting of

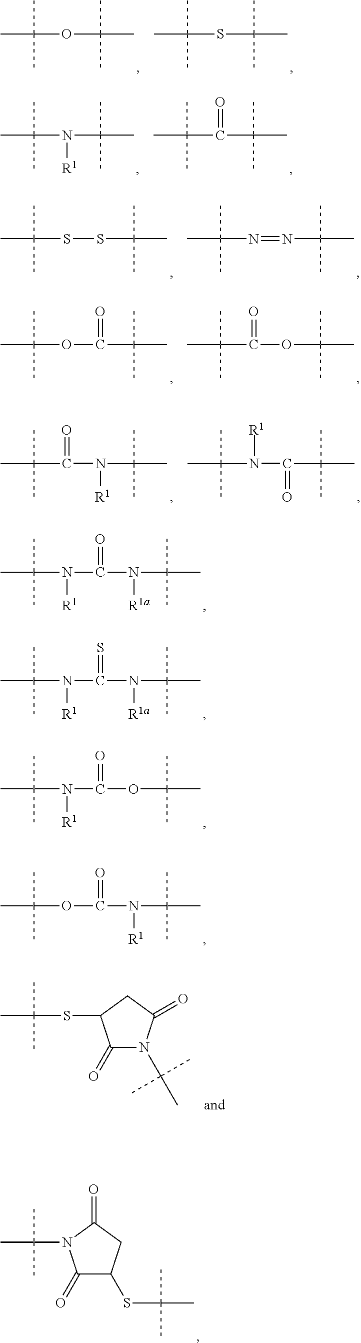

wherein R¹ and R^{1a} are independently of each other H or C₁₋₆alkyl;
(ii) a compound of formula (IIIb)

Hyp²-A³-P-A⁴-Hyp³ (IIIb),
wherein
P is defined as above in the compound of formula (IIIa),
Hyp², Hyp³ are independently of each other a polyamine comprising at least two amines (—NH₂ and/or —NH—), and
A³ and A⁴ are independently selected from the group consisting of

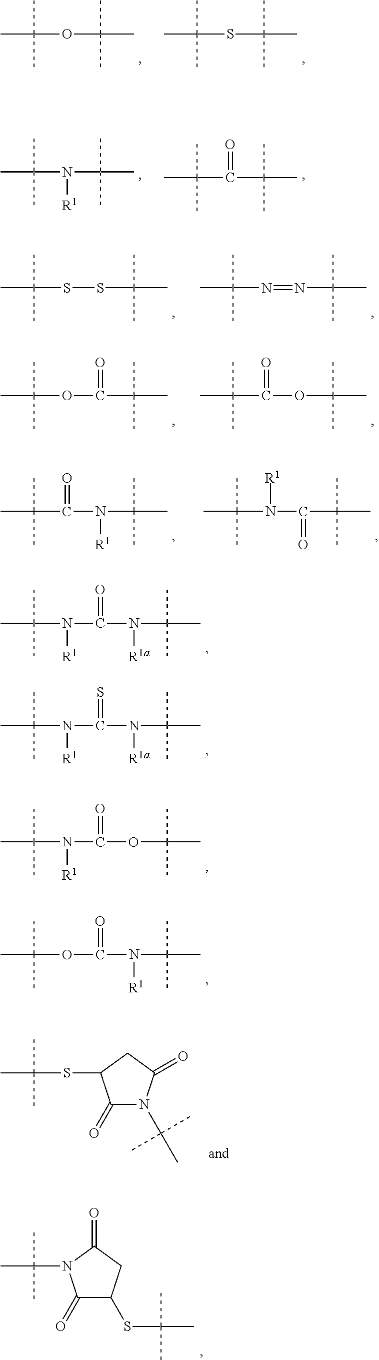

wherein R¹ and R^{1a} are independently of each other H or C₁₋₆alkyl;
(iii) a compound of formula (IIIc)

P¹-A⁵-Hyp⁴ (IIIc),
wherein
P¹ is a PEG-based polymeric chain comprising at least 80% PEG, preferably at least 85% PEG, more preferably at least 90% PEG and most preferably at least 95% PEG,
Hyp⁴ is a polyamine comprising at least three amines (—NH₂ and/or —NH), and
A⁵ is selected from the group consisting of

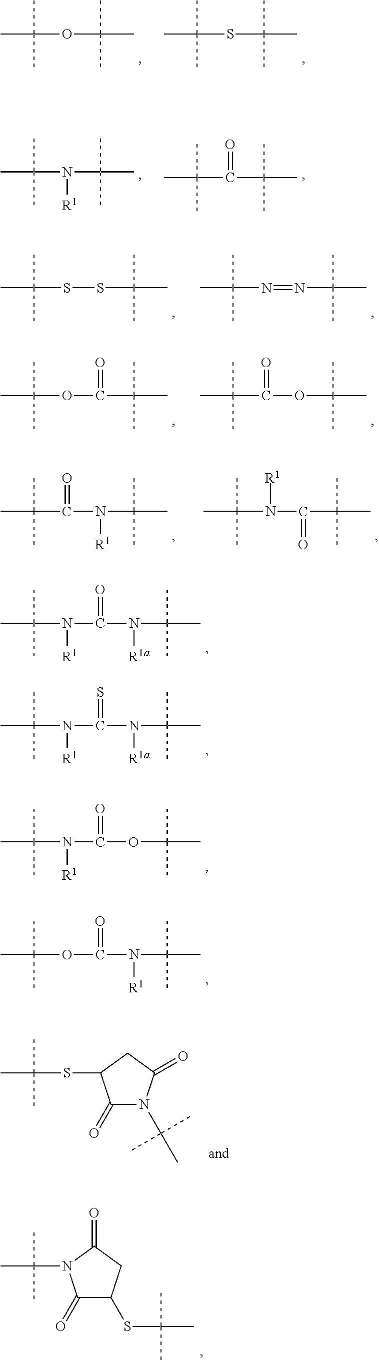

wherein R¹ and R^{1a} are independently of each other H or C₁₋₆ alkyl;
and
(iv) a compound of formula (IIId)

T¹-A⁶-Hyp⁵ (IIId),
wherein
Hyp⁵ is a polyamine comprising at least three amines (—NH₂ and/or —NH), and
A⁶ is selected from the group consisting of

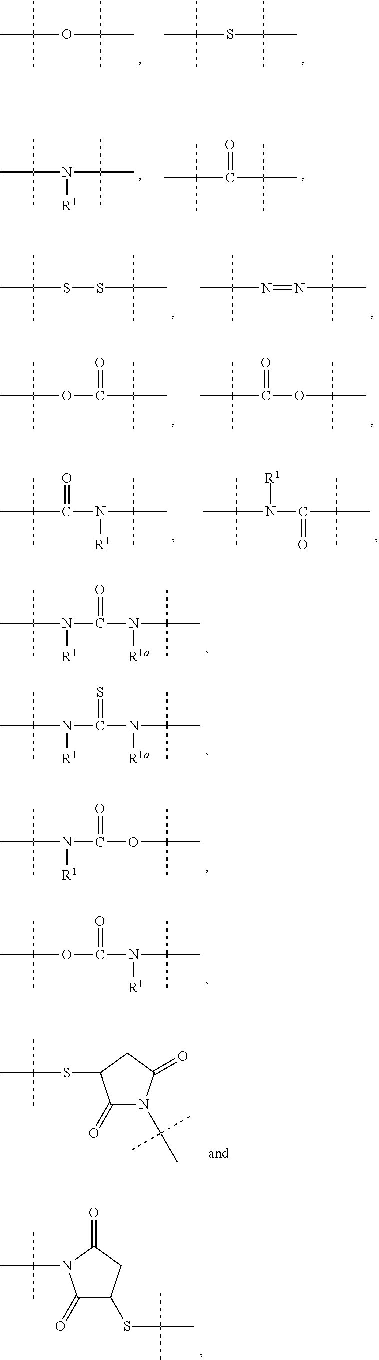

wherein R¹ and R^{1a} are independently of each other H or C₁₋₆alkyl; and
T¹ is selected from the group consisting of C₁₋₅₀ alkyl, C₂₋₅₀ alkenyl and C₂₋₅₀ alkynyl, which C₁₋₅₀ alkyl, C₂₋₅₀ alkenyl or C₂₋₅₀ alkynyl are optionally interrupted by one or more group(s) selected from the group consisting of —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl.

In the following sections the term “Hyp^{x}” refers to Hyp¹, Hyp², Hyp³, Hyp⁴ and Hyp⁵ collectively.

Preferably, the backbone reagent is a compound of formula (IIIa), (IIIb) or (IIIc), more preferably the backbone reagent is a compound of formula (IIIa) or (IIIc), and most preferably the backbone reagent is a compound of formula (IIIa).

In a preferred embodiment, the backbone reagent is of formula (IIIa) and x is 4, 6 or 8, more preferably x is 4 or 8 and most preferably x is 4.

In a preferred embodiment A⁰, A¹, A², A³, A⁴, A⁵ and A⁶ of formulas (IIIa) to (IIId) are selected from the group consisting of

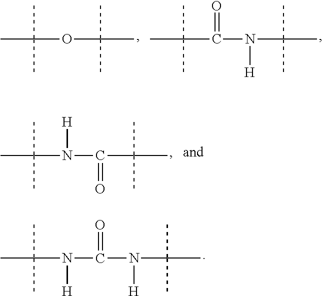

Preferably, A⁰ of formula (IIIa) is selected from the group consisting of

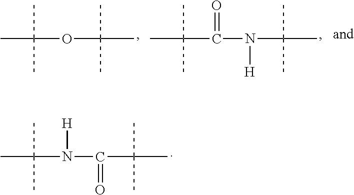

Preferably, A¹ of formula (IIIa) is selected from the group consisting of

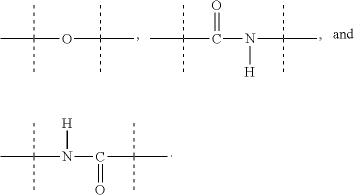

Preferably, A² of formula (IIIa) is selected from the group consisting of

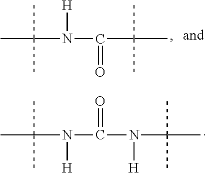

Preferably, A³ of formula (IIIb) is selected from the group consisting of

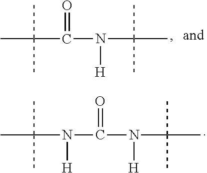

Preferably, A⁴ of formula (IIIb) is selected from the group consisting of

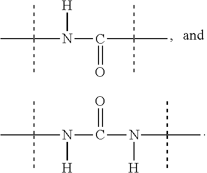

Preferably, A⁵ of formula (IIIc) is selected from the group consisting of

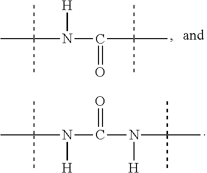

Preferably, A⁶ of formula (IIId) is selected from the group consisting of

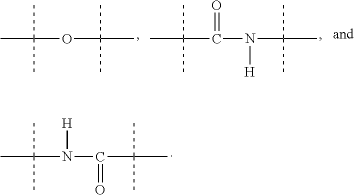

Preferably, in a compound of formula (IIId), T¹ is H or C₁₋₆alkyl.

SP¹ is a spacer moiety selected from the group consisting of C₁₋₆ alkyl, C₂₋₆alkenyl and C₂₋₆ alkynyl. Preferably SP¹ is —CH₂—, —CH₂—CH₂—, —CH(CH₃)—, —CH₂—CH₂—CH₂—, —CH(C₂H₅)—, —C(CH₃)₂—, —CH═CH— or —CH═CH—, and most preferably SP¹ is —CH₂—, —CH₂—CH₂— or —CH═CH—.

In one embodiment B of formula (IIIa) is selected from the group consisting of:

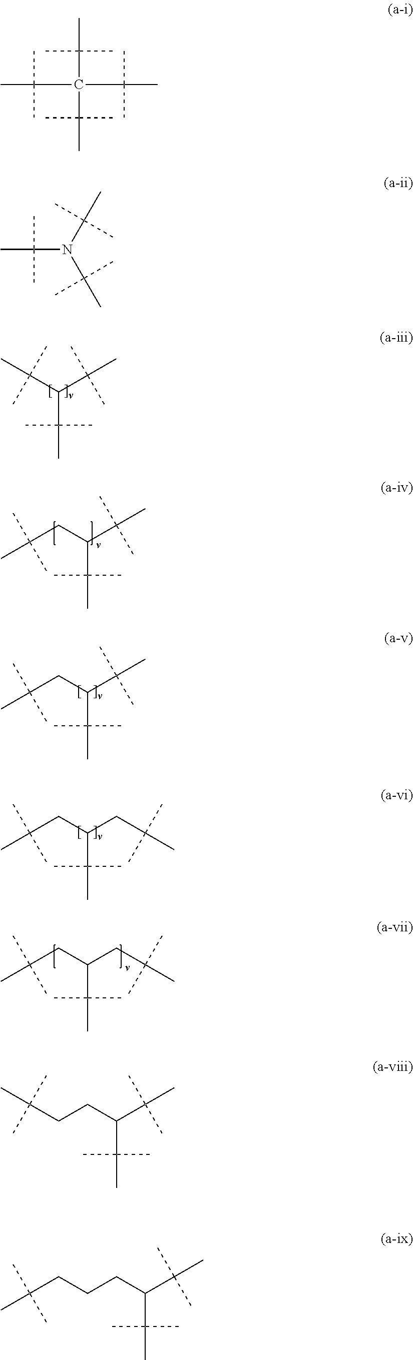

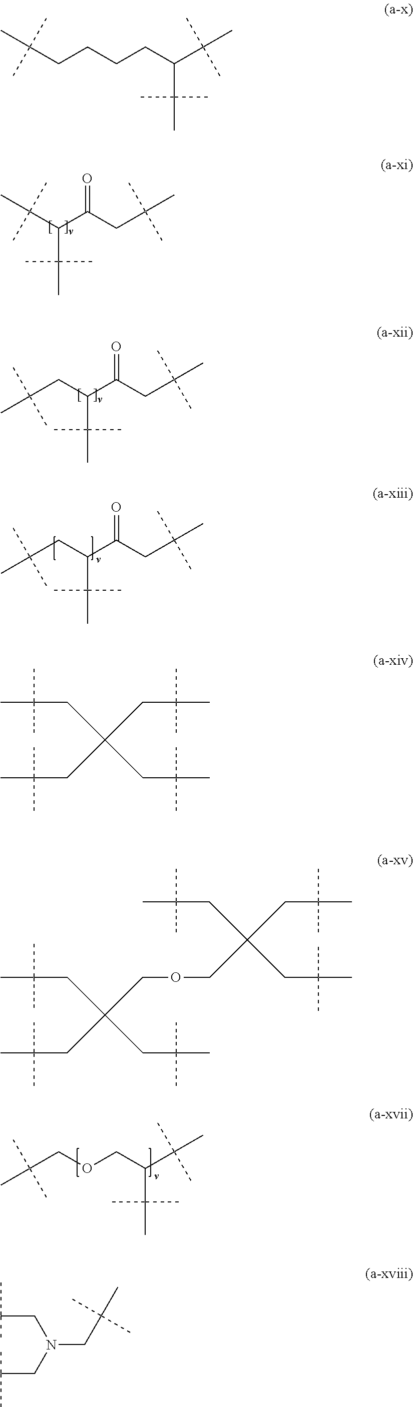

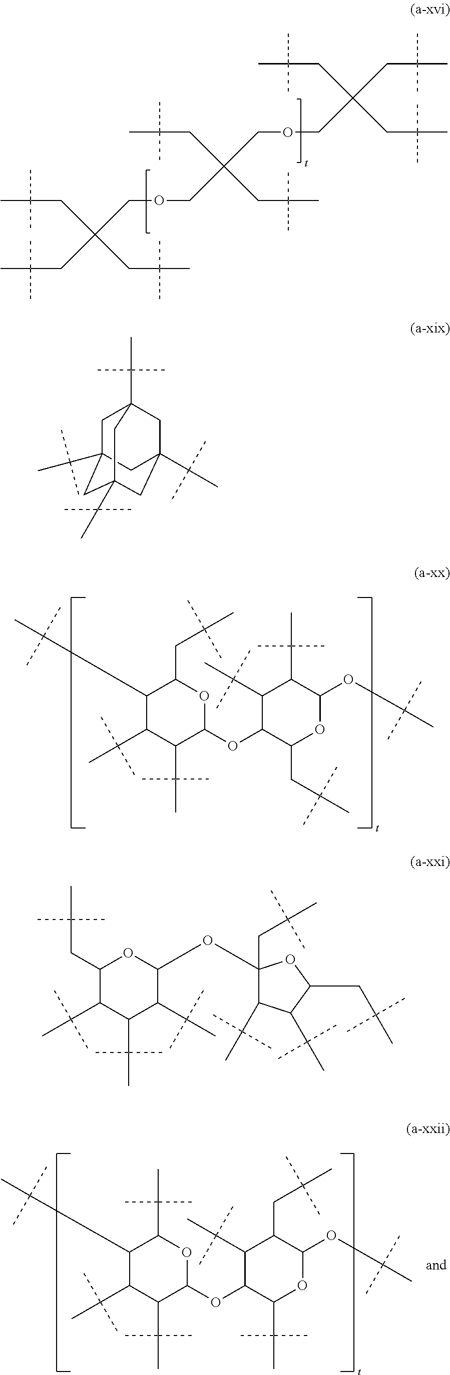

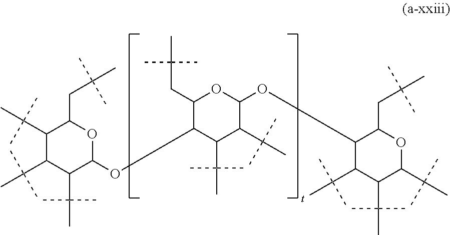

wherein
dashed lines indicate attachment to A⁰ or, if x1 and x2 are both 0, to A¹,
t is 1 or 2; preferably t is 1,
v is 1, 2, 3, 4, 5, 6, 7, 8, 9, 10, 11, 12, 13 or 14; preferably, v is 2, 3, 4, 5, 6; more preferably, v is 2, 4 or 6; most preferably, v is 2.

In a preferred embodiment, B has a structure of formula (a-i), (a-ii), (a-iii), (a-iv), (a-v), (a-vi), (a-vii), (a-viii), (a-ix), (a-x), (a-xiv), (a-xv) or (a-xvi). More preferably, B has a structure of formula (a-iii), (a-iv), (a-v), (a-vi), (a-vii), (a-viii), (a-ix), (a-x) or (a-iv). Most preferably, B has a structure of formula (a-xiv).

A preferred combination of B and A⁰, or, if x1 and x2 are both 0, of B and A¹, is selected from the group consisting of the following structures:

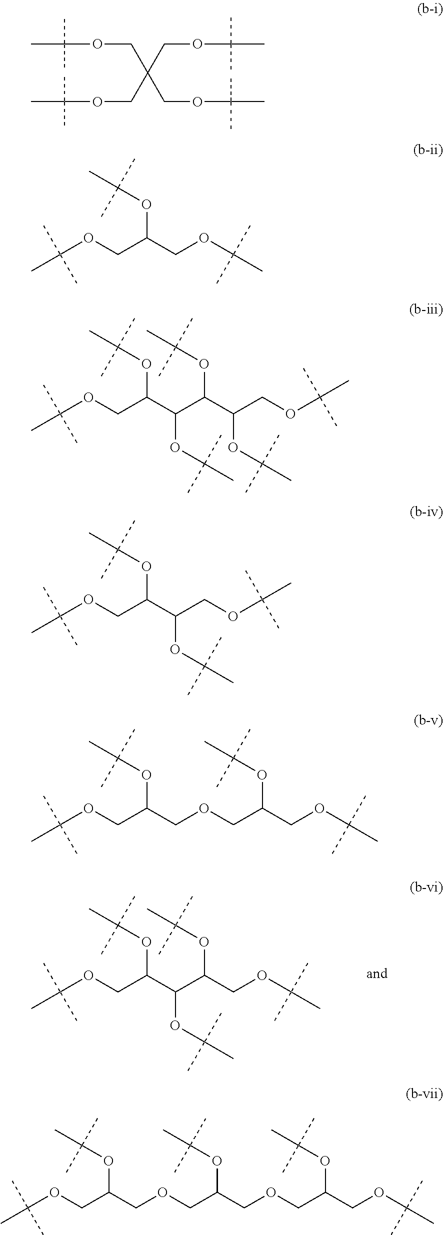

wherein
dashed lines indicate attachment to SP¹ or, if x1 and x2 are both 0, to P.

More preferably, the combination of B and A⁰ or, if x1 and x2 are both 0, the combination of B and A¹, is of formula (b-i), (b-iv), (b-vi) or (b-viii) and most preferably is of formula (b-i).

In one embodiment, x1 and x2 of formula (IIIa) are both 0.

In one embodiment, the PEG-based polymeric chain P has a molecular weight from 0.3 kDa to 40 kDa; e.g. from 0.4 to 35 kDa, from 0.6 to 38 kDa, from 0.8 to 30 kDa, from 1 to 25 kDa, from 1 to 15 kDa or from 1 to 10 kDa. Most preferably P has a molecular weight from 1 to 10 kDa.

In one embodiment, the PEG-based polymeric chain P¹ has a molecular weight from 0.3 kDa to 40 kDa; e.g. from 0.4 to 35 kDa, from 0.6 to 38 kDA, from 0.8 to 30 kDa, from 1 to 25 kDa, from 1 to 15 kDa or from 1 to 10 kDa. Most preferably P¹ has a molecular weight from 1 to 10 kDa.

In one embodiment, P of formula (IIIa) or (IIIb) is of formula (c-i):

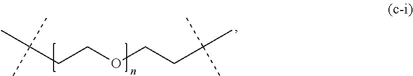

wherein n ranges from 6 to 900, more preferably n ranges from 20 to 700 and most preferably n ranges from 20 to 250.

In one embodiment, P¹ of formula (IIIc) has the structure of formula (c-ii):

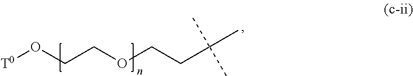

wherein
n ranges from 6 to 900, more preferably n ranges from 20 to 700 and most preferably n ranges from 20 to 250;
T⁰ is selected from the group consisting of C₁₋₆alkyl, C₂₋₆alkenyl and C₂₋₆ alkynyl, which C₁₋₆alkyl, C₂₋₆alkenyl and C₂₋₆alkynyl are optionally interrupted by one or more group(s) selected from the group consisting of —NH—, —N(C₁₋₄alkyl)-, —O—, —S—, —C(O)—, —C(O)NH—, —C(O)N(C₁₋₄alkyl)-, —O—C(O)—, —S(O)— and —S(O)₂—.

In one embodiment, the moiety Hyp^{x} of formulas (IIIa) to (IIId) is a polyamine and preferably comprises in bound form and, where applicable, in R- and/or S-configuration, a moiety of formulas (d-i), (d-ii), (d-iii) and/or (d-iv):

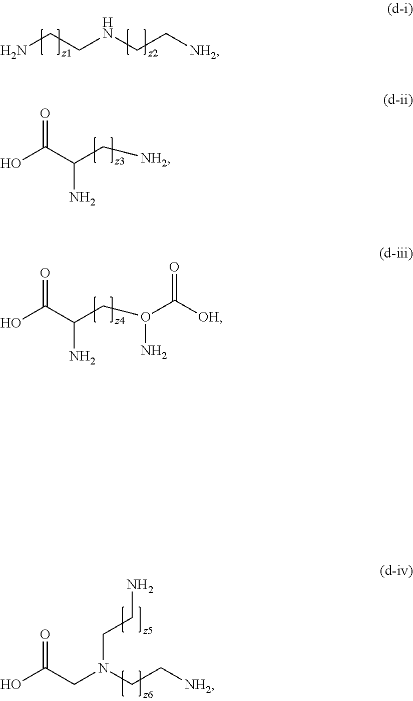

wherein
z1, z2, z3, z4, z5, z6 are independently of each other 1, 2, 3, 4, 5, 6, 7 or 8.

More preferably, Hyp^{x} comprises in bound form and in R- and/or S-configuration lysine, ornithine, diaminoproprionic acid and/or diaminobutyric acid. Hyp^{x} comprises in bound form and in R- and/or S-configuration lysine.

Preferably, Hyp^{x} has a molecular weight from 40 Da to 30 kDa, preferably from 0.3 kDa to 25 kDa, more preferably from 0.5 kDa to 20 kDa, even more preferably from 1 kDa to 20 kDa and most preferably from 2 kDa to 15 kDa.

Hyp^{x} is preferably selected from the group consisting of:

a moiety of formula (e-i)

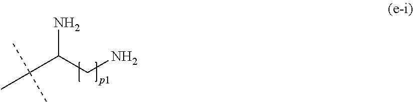

wherein
p1 is an integer from 1 to 5, preferably p1 is 4, and
the dashed line indicates attachment to A² if the backbone reagent is of formula (IIIa) and to A³ or A⁴ if the backbone reagent is of formula (IIIb);

a moiety of formula (e-ii)

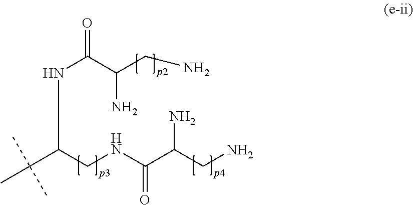

wherein
p2, p3 and p4 are identical or different and each is independently of the others an integer from 1 to 5, preferably p2, p3 and p4 are 4, and
the dashed line indicates attachment to A² if the backbone reagent is of formula (IIIa), to A³ or A⁴ if the backbone reagent is of formula (IIIb), to A⁵ if the backbone is of formula (IIIc) and to A⁶ if the backbone reagent is of formula (IIId);

a moiety of formula (e-iii)

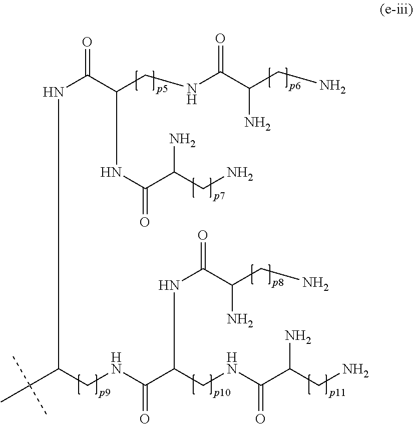

wherein
p5 to p11 are identical or different and each is independently of the others an integer from 1 to 5, preferably p5 to p11 are 4, and
the dashed line indicates attachment to A² if the backbone reagent is of formula (IIIa), to A³ or A⁴ if the backbone reagent is of formula (IIIb), to A⁵ if the backbone reagent is of formula (IIIc) and to A⁶ if the backbone reagent is of formula (IIId);

a moiety of formula (e-iv)

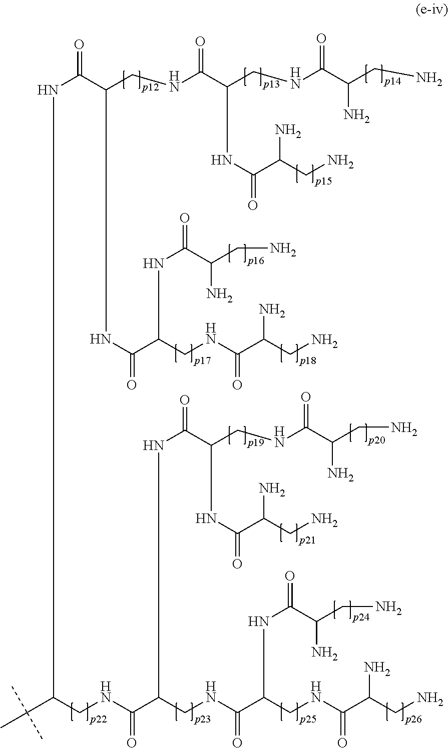

wherein
p12 to p26 are identical or different and each is independently of the others an integer from 1 to 5, preferably p12 to p26 are 4, and
the dashed line indicates attachment to A² if the backbone reagent is of formula (IIIa), to A³ or A⁴ if the backbone reagent is of formula (IIIb), to A⁵ if the backbone is of formula (IIIc) and to A⁶ if the backbone reagent is of formula (IIId);

a moiety of formula (e-v)

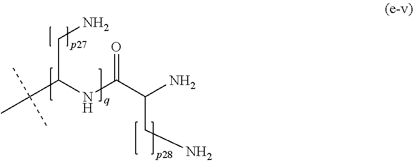

wherein
p27 and p28 are identical or different and each is independently of the other an integer from 1 to 5, preferably p27 and p28 are 4,
q is an integer from 1 to 8, preferably q is 2 or 6 and most preferably q is 6, and
the dashed line indicates attachment to A² if the backbone reagent is of formula (IIIa), to A³ or A⁴ if the backbone reagent is of formula (IIIb), to A⁵ if the backbone reagent is of formula (IIIc) and to A⁶ if the backbone reagent is of formula (IIId);

a moiety of formula (e-vi)

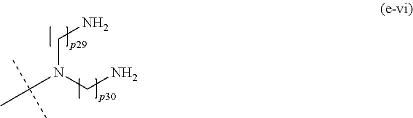

wherein
p29 and p30 are identical or different and each is independently of the other an integer from 2 to 5, preferably p29 and p30 are 3, and
the dashed line indicates attachment to A² if the backbone reagent is of formula (IIIa), to A³ or A⁴ if the backbone reagent is of formula (IIIb), to A⁵ if the backbone reagent is of formula (IIIc) and to A⁶ if the backbone reagent is of formula (IIId);

a moiety of formula (e-vii)

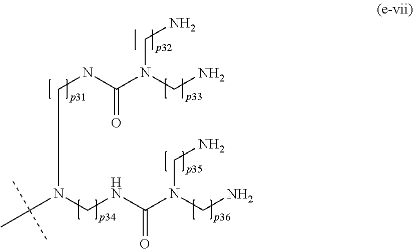

wherein
p31 to p36 are identical or different and each is independently of the others an integer from 2 to 5, preferably p31 to p36 are 3, and
the dashed line indicates attachment to A² if the backbone reagent is of formula (IIIa), to A³ or A⁴ if the backbone reagent is of formula (IIIb), to A⁵ if the backbone reagent is of formula (IIIc) and to A⁶ if the backbone reagent is of formula (IIId);

a moiety of formula (e-viii)

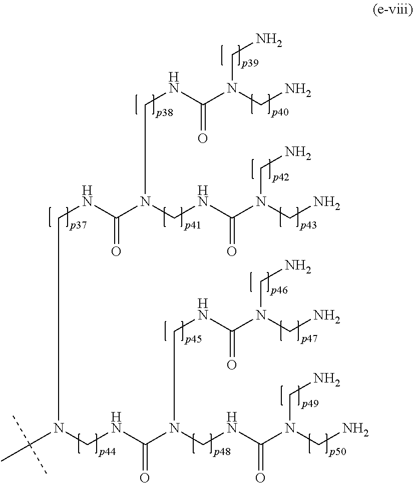

wherein
p37 to p50 are identical or different and each is independently of the others an integer from 2 to 5, preferably p37 to p50 are 3, and
the dashed line indicates attachment to A² if the backbone reagent is of formula (IIIa), to A³ or A⁴ if the backbone reagent is of formula (IIIb), to A⁵ if the backbone reagent is of formula (IIIc) and to A⁶ if the backbone reagent is of formula (IIId); and

a moiety of formula (e-ix):

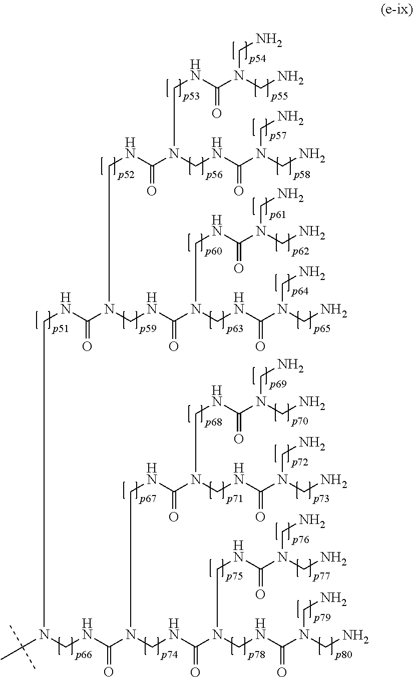

wherein
p51 to p80 are identical or different and each is independently of the others an integer from 2 to 5, preferably p51 to p80 are 3, and
the dashed line indicates attachment to A² if the backbone reagent is of formula (IIIa), to A³ or A⁴ if the backbone reagent is of formula (IIIb), to A⁵ if the backbone reagent is of formula (IIIc) and to A⁶ if the backbone reagent is of formula (IIId); and

wherein the moieties (e-i) to (e-v) may at each chiral center be in either R- or S-configuration, preferably, all chiral centers of a moiety (e-i) to (e-v) are in the same configuration.

Preferably, Hyp^{x} is of formula (e-i), (e-ii), (e-iii), (e-iv), (e-vi), (e-vii), (e-viii) or (e-ix). More preferably, Hyp^{x} is of formula (e-ii), (e-iii), (e-iv), (e-vii), (e-viii) or (e-ix), even more preferably Hyp^{x} is of formula (e-ii), (e-iii), (e-vii) or (e-viii) and most preferably Hyp^{x} is of formula (e-iii).

Preferrably, the moiety -A²-Hyp¹ is

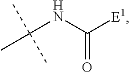

wherein
the dashed line indicates attachment to P; and
E¹ is selected from formulas (e-i) to (e-ix).

Preferrably, the moiety Hyp²-A³- is

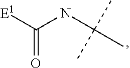

wherein
the dashed line indicates attachment to P; and
E¹ is selected from formulas (e-i) to (e-ix).

Preferably, the moiety -A⁴-Hyp³ is

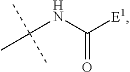

wherein
the dashed line indicates attachment to P; and
E¹ is selected from formulas (e-i) to (e-ix).

Preferably, the moiety -A⁵-Hyp⁴ is

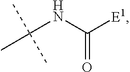

wherein
the dashed line indicates attachment to P¹; and
E¹ is selected from formulas (e-i) to (e-ix).

More preferably, the backbone reagent is of formula (IIIa) and B is of formula (a-xiv).

Even more preferably, the backbone reagent is of formula (IIIa), B is of formula (a-xiv), x1 and x2 are 0, and A¹ is —O—.

Even more preferably, the backbone reagent is of formula (IIIa), B is of formula (a-xiv), A¹ is —O—, and P is of formula (c-i).

Even more preferably, the backbone reagent is of formula (IIIa), B is of formula (a-xiv), x1 and x2 are 0, A¹ is —O—, and P is of formula (c-i).

Most preferably, the backbone reagent has the following formula:

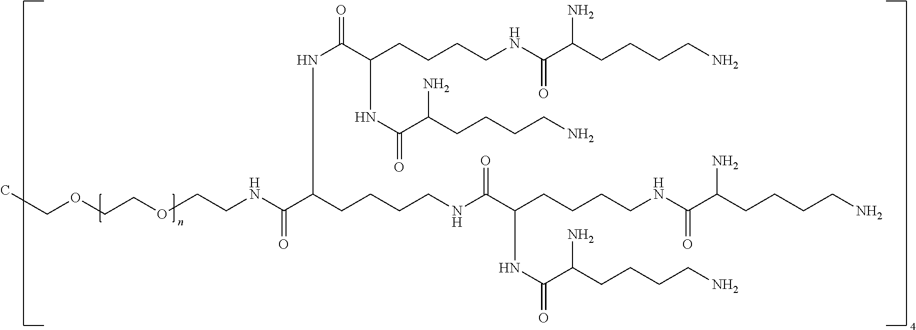

wherein
n ranges from 10 to 40, preferably from 10 to 30, more preferably from 10 to 20.

Equally preferably, n ranges from 20 to 30 and most preferably n is 28.

The Crosslinker Reagent of Step (a-ib)

Preferably, the at least one crosslinker reagent of step (a-ib) comprises a polymer.

The at least one crosslinker reagent of step (a-ib) comprises one or more polymer(s) selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

Preferably, the at least one crosslinker reagent of step (a-ib) comprises hyaluronic acid or PEG.

In one preferred embodiment, the at least one crosslinker reagent of step (a-ib) comprises hyaluronic acid. Preferably, such hyaluronic acid-comprising crosslinker reagent of step (a-ib) comprises at least 70% hyaluronic acid, more preferably at least 80% hyaluronic acid and most preferably at least 90% hyaluronic acid and further comprises
(i) at least two carbonyloxy groups (—(C═O)—O— or —O—(C═O)—), and additionally
(ii) at least two functional end groups selected from the group consisting of activated ester groups, activated carbamate groups, activated carbonate groups, activated thiocarbonate groups, amine groups and thiol groups.

In another preferred embodiment, the at least one crosslinker reagent of step (a-ib) comprises PEG. Preferably, such PEG-comprising crosslinker reagent of step (a-ib) comprises at least 70% PEG, more preferably at least 80% PEG and most preferably at least 90% PEG and further comprises
(i) at least two carbonyloxy groups (—(C═O)—O— or —O—(C═O)—), and additionally
(ii) at least two functional end groups selected from the group consisting of activated ester groups, activated carbamate groups, activated carbonate groups, activated thiocarbonate groups, amine groups and thiol groups.

The at least one crosslinker reagent preferably comprises at least two carbonyloxy groups (—(C═O)—O— or —O—(C═O)—), which are biodegradable linkages. These biodegradable linkages render the hydrogel biodegradable which is advantageous. In addition, the at least one crosslinker reagent comprises at least two functional end groups which during the polymerization of step (a-ii) react with the functional groups A^{x0} of the at least one backbone reagent of step (a-ia).

The crosslinker reagent has a molecular weight ranging from 0.2 to 40 kDa, more preferably ranging from 0.5 to 30 kDa, even more preferably ranging from 0.5 to 20 kDa, even more preferably ranging from 0.5 to 15 kDa and most preferably ranging from 1 to 10 kDa.

Preferably, the reaction of a functional end group of a crosslinker reagent with a functional group A^{x0} of a backbone reagent leads to the formation of an amide linkage between a backbone moiety and a crosslinker moiety, i.e. a backbone moiety and a crosslinker moiety are preferably connected through an amide linkage.

In one preferred embodiment, the crosslinker reagent is a compound of formula (IV-I):

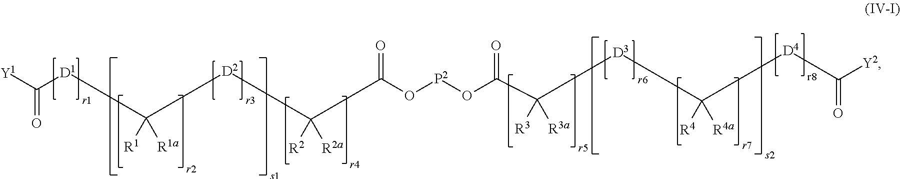

wherein
each D¹, D², D³ and D⁴ are identical or different and each is independently of the others selected from the group comprising —O—, —NR⁵—, —S— and —CR⁶R^{6a}—;
each R¹, R^{1a}, R², R^{2a}, R³, R^{3a}, R⁴, R^{4a}, R⁶ and R^{6a} are identical or different and each is independently of the others selected from the group comprising —H, —OR⁷, —NR⁷R^{7a}, —SR⁷ and C₁₋₆alkyl; optionally, each of the pair(s) R¹/R², R³/R⁴, R^{1a}/R^{2a}, and R^{3a}/R^{4a} may independently form a chemical bond and/or each of the pairs R¹/R^{1a}, R²/R^{2a}, R³/R^{3a}, R⁴/R^{4a}, R⁶/R^{6a}, R¹/R², R³/R⁴, R^{1a}/R^{2a}, and R^{3a}/R^{4a} are independently of each other joined together with the atom to which they are attached to form a C₃₋₈cycloalkyl or to form a ring A or are joined together with the atom to which they are attached to form a 4- to 7-membered heterocyclyl or 8- to 11-membered heterobicyclyl or adamantyl;
each R⁵ is independently selected from —H and C₁₋₆alkyl; optionally, each of the pair(s) R¹/R⁵, R²/R⁵, R³/R⁵, R⁴/R⁵ and R⁵/R⁶ may independently form a chemical bond and/or are joined together with the atom to which they are attached to form a 4- to 7-membered heterocyclyl or 8- to 11-membered heterobicyclyl;
each R⁷, R^{7a} is independently selected from H and C₁₋₆alkyl;
A is selected from the group consisting of indenyl, indanyl and tetralinyl;
P² is

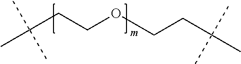

m ranges from 120 to 920, preferably from 120 to 460 and more preferably from 120 to 230,
r1, r2, r7, r8 are independently 0 or 1;
r3, r6 are independently 0, 1, 2, 3, or 4;
r4, r5 are independently 1, 2, 3, 4, 5, 6, 7, 8, 9 or 10;
s1, s2 are independently 1, 2, 3, 4, 5 or 6;
Y¹, Y² are identical or different and each is independently of the other selected from formulas (f-i) to (f-vi):

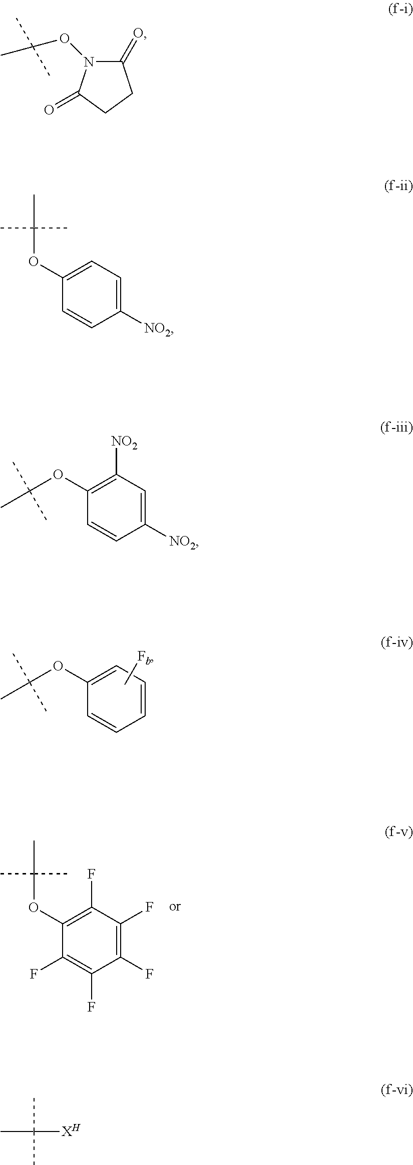

wherein
the dashed lines indicate attachment to the rest of the molecule,
b is 1, 2, 3 or 4
X^{H} is Cl, Br, I, or F.

Preferably, the crosslinker reagent is a compound of formula (IV-II):

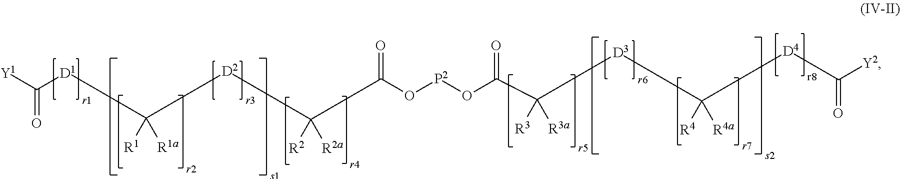

wherein
D¹, D², D³ and D⁴ are identical or different and each is independently of the others selected from the group consisting of O, NR⁵, S and CR⁵R^{5 a};
R¹, R^{1a}, R², R^{2a}, R³, R^{3a}, R⁴, R^{4a}, R⁵ and R^{5a} are identical or different and each is independently of the others selected from the group consisting of H and C₁₋₆alkyl; optionally, one or more of the pair(s) R¹/R^{1a}, R²/R^{2a}, R³/R^{3a}, R⁴/R^{4a}, R¹/R², R³/R⁴, R^{1a}/R^{2a}, and R^{3a}/R^{4a} form a chemical bond or are joined together with the atom to which they are attached to form a C₃₋₈cycloalkyl or to form a ring A or are joined together with the atom to which they are attached to form a 4- to 7-membered heterocyclyl or 8- to 11-membered heterobicyclyl or adamantyl;
A is phenyl, naphthyl, indenyl, indanyl or tetralinyl;
P² is

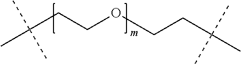

m ranges from 5 to 920, preferably from 5 to 460 and more preferably from 40 to 230;
r1, r2, r7, r8 are independently 0 or 1;
r3, r6 are independently 0, 1, 2, 3, or 4;
r4, r5 are independently 1, 2, 3, 4, 5, 6, 7, 8, 9 or 10;
s1, s2 are independently 1, 2, 3, 4, 5 or 6;
Y¹, Y² are identical or different and each is independently of the other selected from formulas (f-i) to (f-vi):

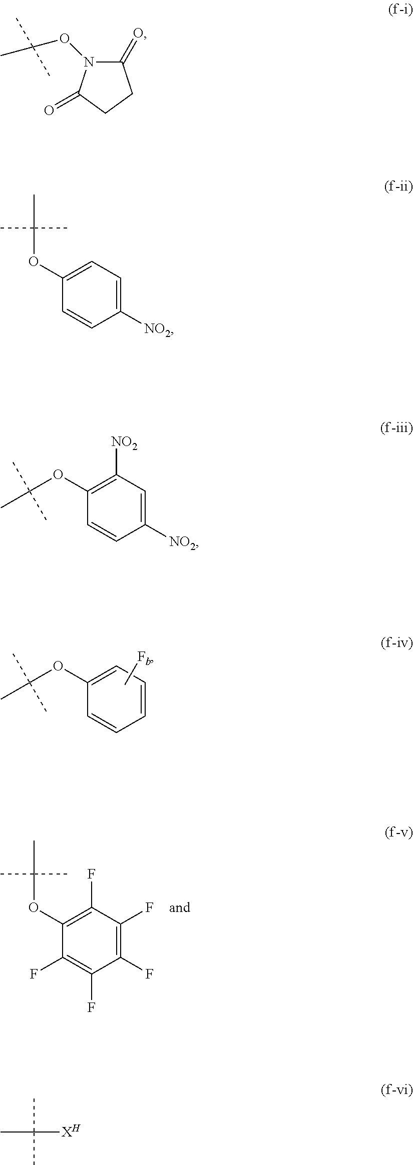

wherein
the dashed lines indicate attachment to the rest of the molecule,
b is 1, 2, 3 or 4
X^{H} is Cl, Br, I, or F.

Preferably, Y¹ and Y² of formula (IV-I) and (IV-II) are of formula (f-i), (f-ii) or (f-v). More preferably, Y¹ and Y² of formula (IV-I) and (IV-II) are of formula (f-i) or (f-ii) and most preferably, Y¹ and Y² of formula (IV-I) and (IV-II) are of formula (f-i).

Preferably, both moieties Y¹ and Y² of formula (IV-I) and (IV-II) have the same structure. More preferably, both Y¹ and Y² are of formula (f-i).

It is understood that the moieties

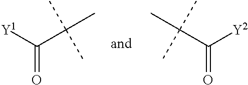

of formula (IV-I) and (IV-II) represent the at least two functional end groups.

Preferably, r1 and r8 of formula (IV-I) and (IV-II) are both 0.

Preferably, r1, r8, s1 and s2 of formula (IV-I) and (IV-II) are all 0.

Preferably, one or more of the pair(s) R¹/R^{1a}, R²/R^{2a}, R³/R^{3a}, R⁴/R^{4a}, R¹/R², R³/R⁴, R^{1a}/R^{2a}, and R^{3a}/R^{4a} of formula (IV-I) and (IV-II) form a chemical bond or are joined together with the atom to which they are attached to form a C₃₋₈cycloalkyl or a ring A.

Preferably, one or more of the pair(s) R¹/R², R^{1a}/R^{2a}, R³/R⁴, R^{3a}/R^{4a} of formula (IV-I) and (IV-II) are joined together with the atom to which they are attached to form a 4- to 7-membered heterocyclyl or 8- to 11-membered heterobicyclyl.

Preferably, the crosslinker reagent of formula (IV-I) and (IV-II) is symmetric, i.e. the moiety

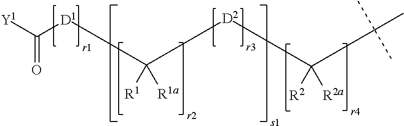

has the same structure as the moiety

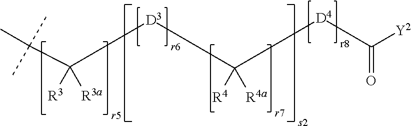

Preferred crosslinker reagents are of formula (g-i) to (g-liv):

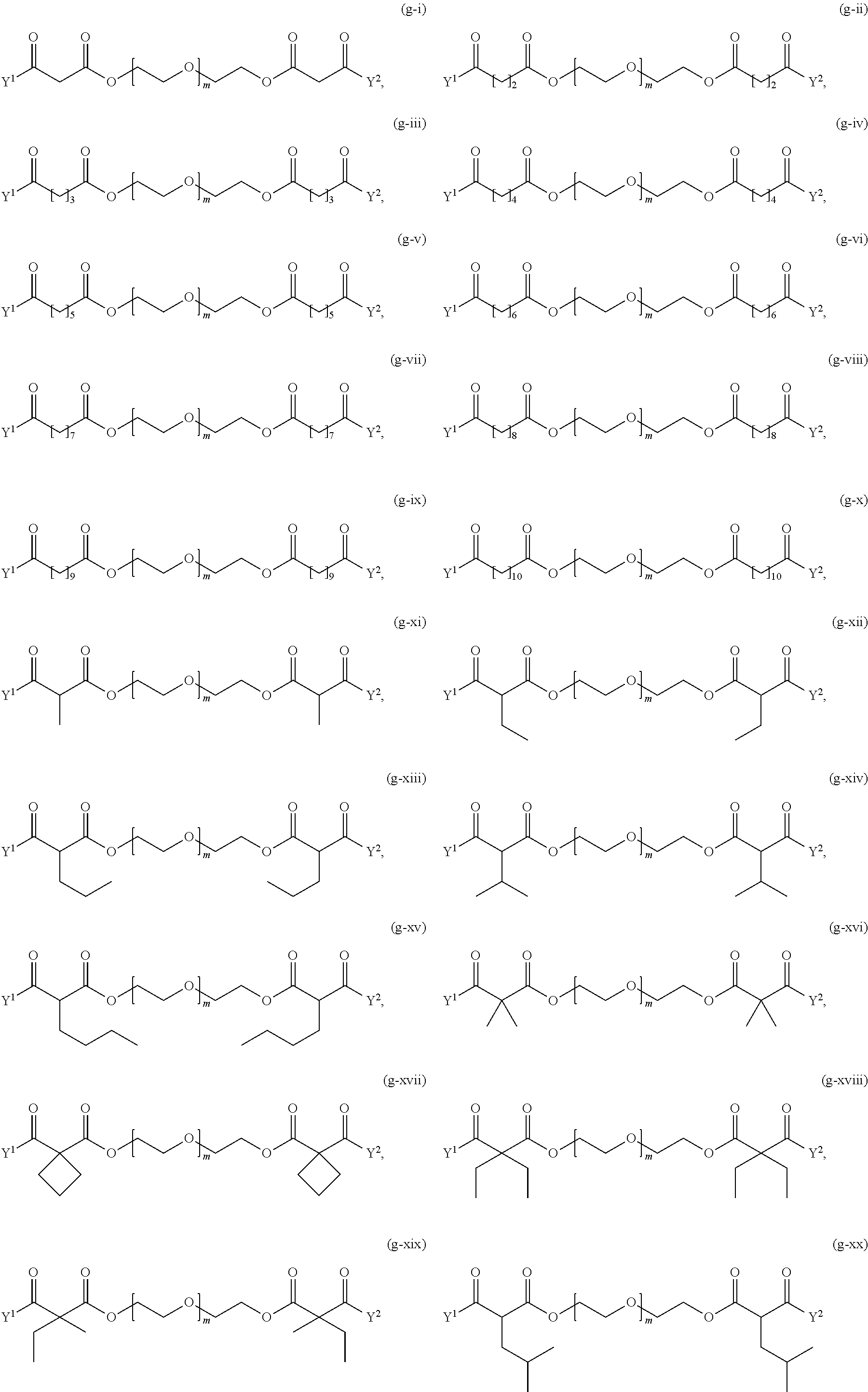

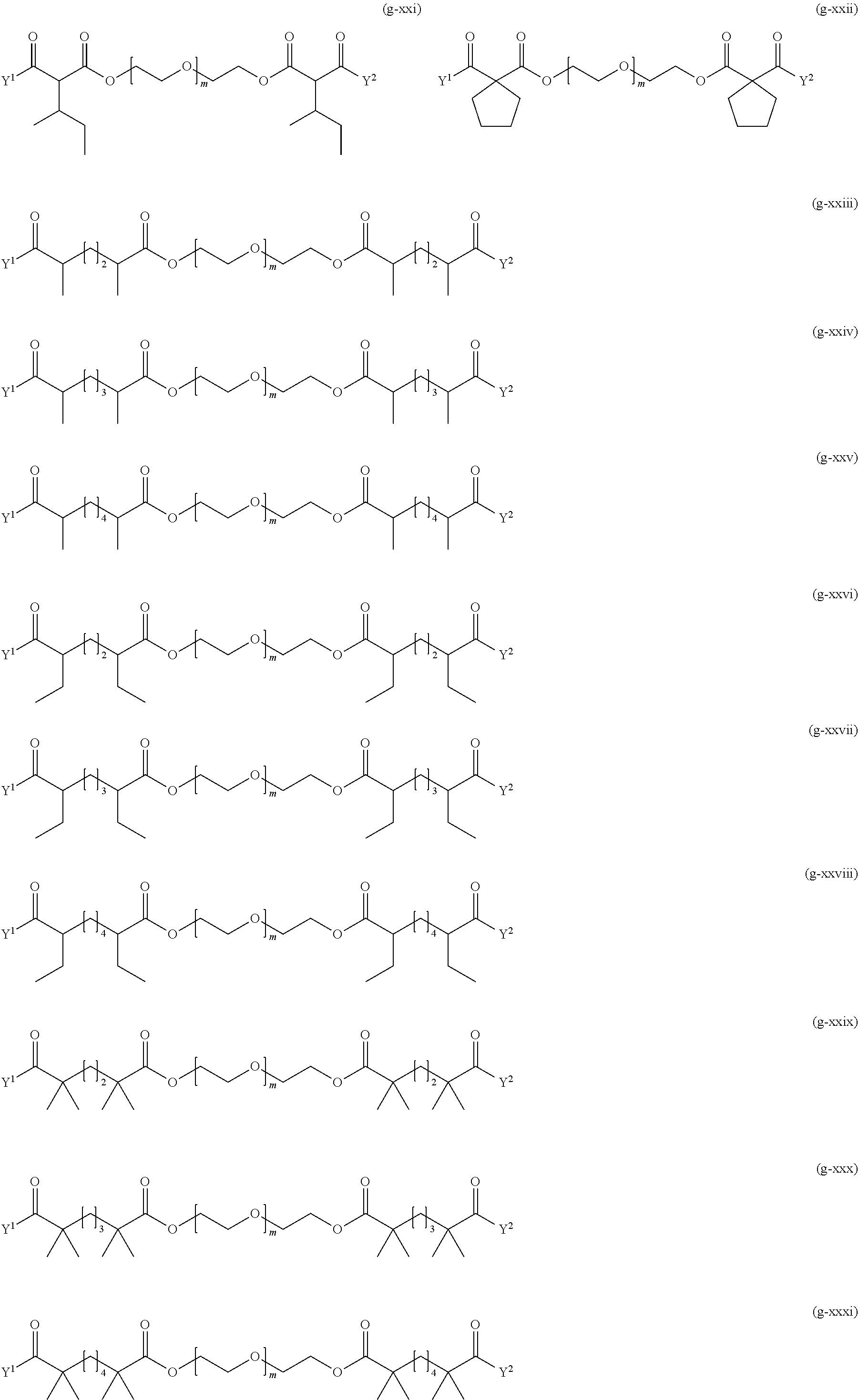

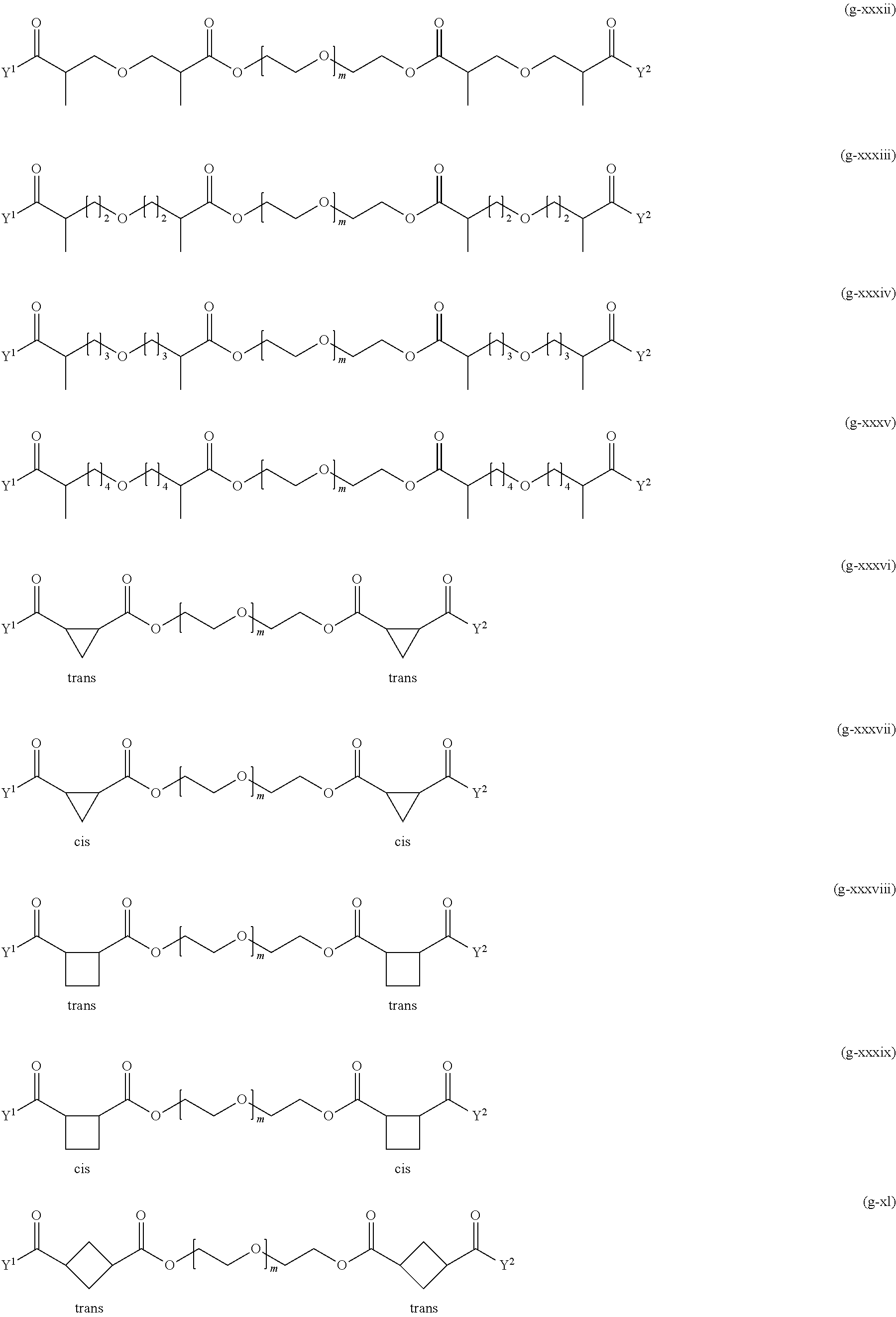

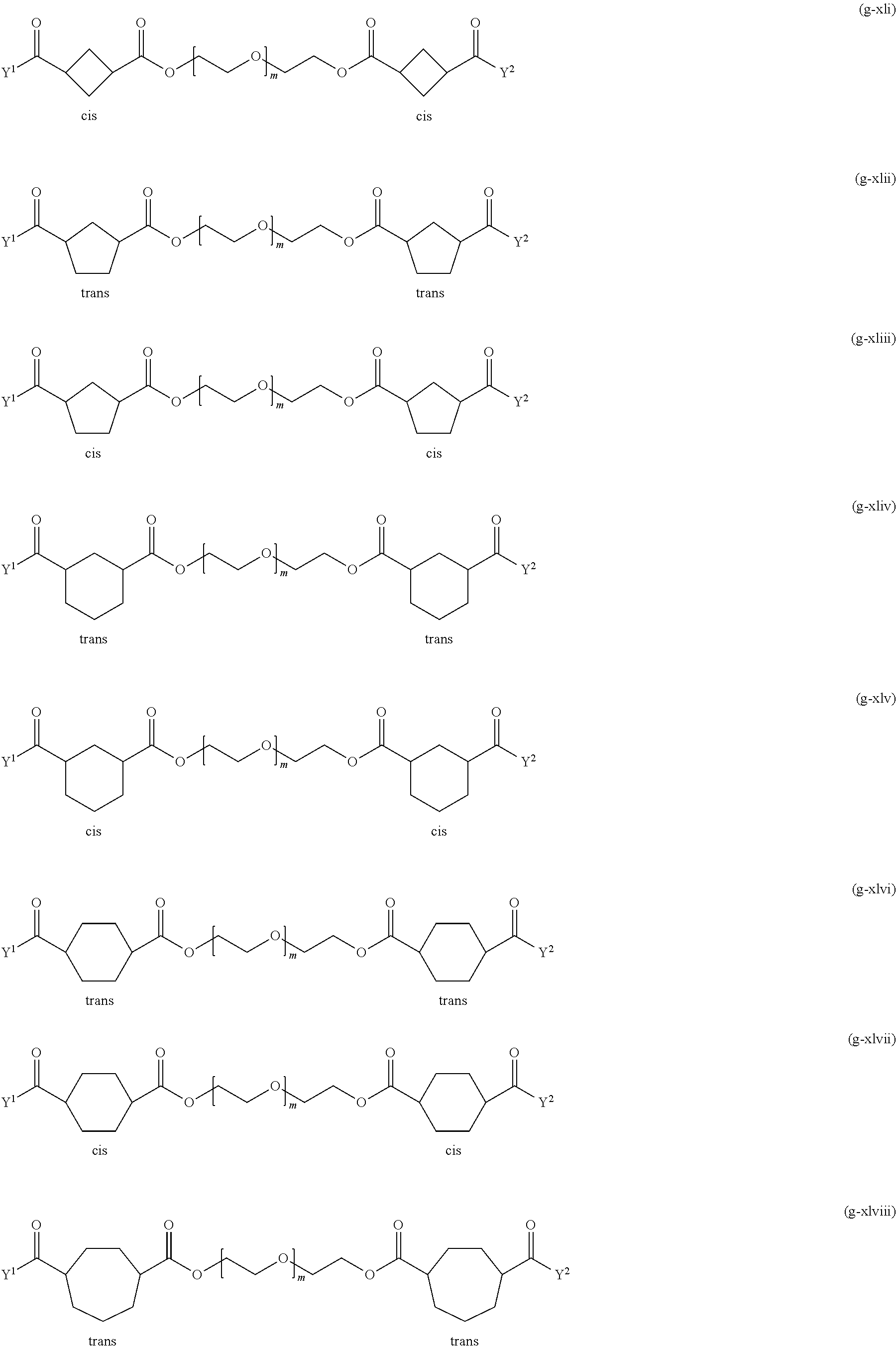

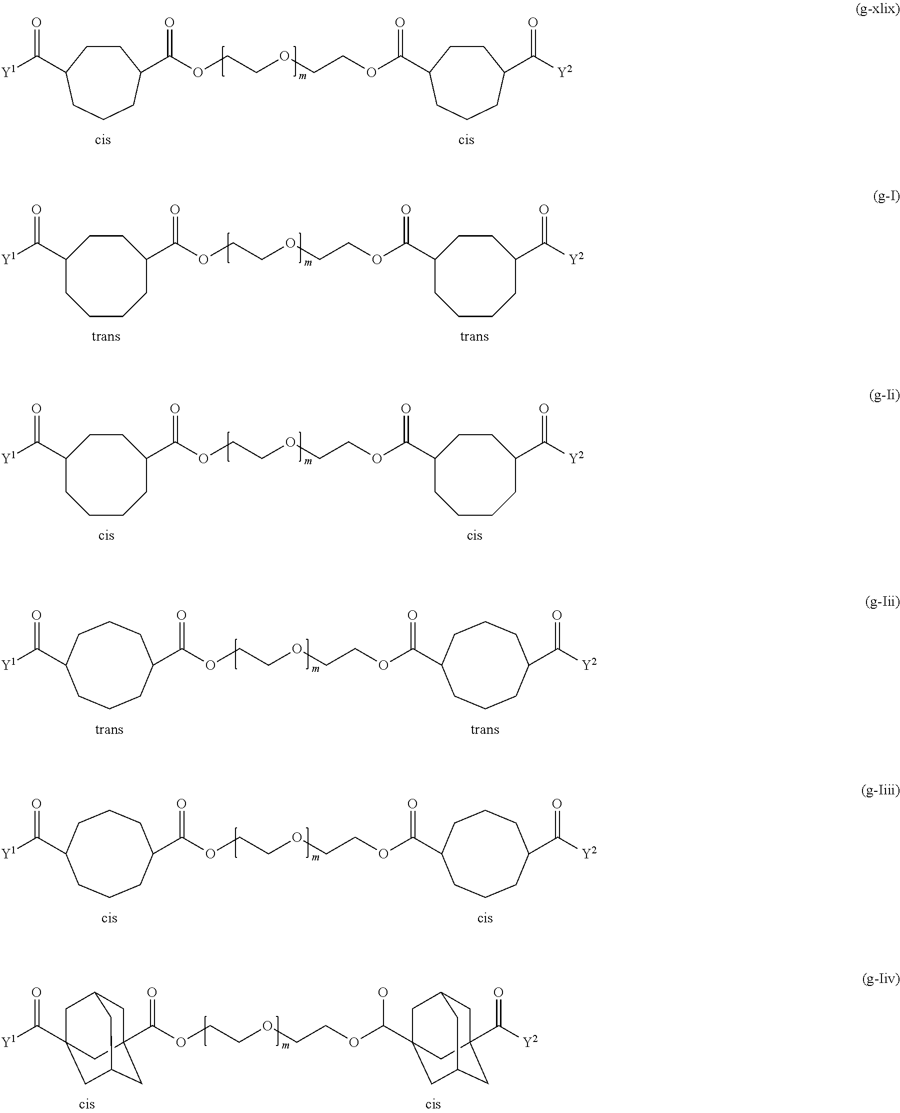

wherein
each crosslinker reagent may be in the form of its racemic mixture, where applicable; and
m, Y¹, Y² are defined as above.

Even more preferred crosslinker reagents are of formula (ga-1) to (ga-54):

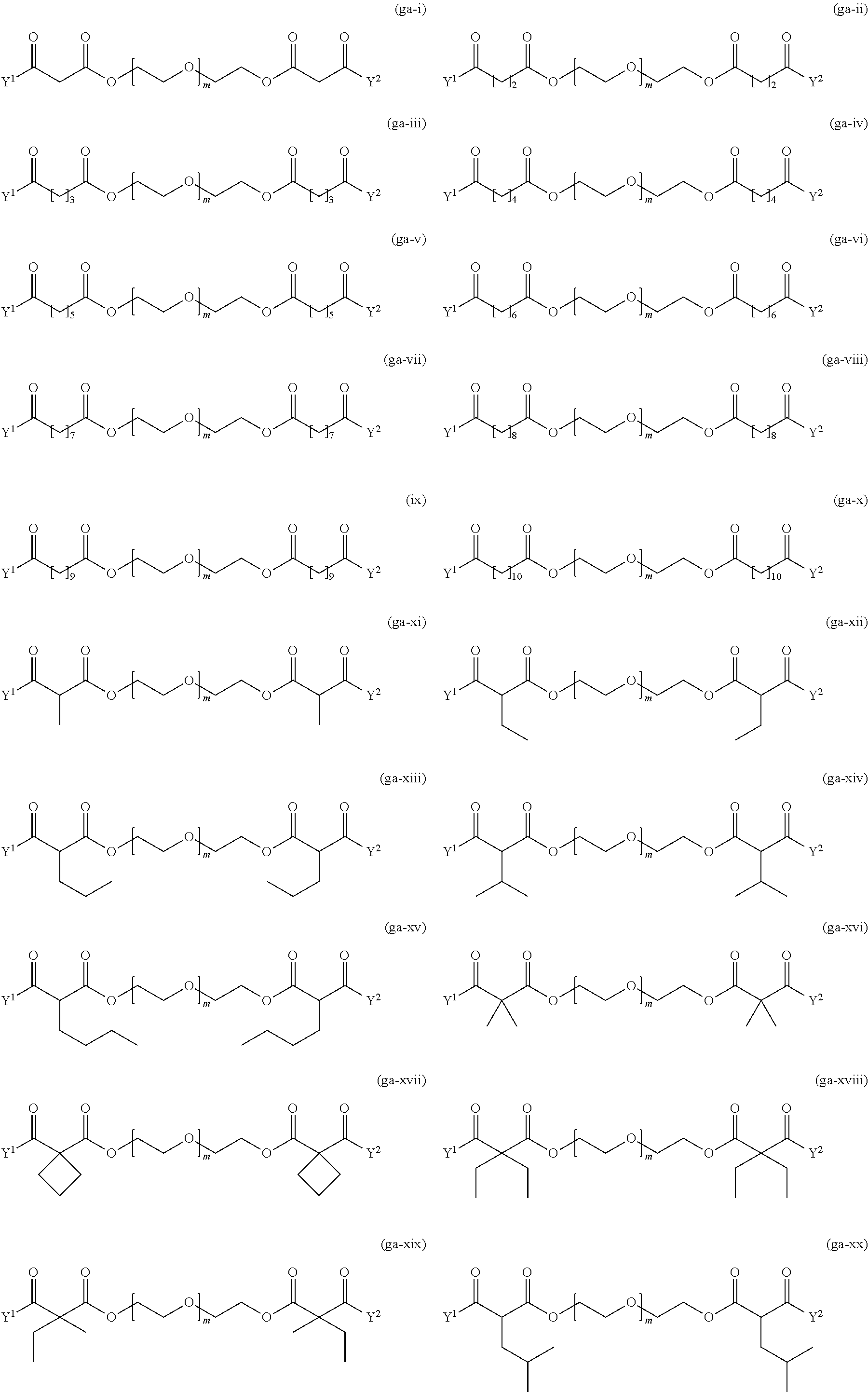

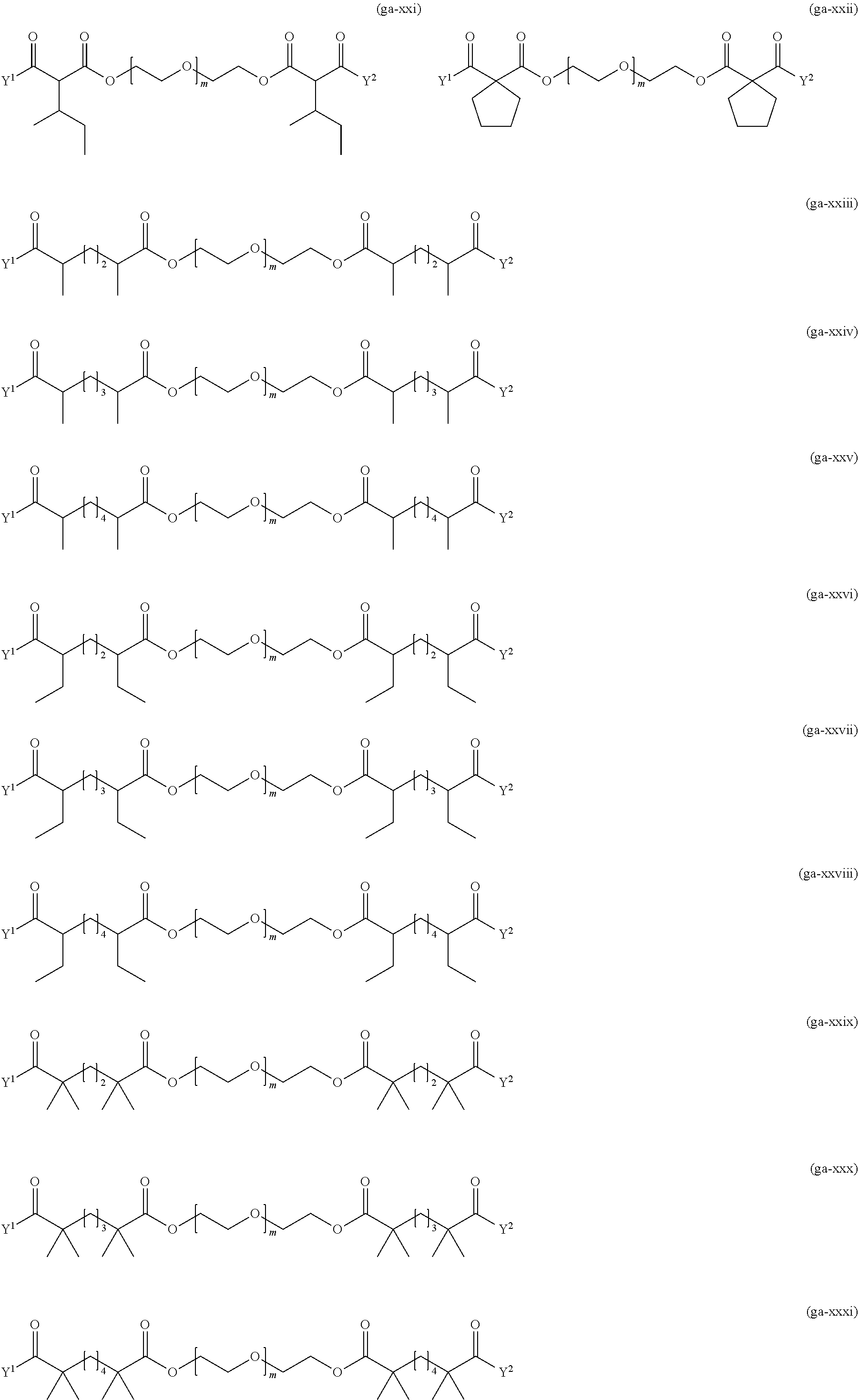

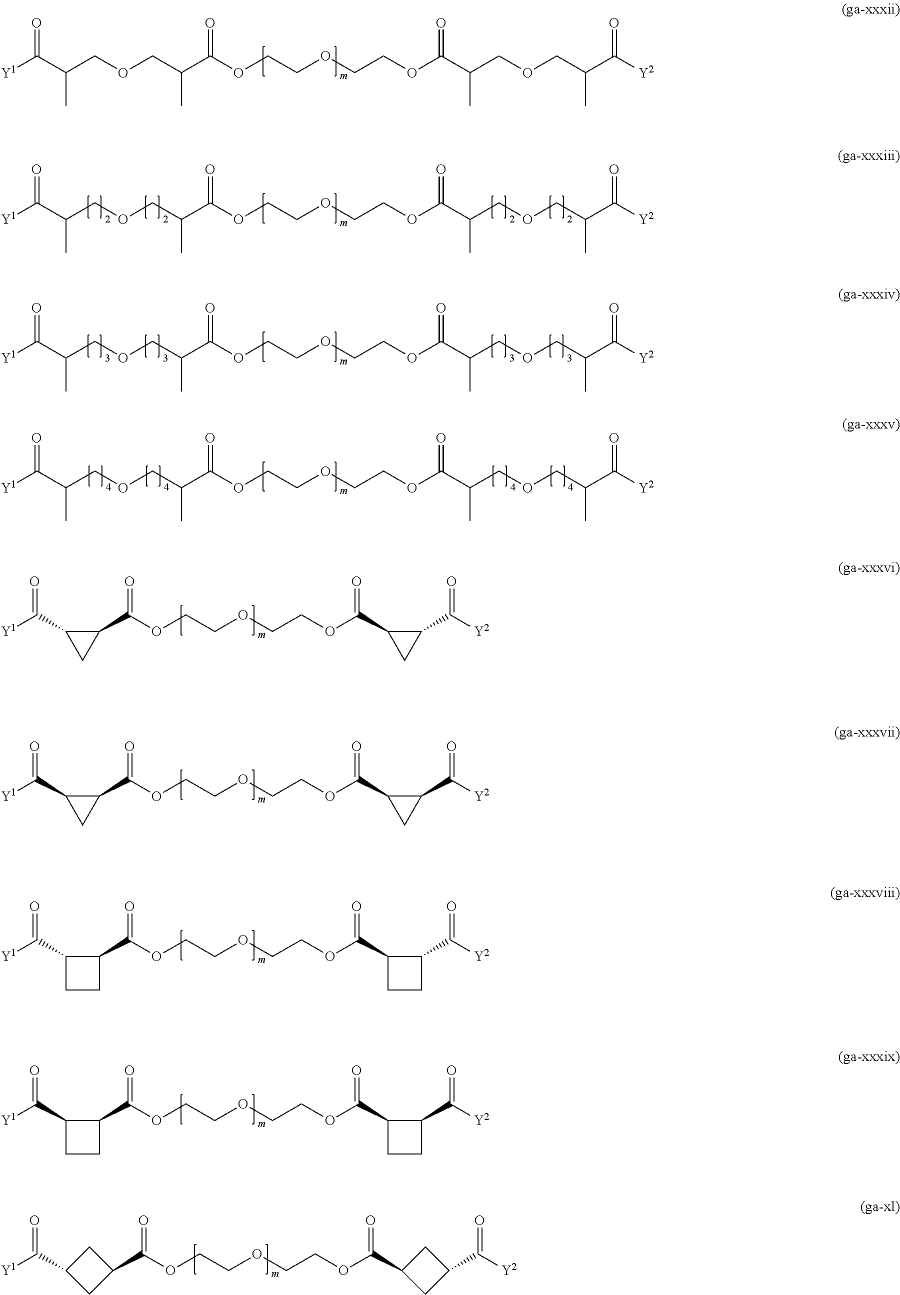

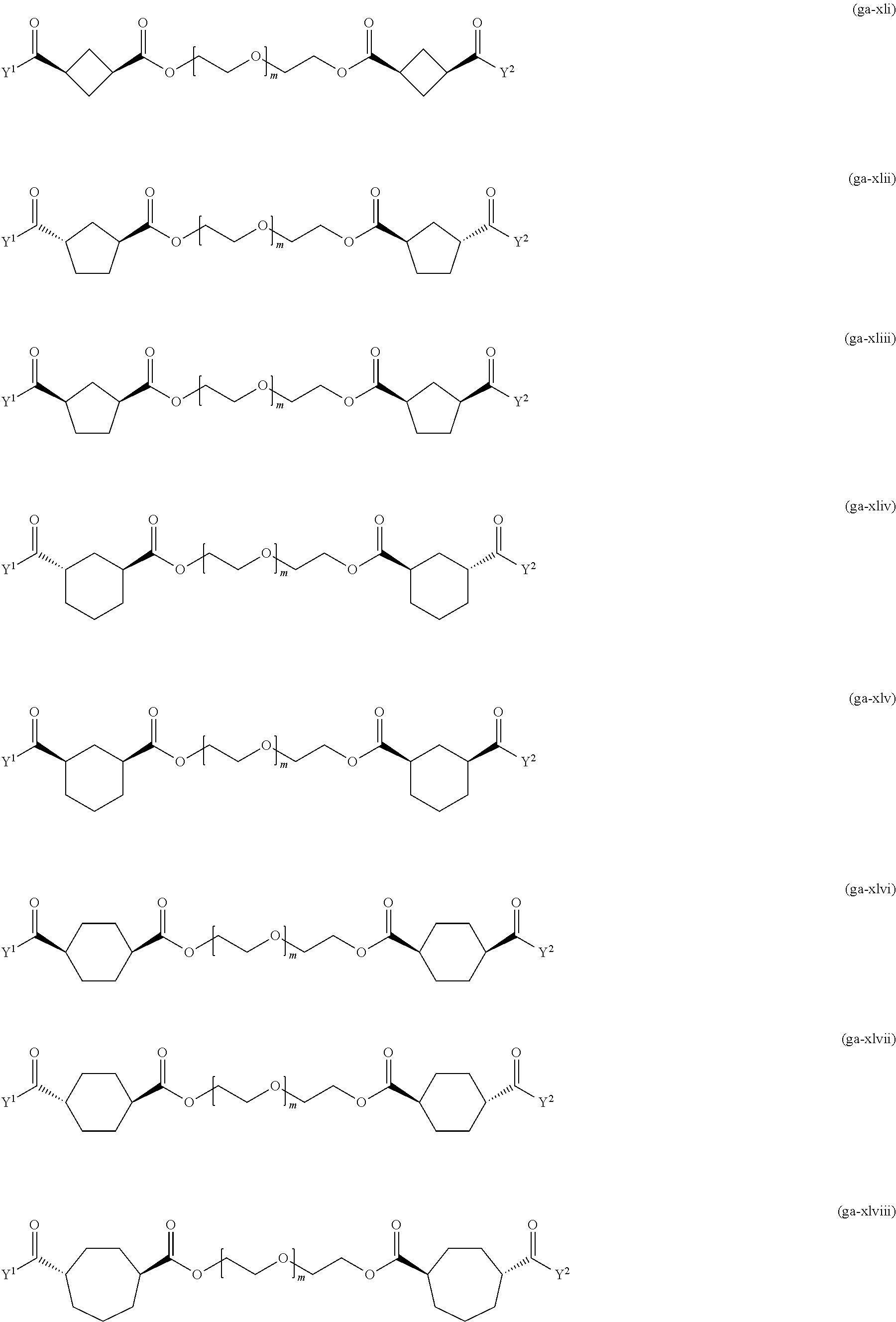

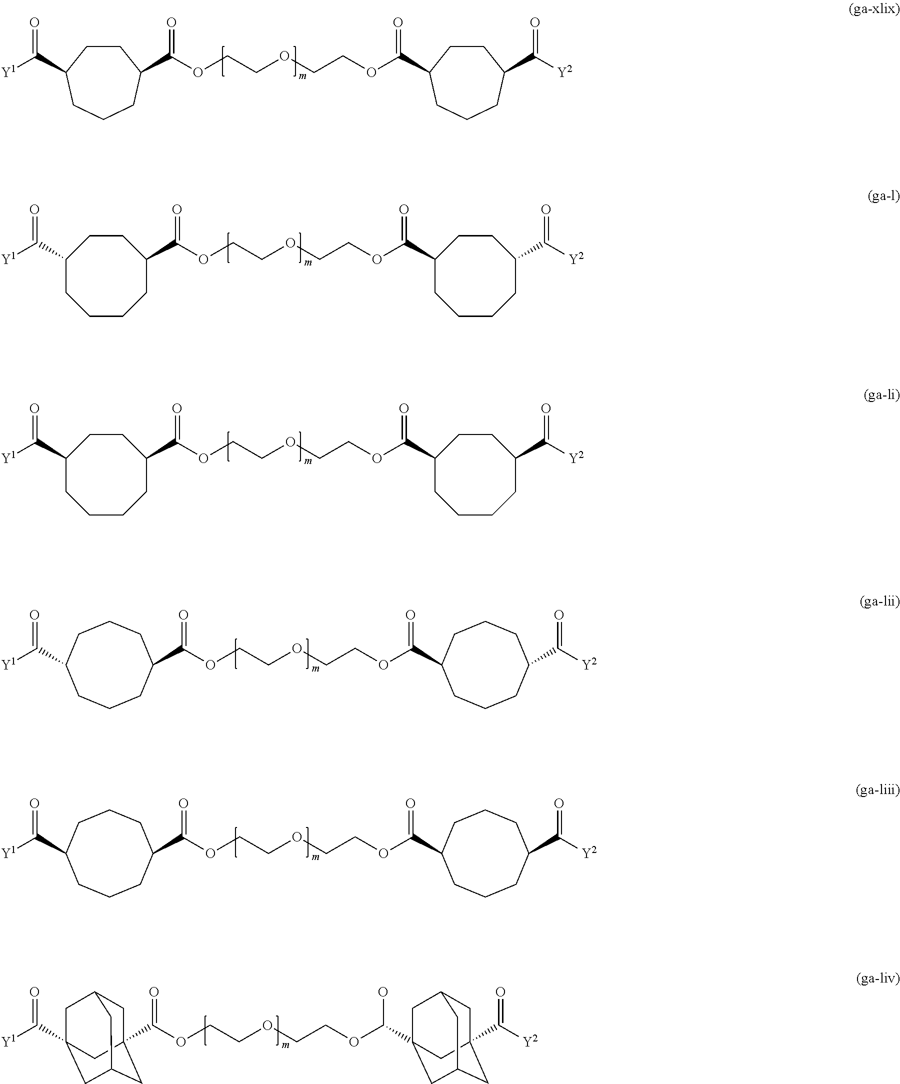

wherein
each crosslinker reagent may be in the form of its racemic mixture, where applicable; and
m, Y¹ and Y² are defined as above.

It was surprisingly found that the use of crosslinker reagents with branches, i.e. residues other than H, at the alpha carbon of the carbonyloxy group lead to the formation of hydrogels which are more resistant against enzymatic degradation, such as degradation through esterases.

Similarly, it was surprisingly found that the fewer atoms there are between the (C═O) of a carbonyloxy group and the (C═O) of the adjacent activated ester, activated carbamate, activated carbonate or activated thiocarbamate, the more resistant against degradation the resulting hydrogels are, such as more resistant against degradation through esterases.

In one embodiment crosslinker reagents g-i, g-ii, g-v, g-vi, g-vii, g-viii, g-ix, g-x, g-xi, g-xii, g-xiii, g-xiv, g-xv, g-xvi, g-xvii, g-xviii, g-xix, g-xx, g-xxi, g-xxii, g-xxiii, g-xxiv, g-xxv, g-xxvi, g-xxvii, g-xxviii, g-xxix, g-xxx, g-xxxi, g-xxxii, g-xxxiii, g-xxxiv, g-xxxv, g-xxxvi, g-xxvii, g-xxxviii, g-xxxix, g-xl, g-xli, g-xlii, g-xliii, g-xliv, g-xlv, g-xlvi, g-xlvii, g-xlviii, g-xlix, g-l, g-li, g-lii, g-liii and g-liv are preferred crosslinker reagents. More preferably, the at least one crosslinker reagent is of formula g-v, g-vi, g-vii, g-viii, g-ix, g-x, g-xiv, g-xxxii, g-xxxiii, g-xliii, g-xliv, g-xlv or g-xlvi, and even more preferably, the at least one crosslinker reagent is of formula g-v, g-vi, g-ix or g-xiv. Most preferably, the at least one crosslinker reagent is of formula g-xiv.

In another embodiment crosslinker reagents ga-i, ga-ii, ga-v, ga-vi, ga-vii, ga-viii, ga-ix, ga-x, ga-xi, ga-xii, ga-xiii, ga-xiv, ga-xv, ga-xvi, ga-xvii, ga-xviii, ga-xix, ga-xx, ga-xxi, ga-xxii, ga-xxiii, ga-xxiv, ga-xxv, ga-xxvi, ga-xxvii, ga-xxviii, ga-xxix, ga-xxx, ga-xxxi, ga-xxxii, ga-xxxiii, ga-xxxiv, ga-xxxv, ga-xxxvi, ga-xxvii, ga-xxxviii, ga-xxxix, ga-xl, ga-xli, ga-xlii, ga-xliii, ga-xliv, ga-xlv, ga-xlvi, ga-xlvii, ga-xlviii, ga-xlix, ga-i, ga-li, ga-lii, ga-liii and ga-liv are preferred crosslinker reagents. More preferably, the at least one crosslinker reagent is of formula ga-v, ga-vi, ga-vii, ga-viii, ga-ix, ga-x, ga-xiv, ga-xxxii, ga-xxxiii, ga-xliii, ga-xliv, ga-xlv or ga-xlvi, and even more preferably, the at least one crosslinker reagent is of formula ga-v, ga-vi, ga-ix or ga-xiv. Most preferably, the at least one crosslinker reagent is of formula ga-xiv.

The preferred embodiments of the compound of formula of formula (IV-I) and (IV-II) as mentioned above apply accordingly to the preferred compounds of formulas (g-i) to (g-liv).

The hydrogel resulting from step (a-ii) preferably contains from 0.01 to 1.2 mmol/g primary amine groups (—NH₂), more preferably from 0.02 to 1.0 mmol/g primary amine groups, even more preferably from 0.02 to 0.5 mmol/g primary amine groups and most preferably from 0.05 to 0.3 mmol/g primary amine groups, if it is to be used as a carrier in a hydrogel-linked prodrug of a protein drug. If the hydrogel of the present invention is to be used as a carrier in a hydrogel-linked prodrug of a small molecule, it preferably contains from 0.01 to 2 mmol/g primary amine groups, more preferably from 0.02 to 1.8 mmol/g primary amine groups, even most preferably from 0.05 to 1.5 mmol/g primary amine groups.

More preferably, the hydrogel resulting from step (a-ii) preferably contains from 0.01 to 1.2 mmol/g primary amine groups (—NH₂), more preferably from 0.02 to 1.0 mmol/g primary amine groups, even more preferably from 0.02 to 0.5 mmol/g primary amine groups and most preferably from 0.05 to to 0.3 mmol/g primary amine groups.

The term “X mmol/g primary amine groups” means that 1 g of dry hydrogel comprises X mmol primary amine groups. Measurement of the amine content of the hydrogel is carried out according to Gude et al. (Letters in Peptide Science, 2002, 9(4): 203-206, which is incorpated by reference in its entirety) and is also described in detail in the Examples section.

Preferably, the term “dry” as used herein means having a residual water content of a maximum of 10%, preferably less than 5% and more preferably less than 2% (determined according to Karl Fischer). The preferred method of drying is lyophilization.

Polymerization of Step (a-ii)

In one embodiment the polymerization in step (a-ii) is initiated by adding a base. Preferably, the base is a non-nucleophilic base soluble in alkanes, more preferably the base is selected from the group consisting of N,N,N′,N′-tetramethylethylene diamine (TMEDA), 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, triethylamine, diisopropylethylamine (DIPEA), trimethylamine, N,N-dimethylethylamine, N,N,N′,N′-tetramethyl-1,6-hexanediamine, N,N,N′,N″,N″-pentamethyldiethylenetriamine, 1,8-diazabicyclo[5.4.0]undec-7-ene, 1,5-diazabicyclo[4.3.0]non-5-ene, and hexamethylenetetramine. Even more preferably, the base is selected from TMEDA, 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, 1,8-diazabicyclo[5.4.0]undec-7-ene, 1,5-diazabicyclo[4.3.0]non-5-ene, and hexamethylenetetramine. Most preferably, the base is TMEDA.

The base to initiate the polymerization in step (a-ii) is added preferably in an amount of 1 to 500 equivalents per activated functional end group in the mixture, more preferably in an amount of 5 to 50 equivalents, even more preferably in an amount of 5 to 25 equivalents and most preferably in an amount of 10 equivalents.

Preferably, the polymerization of step (a-ii) is a condensation reaction, which preferably occurs under continuous stirring of the mixture of step (a). Preferably, the tip speed (tip speed=π×stirrer rotational speed x stirrer diameter) ranges from 0.2 to 10 meter per second (m/s), more preferably from 0.5 to 4 m/s and most preferably from 1 to 2 m/s.

In a preferred embodiment of step (a-ii), the polymerization reaction is carried out in a cylindrical vessel equipped with baffles. The diameter to height ratio of the vessel preferably ranges from 4:1 to 1:2, more preferably the diameter to height ratio of the vessel ranges from 2:1 to 1:1.

Preferably, the reaction vessel is equipped with an axial flow stirrer selected from the group consisting of pitched blade stirrers, marine type propellers, and Lightnin A-310. More preferably, the stirrer is a pitched blade stirrer.

Step (a-ii) can be performed in a broad temperature range, preferably at a temperature from −10° C. to 100° C., more preferably at a temperature of 0° C. to 80° C., even more preferably at a temperature of 10° C. to 50° C. and most preferably at ambient temperature.

“Ambient temperature” refers to the temperature present in a typical laboratory environment and preferably means a temperature ranging from 17 to 25° C.

In one preferred embodiment the polymerization in step (a-ii) occurs in a suspension polymerization, in which case the mixture of step (a-ii) further comprises a first solvent and at least a second solvent, which second solvent is immiscible in the first solvent.

Said first solvent is preferably selected from the group consisting of dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol, water and mixtures thereof.

The at least one backbone reagent and at least one crosslinker reagent are dissolved in the first solvent, i.e. the disperse phase of the suspension polymerization. In one embodiment the at least one backbone reagent and the at least one crosslinker reagent are dissolved separately, i.e. in different containers, using either the same or different solvent and preferably using the same solvent for both reagents. In another embodiment, the at least one backbone reagent and the at least one crosslinker reagent are dissolved together, i.e. in the same container and using the same solvent.

A suitable solvent for the at least one backbone reagent is an organic solvent. Preferably, the solvent is selected from the group consisting of dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol, water and mixtures thereof. More preferably, the backbone reagent is dissolved in a solvent selected from acetonitrile, dimethyl sulfoxide, methanol and mixtures thereof. Most preferably, the backbone reagent is dissolved in dimethylsulfoxide.

In one embodiment the at least one backbone reagent is dissolved in the solvent in a concentration ranging from 1 to 300 mg/ml, more preferably from 5 to 60 mg/ml and most preferably from 10 to 40 mg/ml.

A suitable solvent for the at least one crosslinker reagent is an organic solvent. Preferably, the solvent is selected from the group consisting of dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol, water and mixtures thereof. More preferably, the crosslinker reagent is dissolved in a solvent selected from dimethylformamide, acetonitrile, dimethyl sulfoxide, methanol and mixtures thereof. Most preferably, the crosslinker reagent is dissolved in dimethylsulfoxide.

In one embodiment the at least one crosslinker reagent is dissolved in the solvent in a concentration ranging from 5 to 500 mg/ml, more preferably from 25 to 300 mg/ml and most preferably from 50 to 200 mg/ml.

The at least one backbone reagent and the at least one crosslinker reagent are mixed in a weight ratio ranging from 1:99 to 99:1, e.g. in a ratio ranging from 2:98 to 90:10, in a weight ratio ranging from 3:97 to 88:12, in a weight ratio ranging from 3:96 to 85:15, in a weight ratio ranging from 2:98 to 90:10 and in a weight ratio ranging from 5:95 to 80:20; particularly preferred in a weight ratio from 5:95 to 80:20, wherein the first number refers to the at least one backbone reagent and the second number to the at least one crosslinker reagent.

The ratios are selected such that the mixture of step (a-i) comprises a molar excess of functional groups A^{x0} from the at least one backbone reagent compared to the functional end groups of the at least one crosslinker reagent. Consequently, the hydrogel resulting from the process of the present invention has free functional groups A^{x0} groups which can be used to couple other moieties to the hydrogel, such as spacer moieties and/or reversible prodrug linker moieties.

The at least one second solvent, i.e. the continuous phase of the suspension polymerization, is preferably an organic solvent, more preferably an organic solvent selected from the group consisting of linear, branched or cyclic C₅₋₃₀ alkanes; linear, branched or cyclic C₅₋₃₀ alkenes; linear, branched or cyclic C₅₋₃₀ alkynes; linear or cyclic poly(dimethylsiloxanes); aromatic C₆₋₂₀ hydrocarbons; and mixtures thereof. Even more preferably, the at least second solvent is selected from the group consisting of linear, branched or cyclic C₅₋₁₆ alkanes; toluene; xylene; mesitylene; hexamethyldisiloxane; and mixtures thereof. Most preferably, the at least second solvent is a linear C₇₋₁₁ alkane, such as heptane, octane, nonane, decane or undecane.

Preferably, the mixture of step (a-i) further comprises a detergent. Preferred detergents are Cithrol DPHS, Hypermer 70A, Hypermer B246, Hypermer 1599A, Hypermer 2296, and Hypermer 1083.

Preferably, the detergent has a concentration of 0.1 g to 100 g per 1 L total mixture, i.e. disperse phase and continous phase together. More preferably, the detergent has a concentration of 0.5 g to 10 g per 1 L total mixture, and most preferably, the detergent has a concentration of 0.5 g to 5 g per 1 L total mixture.

Preferably, the mixture of step (a-i) is an emulsion.

Optional Step (a-iii)

Optional step (a-iii) comprises one or more of the following steps:

(a-iiia) removing excess liquid from the polymerization reaction,

(a-iiib) washing the hydrogel to remove solvents used during polymerization,

(a-iiic) transferring the hydrogel into a buffer solution,

(a-iiid) size fractionating/sieving of the hydrogel,

(a-iiie) transferring the hydrogel into a container,

(a-iiif) drying the hydrogel,

(a-iiig) transferring the hydrogel into a specific solvent suitable for sterilization, and/or

(a-iiih) sterilizing the hydrogel, preferably by gamma radiation.

Preferably, the working-up step comprises all of the following steps

(a-iiia) removing excess liquid from the polymerization reaction,

(a-iiib) washing the hydrogel to remove solvents used during polymerization,

(a-iiic) transferring the hydrogel into a buffer solution,

(a-iiid) size fractionating/sieving of the hydrogel,

(a-iiie) transferring the hydrogel into a container,

(a-iiig) transferring the hydrogel into a specific solvent suitable for sterilization, and

(a-iiih) sterilizing the hydrogel, preferably by gamma radiation.

Optional Step (b)

In a preferred embodiment A^{x0} is an amine and A^{x1} is ClSO₂—, R¹(C═O)—, I—, Br—, Cl—, SCN—, CN—, O═C═N—, Y¹—(C═O)—, Y¹—(C═O)—NH—, or Y¹—(C═O)—O—,
wherein
R¹ is H, C₁₋₆alkyl, C₂₋₆alkenyl, C₂₋₆alkynyl, C₃₋₈cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi):

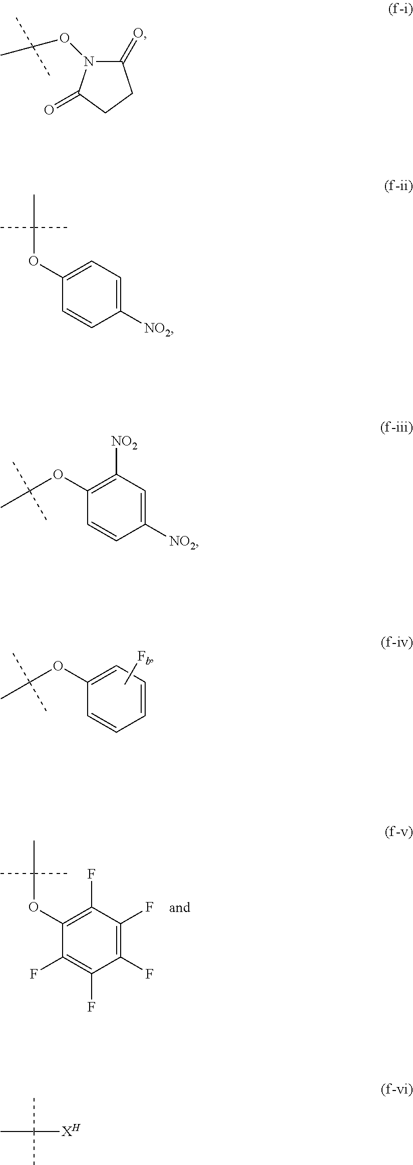

wherein
the dashed lines indicate attachment to the rest of the molecule,
b is 1, 2, 3 or 4,
X^{H} is Cl, Br, I, or F.

In another preferred embodiment A^{x0} is a hydroxyl group (—OH) and A^{x1} is O═C═N—, I—, Br—, SCN—, or Y¹—(C═O)—NH—,
wherein Y¹ is selected from formulas (f-i) to (f-vi):

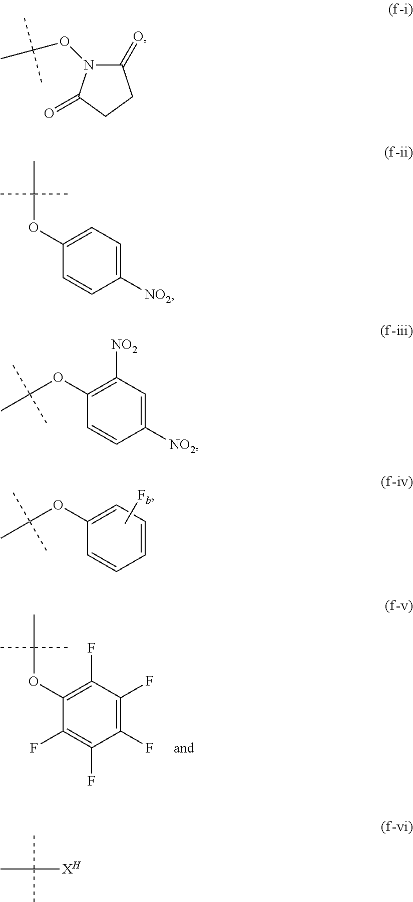

wherein
the dashed lines indicate attachment to the rest of the molecule,
b is 1, 2, 3 or 4,
X^{H} is Cl, Br, I, or F.

In another preferred embodiment A^{x0} is a carboxylic acid (—(C═O)OH) and A^{x1} is a primary amine or secondary amine.

In another preferred embodiment A^{x0} is a maleimide and A^{x1} is a thiol.

More preferably, A^{x0} is an amine and A^{x1} is Y¹—(C═O)—, Y¹—(C═O)—NH—, or Y¹—(C═O)—O— and most preferably A^{x0} is an amine and A^{x1} is Y¹—(C═O)—.

A^{x1} may optionally be present in protected form.

Suitable activating reagents to obtain the activated carboxylic acid are for example N,N′-dicyclohexyl-carbodiimide (DCC), 1-ethyl-3-carbodiimide (EDC), benzotriazol-1-yl-oxytripyrrolidinophosphonium-hexafluorophosphate (PyBOP), bromotripyrrolidinophosphonium hexafluorophosphate (PyBrOP), 1-cyano-2-ethoxy-2-oxoethylidenaminooxy)dimethylamino-morpholino-carbenium-hexafluorophosphate (COMU), 1-hydroxybenzotriazole (HOBT), 1-hydroxy-7-azabenzotriazole (HOAT), O-(6-chlorobenzotriazol-1-yl)-N,N,N′,N′-tetramethyluronium hexafluorophosphate (HCTU), 1-H-benzotriazolium (HBTU), (O-(7-azabenzotriazol-1-yl)-N,N,N′,N′-tetramethyluronium hexafluorophosphate (HATU), and O-(benzotriazol-1-yl)-N,N,N′,N′-tetramethyluronium tetrafluoroborate (TBTU). These reagents are commercially available and well-known to the skilled person.

Preferably, A^{x2} is selected from the group consisting of -maleimide, —SH, —NH₂, —SeH, —N₃, —C≡CH, —CR¹═CR^{1a}R^{1b}, —OH, —(CH═X)—R¹, —(C═O)—S—R¹, —(C═O)—H, —NH—NH₂, —O—NH₂, —Ar—X⁰, —Ar—Sn(R¹)(R^{1a})(R^{1b}), —Ar—B(OH)(OH), Br, I, Y¹—(C═O)—, Y¹—(C═O)—NH—, Y¹—(C═O)—O—,

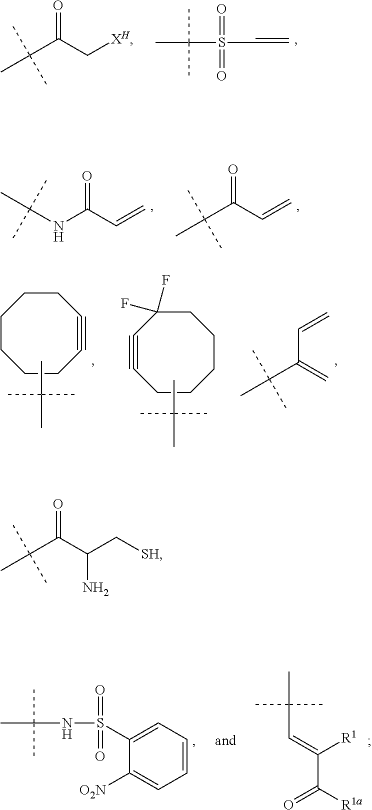

with optional protecting groups;
wherein
dashed lines indicate attachment to SP²;

X is O, S, or NH,
X¹ is —OH, —NR¹R^{1a}, —SH, or —SeH,
X^{H} is Cl, Br, I or F;

Ar is phenyl, naphthyl, indenyl, indanyl, or tetralinyl;

R¹, R^{1a}, R^{1b} are independently of each other H, C₁₋₆alkyl, C₂₋₆alkenyl, C₂₋₆alkynyl, C₃₋₈ cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi):

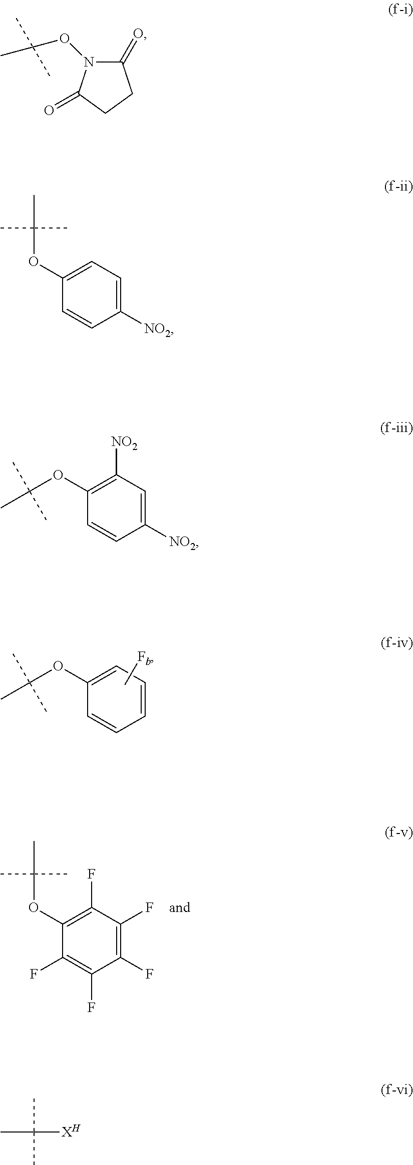

wherein
the dashed lines indicate attachment to the rest of the molecule,
b is 1, 2, 3 or 4,
X^{H} is Cl, Br, I, or F.

More preferably, A^{x2} is —NH₂, maleimide or thiol and most preferably A^{x2} is maleimide.

It is equally preferred that A^{x2} is thiol.

A^{x2} may optionally be present in protected form.

If the hydrogel of step (a) is covalently conjugated to a spacer moiety, the resulting hydrogel-spacer moiety conjugate is of formula (V):

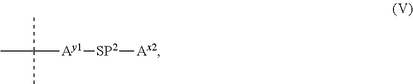

wherein
the dashed line indicates attachment to the hydrogel of step (a);
A^{y1} is the linkage formed between A^{x0} and A^{x1}; and
SP² and A^{x2} are used as in formula (I).

Preferably, A^{y1} is a stable linkage.

Preferably, A^{y1} is selected from the group consisting of

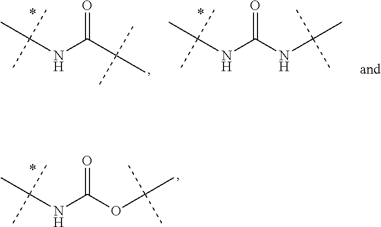

wherein
dashed lines marked with an asterisk indicate attachment to the hydrogel; and
unmarked dashed lines indicate attachment to SP².

Suitable reaction conditions are described in the Examples sections and are known to the person skilled in the art.

Process step (b) may be carried out in the presence of a base. Suitable bases include customary inorganic or organic bases. These preferably include alkaline earth metal or alkali metal hydrides, hydroxides, amides, alkoxides, acetates, carbonates or bicarbonates such as, for example, sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or ammonium carbonate, and tertiary amines such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN), N,N-diisopropylethylamine (DIPEA), diazabicycloundecene (DBU) or collidine.

Process step (b) may be carried out in the presence of a solvent. Suitable solvents for carrying out the process step (b) of the invention include organic solvents. These preferably include water and aliphatic, alicyclic or aromatic hydrocarbons such as, for example, petroleum ether, hexane, heptane, cyclohexane, methylcyclohexane, benzene, toluene, xylene or decalin; halogenated hydrocarbons such as, for example, chlorobenzene, dichlorobenzene, dichloromethane, chloroform, carbon tetrachloride, dichloroethane or trichloroethane; alcohols such as methanol, ethanol, n- or i-propanol, n-, i-, sec- or tert-butanol, ethanediol, propane-1,2-diol, ethoxyethanol, methoxyethanol, diethylene glycol monomethyl ether, dimethylether, diethylene glycol; acetonitrile, N-methyl-2-pyrrolidone (NMP), dimethylformamide (DMF), dimethyl sulfoxide (DMSO), N,N-dimethylacetamide, nitromethane, nitrobenzene, hexamethylphosphoramide (HMPT), 1,3-dimethyl-2-imidazolidinone (DMI), 1,3-dimethyl-3,4,5,6-tetrahydro-2(1H)-pyrimidinone (DMPU), ethyl acetate, acetone, butanone; ethers such as diethyl ether, diisopropyl ether, methyl t-butyl ether, methyl t-amyl ether, dioxane, tetrahydrofuran, 1,2-dimethoxyethane, 1,2-diethoxyethane or anisole; or mixtures thereof. Preferably, the solvent is selected from the group consisting of water, acetonitrile and N-methyl-2-pyrrolidone.

Step (c)

Preferably, A^{x3} is selected from the group consisting of —SH, —NH₂, —SeH, -maleimide, —C≡CH, —N₃, —CR¹═CR^{1a}R^{1b}, —(C═X)—R¹, —OH, —(C═O)—S—R¹, —NH—NH₂, —O—NH₂, —Ar—Sn(R¹)(R^{1a})(R^{1b}), —Ar—B(OH)(OH), —Ar—X⁰,

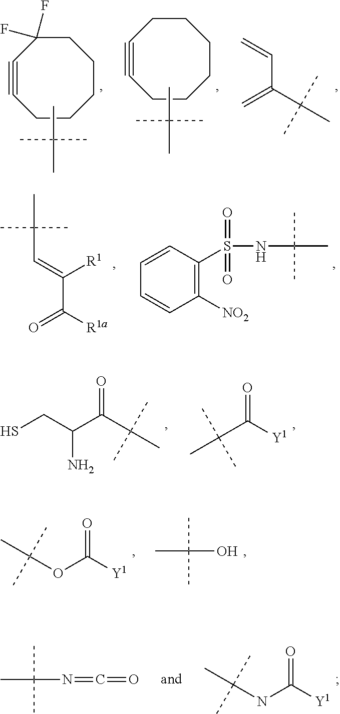

wherein
dashed lines indicate attachment to Z;
X is O, S, or NH,
X⁰ is —OH, —NR¹R^{1a}, —SH, or —SeH;
R¹, R^{1a}, R^{1b} are independently of each other H, C₁₋₆alkyl, C₂₋₆alkenyl, C₂₋₆alkynyl, C₃₋₈cycloalkyl, 4- to 7-membered heterocyclyl, 8- to 11-membered heterobicyclyl, phenyl, naphthyl, indenyl, indanyl, or tetralinyl; and
Ar is phenyl, naphthyl, indenyl, indanyl, or tetralinyl.
Y¹ is an activated carboxylic acid, activated carbonate or activated carbamate, preferably Y¹ is selected from formulas (f-i) to (f-vi):

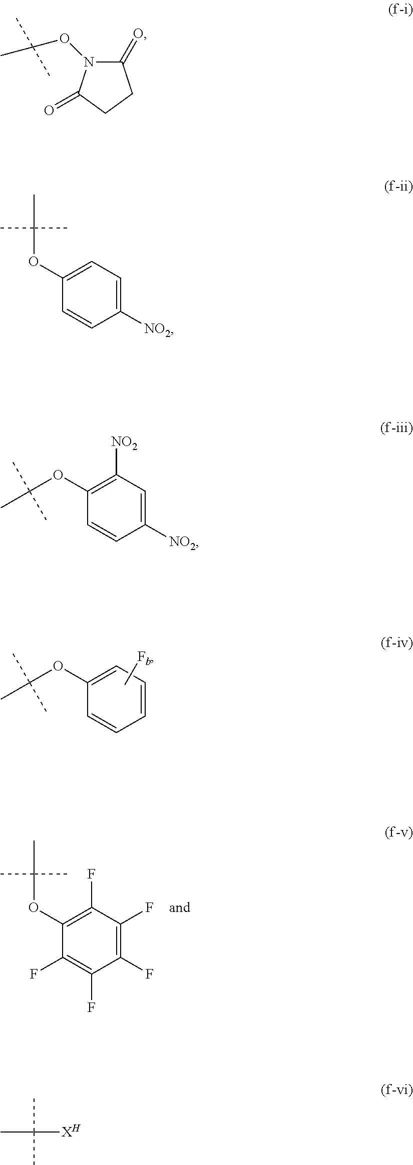

wherein
the dashed lines indicate attachment to the rest of the molecule,
b is 1, 2, 3 or 4,
X^{H} is Cl, Br, I, or F

In a preferred embodiment, Y¹ is selected from formulas (f-i) to (f-vi):

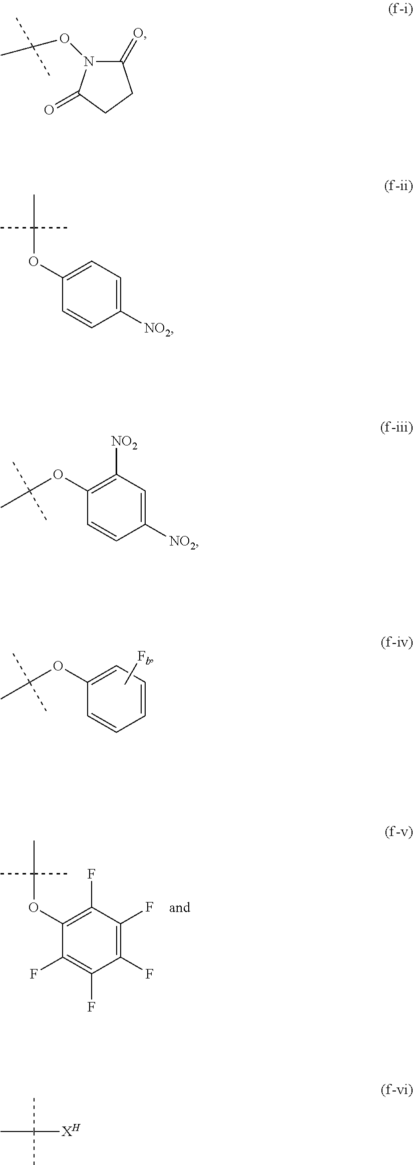

wherein
the dashed lines, b and X^{H} are used as above.

More preferably, A^{x3} is —SH or -maleimide and most preferably A^{x3} is —SH.

In another preferred embodiment A^{x3} is of formula (aI)

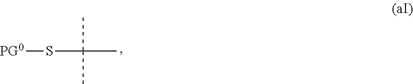

wherein
the dashed line indicates attachment to Z of formula (II);
PG⁰ is a sulfur-activating moiety; and
S is sulfur;

Preferably, PG⁰ of formula (aI) is selected from the group consisting of

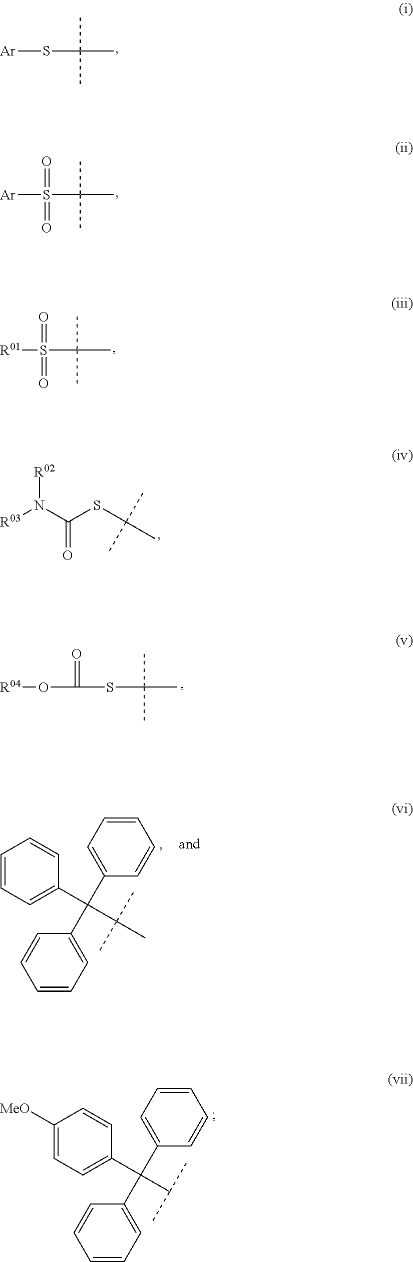

wherein
the dashed lines indicate attachment to the sulfur of formula (aI);
Ar is an aromatic moiety which is optionally further substituted;
R⁰¹, R⁰², R⁰³, R⁰⁴ are independently of each other —H; C₁₋₅₀ alkyl; C₂₋₅₀ alkenyl; or C₂₋₅₀ alkynyl, wherein C₁₋₅₀ alkyl; C₂₋₅₀ alkenyl; and C₂₋₅₀ alkynyl are optionally substituted with one or more R³, which are the same or different and wherein C₁₋₅₀ alkyl; C₂₋₅₀ alkenyl; and C₂₋₅₀ alkynyl are optionally interrupted by one or more groups selected from the group consisting of -Q-, —C(O)O—; —O—; —C(O)—; —C(O)N(R⁴)—; —S(O)₂N(R⁴)—; —S(O)N(R⁴)—; —S(O)₂—; —S(O)—; —N(R⁴)S(O)₂N(R^{4a})—; —S—; —N(R⁴)—; —OC(O)R⁴; —N(R⁴)C(O)—; —N(R⁴)S(O)₂—; —N(R⁴)S(O)—; —N(R⁴)C(O)O—; —N(R⁴)C(O)N(R^{4a})—; and —OC(O)N(R⁴R^{4a});
Q is selected from the group consisting of phenyl; naphthyl; indenyl; indanyl; tetralinyl; C₃₋₁₀ cycloalkyl; 4- to 7-membered heterocyclyl; and 8- to 11-membered heterobicyclyl, wherein T is optionally substituted with one or more R³, which are the same or different;
R³ is halogen; —CN; oxo (═O); —COOR⁵; —OR⁵; —C(O)R⁵; —C(O)N(R⁵R^{5a}); —S(O)₂N(R⁵R^{5a}); —S(O)N(R⁵R^{5a}); —S(O)₂R⁵; —S(O)R⁵; —N(R⁵)S(O)₂N(R^{5a}R^{5b}); —SR⁵; —N(R⁵R^{5a}); —NO₂; —OC(O)R⁵; —N(R⁵)C(O)R^{5a}; —N(R⁵)S(O)₂R^{5a}; —N(R⁵)S(O)R^{5a}; —N(R⁵)C(O)OR^{5a}; —N(R⁵)C(O)N(R^{5a}R^{5b}); —OC(O)N(R⁵R^{5a}); or C₁₋₆ alkyl, wherein C₁₋₆ alkyl is optionally substituted with one or more halogen, which are the same or different; and
   R⁴, R^{4a}, R⁵, R^{5a}, R^{5b} are independently selected from the group consisting of —H; or C₁₋₆alkyl, wherein C₁₋₆alkyl is optionally substituted with one or more halogen, which are the same or different.

Preferably, R⁰¹, R⁰³ and R⁰⁴ are independently of each other C₁₋₆alkyl.

Preferably, R⁰² is selected from H and C₁₋₆alkyl.

Preferably, Ar is selected from the group consisting of

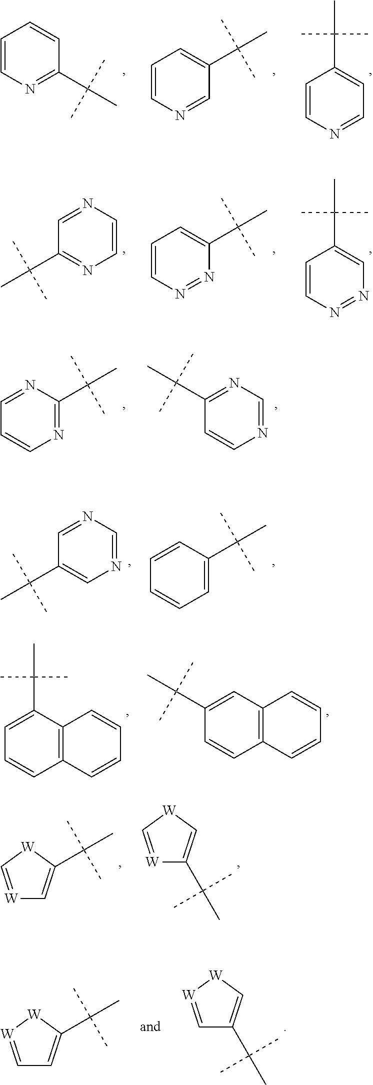

wherein

dashed lines indicate attachment to the rest of PG⁰ of formula (aI);

W is independently of each other O, S, or N;

W′ is N; and

wherein Ar is optionally substituted with one or more substituent(s) independently selected from the group consisting of NO₂, Cl and F.

More preferably, PG⁰ of formula (aI) is selected from the group consisting of

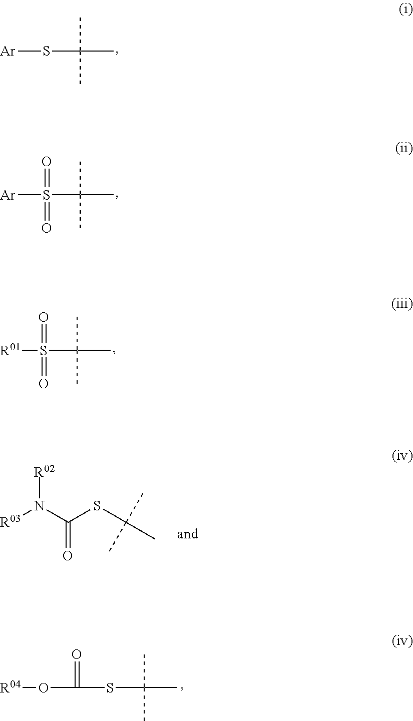

wherein
the dashed lines indicate attachment to the sulfur of formula (aI); and
Ar, R⁰¹, R⁰², R³ and R⁰⁴ are used as above.

More preferably, PG⁰ of formula (aI) is

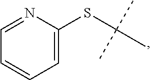

wherein
the dashed line indicates attachment to the sulfur of formula (aI).

A^{x3} may optionally be present in protected form.

Preferred combinations of A^{x2} and A^{x3} are the following:

| A^{x2} | A^{x3} |
|---|---|
| -maleimide | HS—, H₂N— or HSe— |
| -maleimide | —NH— |
| —SH, —NH₂ or —SeH | maleimide- |
| —NH— | maleimide- |
| —NH₂ | Y¹—(C═O)—, Y¹—(C═O)—NH—, |
| | or Y¹—(C═O)—O— |
| —N₃ | HC≡C—, |
| | 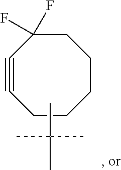 |
| | 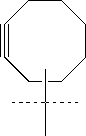 |
| —C≡CH, or | N₃— |
| 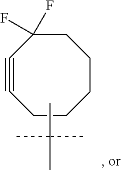 | |
| 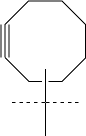 | |
| —CR^{1a}═CR^{la}R^{lb} | R^{1b}R^{1a}C═CR¹— or |
| | 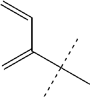 |
| 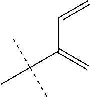 | R^{1b}R^{1a}C═CR¹— |
| —(C═X)—R¹ | 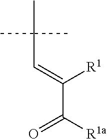 |
| 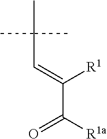 | R¹—(C═X)— |
| —OH | H₂N— or |
| | 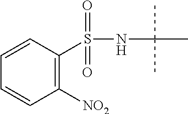 |
| —NH₂ or | HO— |
| 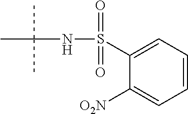 | |
| —(C═O)—S—R¹ | 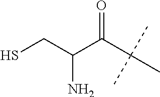 |
| 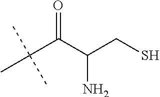 | R¹—S—(C═O)— |
| —(C═O)—H | H₂N—NH—or H₂N—O— |
| —NH—NH₂ or —O—NH₂ | H—(C═O)— |
| —Ar—X⁰ | —Ar—Sn(R¹)(R^{1a})(R^{1b}) or |
| | —Ar—B(OH)(OH) |
| (R^{1b})(R^{1a})(R¹)(Sn—Ar— or | X⁰—Ar— |
| —Ar—B(OH)(OH) | |

wherein
X is O, S, or NH;
X⁰ is —OH, —NR¹R^{1a}, —SH, or —SeH;

R¹, R^{1a}, R^{1b} are independently of each other selected from the group consisting of H, C₁₋₆alkyl, C₂₋₆alkenyl, C₂₋₆alkynyl, C₃₋₈cycloalkyl, 4- to 7-membered heterocyclyl, 8- to 11-membered heterobicyclyl, phenyl, naphthyl, indenyl, indanyl, and tetralinyl; and
Ar is phenyl, naphthyl, indenyl, indanyl, or tetralinyl.

In another preferred embodiment A^{x2} is —SH and A^{x3} is of formula (aI), wherein PG⁰ is of formula (i), (ii), (iii), (iv), (v), (vi) or (viii). More preferably, PG⁰ of formula (aI) is of formula (i), (ii), (iii), (iv) or (v) and even more preferably, PG⁰ of formula (aI) is of formula (i). Most preferably, PG⁰ of formula (aI) is of formula

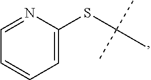

wherein
the dashed line indicates attachment to the sulfur of formula (aI).

In one preferred embodiment, A^{x2} is an amine and A^{x3} is Y¹—(C═O)—, Y¹—(C═O)—NH—, or Y¹—(C═O)—O— and most preferably A^{x2} is an amine and A^{x3} is Y¹—(C═O)—.

In another preferred embodiment A^{x2} is maleimide and A^{x3} is —SH.

In one embodiment the optional step (b) is omitted, A^{x0} is an amine and A^{x3} is ClSO₂—, R¹(C═O)—, I—, Br—, Cl—, SCN—, CN—, O═C═N—, Y¹—(C═O)—, Y¹—(C═O)—NH—, or Y¹—(C═O)—O—,
wherein
R¹ is H, C₁₋₆alkyl, C₂₋₆alkenyl, C₂₋₆alkynyl, C₃₋₈cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi):

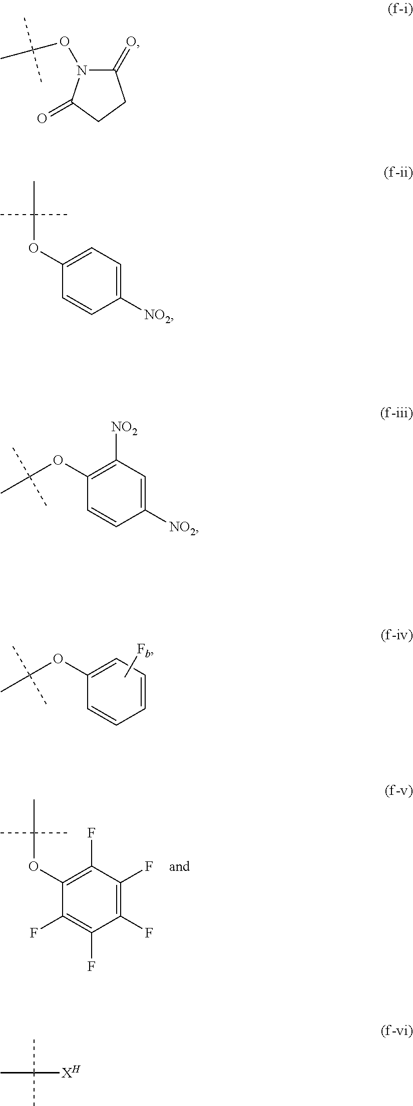

wherein
the dashed lines indicate attachment to the rest of the molecule,
b is 1, 2, 3 or 4,
X^{H} is Cl, Br, I, or F.

In another embodiment the optional step (b) is omitted, A^{x0} is a hydroxyl group (—OH) and A^{x3} is O═C═N—, I—, Br—, SCN—, or Y¹—(C═O)—NH—,
wherein Y¹ is selected from formulas (f-i) to (f-vi):

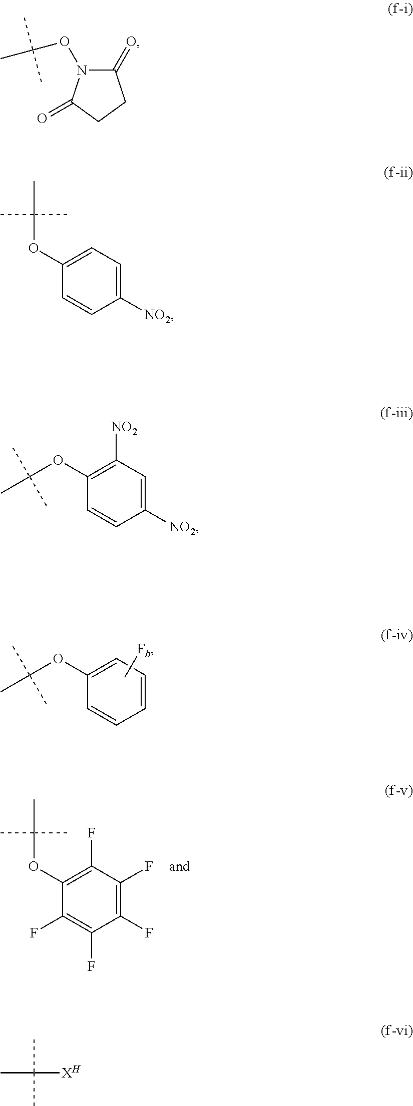

wherein
the dashed lines indicate attachment to the rest of the molecule,
b is 1, 2, 3 or 4,
X^{H} is Cl, Br, I, or F.

In another embodiment the optional step (b) is omitted, A^{x0} is a carboxylic acid (—(C═O)OH) and A^{x3} is a primary amine or secondary amine.

In another embodiment the optional step (b) is omitted, A^{x0} is an amine and A^{x3} is Y¹—(C═O)—, Y¹—(C═O)—NH—, or Y¹—(C═O)—O—.

In another embodiment the optional step (b) is omitted, A^{x0} is a maleimide and A^{x3} is thiol.

In a preferred embodiment the optional step (b) is omitted, A^{x0} is an amine and A^{x3} is Y—(C═O)—.

In another preferred embodiment the optional step (b) is omitted, A^{x0} is —SH and A^{x3} is of formula (aI), wherein PG⁰ is of formula (i), (ii), (iii), (iv), (v), (vi) or (viii). More preferably, PG⁰ of formula (aI) is of formula (i), (ii), (iii), (iv) or (v) and even more preferably, PG° of formula (aI) is of formula (i). Most preferably, PG⁰ of formula (aI) is of formula

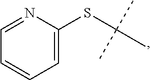

wherein
the dashed line indicates attachment to the sulfur of formula (aI).

The hydrogel obtained from step (c) has the structure of formula (VIa) or (VIb):

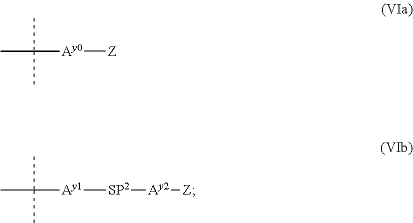

wherein
the dashed line indicates attachment to the hydrogel of step (a);
A^{y0} is the linkage formed between A^{x0} and A^{x3};
A^{y1} is used as in formula (V);
A^{y2} is the linkage formed between A^{x2} and A^{x3};
SP² is used as in formula (I); and
Z is used as in formula (II).

Preferably, A^{y0} and A^{y2} are selected from the group consisting of amide, carbamate,

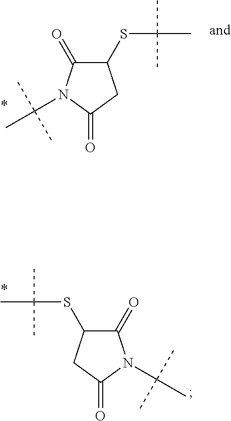

wherein
the dashed lines marked with an asterisk indicate attachment to the hydrogel or SP², respectively; and
the unmarked dashed lines indicate attachment to Z.

In one embodiment, Z is selected from the group consisting of C₁₋₅₀ alkyl, C₂₋₅₀ alkenyl, C₂₋₅₀ alkynyl, C₃₋₁₀ cycloalkyl, 4- to 7-membered heterocyclyl, 8- to 11-membered heterobicyclyl, phenyl; naphthyl; indenyl; indanyl; and tetralinyl; which C₁₋₅₀ alkyl, C₂₋₅₀ alkenyl, C₂₋₅₀ alkynyl, C₃₋₁₀ cycloalkyl, 4- to 7-membered heterocyclyl, 8- to 11-membered heterobicyclyl, phenyl; naphthyl; indenyl; indanyl; and tetralinyl are optionally substituted with one or more R¹⁰, which are the same or different and wherein C₁₋₅₀ alkyl; C₂₋₅₀ alkenyl; and C₂₋₅₀ alkynyl are optionally interrupted by one or more group(s) selected from the group consisting of T, —C(O)O—; —O—; —C(O)—; —C(O)N(R⁹)—; —S(O)₂N(R⁹)—; —S(O)N(R⁹)—; —S(O)₂—; —S(O)—; —N(R⁹)S(O)₂N(R^{9a})—; —S—; —N(R⁹)—; —OC(O)R⁹; —N(R⁹)C(O)—; —N(R⁹)S(O)₂—; —N(R⁹)S(O)—; —N(R⁹)C(O)O—; —N(R⁹)C(O)N(R^{9a})—; and —OC(O)N(R⁹R^{9a});
wherein
R⁹, R^{9a} are independently selected from the group consisting of H; T; C₁₋₅₀ alkyl; C₂₋₅₀ alkenyl; and C₂₋₅₀ alkynyl, which T; C₁₋₅₀ alkyl; C₂₋₅₀ alkenyl; and C₂₋₅₀ alkynyl are optionally substituted with one or more R¹⁰, which are the same or different and which C₁₋₅₀ alkyl; C₂₋₅₀ alkenyl; and C₂₋₅₀ alkynyl are optionally interrupted by one or more group(s) selected from the group consisting of T, —C(O)O—; —O—; —C(O)—; —C(O)N(R¹)—; —S(O)₂N(R¹)—; —S(O)N(R¹¹)—; —S(O)₂—; —S(O)—; —N(R¹¹)S(O)₂N(R^{1a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹R^{11a});
T is selected from the group consisting of phenyl; naphthyl; indenyl; indanyl; tetralinyl; C₃₋₁₀ cycloalkyl; 4- to 7-membered heterocyclyl; and 8- to 11-membered heterobicyclyl, wherein T is optionally substituted with one or more R¹⁰, which are the same or different;
R¹⁰ is halogen; CN; oxo (═O); COOR¹²; OR¹²; C(O)R¹²; C(O)N(R¹²R^{12a}); S(O)₂N(R¹²R^{12a}); S(O)N(R¹²R^{12a}); S(O)₂R¹²; S(O)R¹²; N(R¹²)S(O)₂N(R^{12a}R^{12b}); SR¹²; N(R¹²R^{12a}); NO₂; OC(O)R¹²; N(R¹²)C(O)R^{12a}; N(R¹²)S(O)₂R^{12a}; N(R¹²)S(O)R^{12a}; N(R¹²)C(O)OR^{12a}; N(R¹²)C(O)N(R^{12a}R^{12b}); OC(O)N(R¹²R^{12a}); or C₁₋₆alkyl, which C₁₋₆alkyl is optionally substituted with one or more halogen, which are the same or different;
R¹¹, R^{11a}, R¹², R^{12a}, R^{12b} are independently of each other selected from the group consisting of H; and C₁₋₆alkyl, which C₁₋₆alkyl is optionally substituted with one or more halogen, which are the same or different.

In another embodiment Z is an inert polymer having a molecular weight ranging from 0.5 kDa to 1000 kDa, preferably having a molecular weight ranging from 0.5 to 500 kDa, more preferably having a molecular weight ranging from 0.75 to 250 kDa, even more preferably ranging from 1 to 100 kDa, even more preferably ranging from 5 to 60 kDa, even more preferably from 10 to 50 kDa and most preferably Z has a molecular weight of 40 kDa.

Preferably, Z is an inert polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(imino carbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In a preferred embodiment Z is an inert linear or branched PEG-based polymer comprising at least 70% PEG or a hyaluronic acid-based polymer comprising at least 70% hyaluronic acid. More preferably, Z is an inert linear or branched PEG-based polymer comprising at least 70% PEG, even more preferably comprising at least 80% PEG and most preferably comprising at least 90% PEG.

In another preferred embodiment Z is a peptide or protein, which preferably has a molecular weight ranging from 0.5 to 100 kDa. More preferably, Z is a protein with a molecular weight ranging from 2 to 70 kDa, even more preferably, Z is a protein with a molecular weight ranging from 5 to 50 kDa.

In another preferred embodiment Z is a zwitterionic polymer. Preferrably, such zwitterionic polymer comprises poly(amino acids) and/or poly(acrylates).

As used herein, the terms “zwitterion” and “zwitterionic” refer to a neutral molecule or moiety with positive and negative charges at different locations within that molecule or moiety at the same time.

According to Zhang et al. (Nature Biotechnology, 2013, volume 31, number 6, pages 553-557) hydrogels made of zwitterionic polymers resist the foreign body response.

Step (c) comprises reacting the hydrogel of step (a) or step (b) with a reagent of formula (II) in such manner that no more than 99 mol-% of A^{x0} or A^{x2} react with A^{x3}. This can be achieved, for example, by reacting at most 0.99 chemical equivalents of the reagent of formula (II) relative to A^{x0} or A^{x2} with the hydrogel of step (a) or (b).

In order to prevent the reaction of more than 0.99 chemical equivalents, the reagent of formula (II) can be used in an amount of at most 0.99 chemical equivalents relative to A^{x0} or A^{x2} or, alternatively, the reaction rate is monitored and the reaction is interrupted when at most 0.99 chemical equivalents relative to A^{x0} or A^{x2} have reacted, especially when more than 0.99 chemical equivalents are used. It is understood that also due to physical constraints, such as steric hindrance, hydrophobic properties or other characteristics of the inert moiety Z, no more than 0.99 chemical equivalents may be capable of reacting with A^{x0} or A^{x2}, even if more chemical equivalents are added to the reaction.

Preferably, step (c) comprises reacting the hydrogel of step (a) or step (b) with a reagent of formula (II) in such manner that no more than 80 mol-% of A^{x0} or A^{x2} react with A^{x3}, even more preferably, such that no more than 60 mol-% of A^{x0} or A^{x2} react with A^{x3}, even more preferably, such that no more than 40 mol-% of A^{x0} or A^{x2} react with A^{x3}, even more preferably, such that no more than 20 mol-% of A^{x0} or A^{x2} react with A^{x3} and most preferably, such that no more than 15 mol-% of A^{x0} or A^{x2} react with A³.

This can be achieved, for example, by reacting at most 0.8, 0.6, 0.4, 0.2 or 0.15 chemical equivalents of the reagent of formula (II) relative to A^{x0} or A^{x2} with the hydrogel of step (a) or (b), respectively.

Methods to prevent the reaction of more chemical equivalents are described above.

Based on the measurements of the amount of substance of A^{x0} of step (a) and after step (c) the amount of substance of reacted A^{x0} can be calculated with equation (1):

Amount of substance of reacted A^{x0} in mmol/g=(A^{x0}₁−A^{x0}₂)/(A^{x0}₂×MW_{Z}+1), (1)
wherein
A^{x0}₁ is the amount of substance of functional groups A^{x0} of the hydrogel of step (a) in mmol/g;
A^{x0}₂ is the amount of substance of functional groups A^{x0} of the hydrogel after step (c) in mmol/g; and
MW_{Z} is the molecular weight of Z in g/mmol.

If the optional spacer reagent was covalently conjugated to the hydrogel of step (a), the calculation of the number of reacted A^{x2} is done accordingly.

The percentage of reacted functional groups A^{x0} relative to the functional groups A^{x0} of the hydrogel of step (a) is calculated according to equation (2):

mol-% of reacted A^{x0}=100×[(A^{x0}₁−A^{x0}₂)/(A^{x0}₂×MW_{Z}+1)]/A^{x0}₁, (2)
wherein the variables are used as above.

In one embodiment Z is conjugated to the surface of the hydrogel. This can be achieved by selecting the size and structure of the reagent A^{x3}-Z such that it is too large to enter the pores or network of the hydrogel. Accordingly, the minimal size of A^{x3}-Z depends on the properties of the hydrogel. The person skilled in the art however knows methods how to test whether a reagent A^{x3}-Z is capable of entering into the hydrogel using standard experimentation, for example by using size exclusion chromatography with the hydrogel as stationary phase.

Other Aspects

Another aspect of the present invention is a hydrogel obtainable from the process of the present invention.

Another aspect of the present invention is the use of the hydrogel of the present invention as a carrier in a hydrogel-linked prodrug.

Another aspect of the present invention is a hydrogel-linked prodrug comprising a covalently conjugated hydrogel of the present invention.

It is preferred that the conjugates of formula (VIa) and (VIb) are such that they shield the biologically active moieties conjugated to the surface of carrier-linked prodrugs in which the hydrogels of the present invention are used as carriers.

This can be tested by using immune assays in which labelled antibodies against the biologically active moiety are used to test the binding of said labelled antibodies to biologically active moieties conjugated to hydrogels of step c) and of step a) and to calculate the relative binding of the labelled antibodies to biologically active moieties conjugated to hydrogels of step c) relative to those conjugated to hydrogels of step a).

Preferably, the binding of such labelled antibody to a biologically active moiety conjugated to a hydrogel of step c) is no more than 50% of the binding of said labelled antibody to said biologically active moiety conjugated to a hydrogel of step a), e.g. no more than 40%, no more than 30%, no more than 20%, no more than 10%, no more than 5%, no more than 2% or no more than 1%.

It was surprisingly found that hydrogels of the present invention that carry polymeric moieties Z effectively shield remaining functional groups on the surface of the hydrogel and/or effectively shield biologically active moieties conjugated to the surface of the hydrogel.

When such hydrogel is used as a carrier in a carrier-linked prodrug it has the technical effect of rendering the carrier-linked prodrug better tolerable by the patient and causes reduced immune responses.

Another technical effect obtained with the hydrogels of the present invention is that carrier-linked prodrugs comprising such hydrogels can be injected using reduced force, in particular in the case of carrier-linked prodrugs of hydrophobic drugs.

In another embodiment the process of the present invention can be performed such that instead of the hydrogel a hydrogel-linked prodrug is used in step (a) and steps (b) and (c) are performed as detailed above.

### EXAMPLES

### Materials and Methods

Materials:

Amino 4-arm PEG5000 was obtained from JenKem Technology, Beijing, P. R. China. Cithrol™ DPHS was obtained from Croda International Pic, Cowick Hall, United Kingdom.

Isopropylmalonic acid was obtained from ABCR GmbH & Co. KG, 76187 Karlsruhe, Germany.

N-maleimido propionic acid NHS-ester was obtained from TCI Deutschland, 65760 Eschborn, Germany.

All other chemicals were from Sigma-ALDRICH Chemie GmbH, Taufkirchen, Germany.

5 kDa PEG-SH, 10 kDa PEG-SH, 20 kDa PEG-SH, 10 kDa PEG-NHS and 20 kDa PEG-NHS were obtained from RAPP POLYMERE GmbH, 72072 Tiibingen, Germany.

N-(3-maleimidopropionyl)-21-amino-4,7,10,13,16,19-hexaoxa-heneicosanoic acid Pfp ester (Mal-PEG₆-Pfp) was obtained from Biomatrik, China.

30 kDa PEG-NHS, 40 kDa PEG-NHS, branched 40 kDa PEG-NHS, branched 60 kDa PEG-NHS and branched 80 kDa PEG-NHS were obtained from NOF Corporation, Tokyo 150-6019, Japan.

Methods:

RP-HPLC was done on a 100×20 mm or 100×40 mm C18 ReproSil-Pur 300 ODS-3 5μ column (Dr. Maisch, Ammerbuch, Germany) connected to a Waters 600 or 2535 HPLC System and Waters 2487 or 2489 Absorbance detector, respectively. Linear gradients of solution A (0.1% TFA in H2O) and solution B (0.1% TFA in acetonitrile) were used. HPLC fractions containing product were combined and lyophilized.

Flash chromatography purifications were performed on an Isolera One system from Biotage AB, Sweden, using Biotage KP-Sil silica cartridges and n-heptane, ethyl acetate, and methanol as eluents. Products were detected at 254 nm. For products showing no absorbance above 240 nm fractions were screened by LC/MS.

Analytical ultra-performance LC (UPLC) was performed on a Waters Acquity system equipped with a Waters BEH300 C18 column (2.1×50 mm, 1.7 μm particle size) coupled to a LTQ Orbitrap Discovery mass spectrometer from Thermo Scientific.

HPLC-Electrospray ionization mass spectrometry (HPLC-ESI-MS) was performed on a Waters Acquity UPLC with an Acquity PDA detector coupled to a Thermo LTQ Orbitrap Discovery high resolution/high accuracy mass spectrometer equipped with a Waters ACQUITY UPLC BEH300 C18 RP column (2.1×50 mm, 300 Å, 1.7 μm, flow: 0.25 mL/min; solvent A: UP-H₂O+0.04% TFA, solvent B: UP-Acetonitrile+0.05% TFA.

MS spectra of PEG products showed a series of (CH₂CH₂O)ₙ moieties due to polydispersity of PEG staring materials. For easier interpretation only one single representative m/z signal is given in the examples.

### Example 1

### Synthesis of Backbone Reagent 1a and 1b

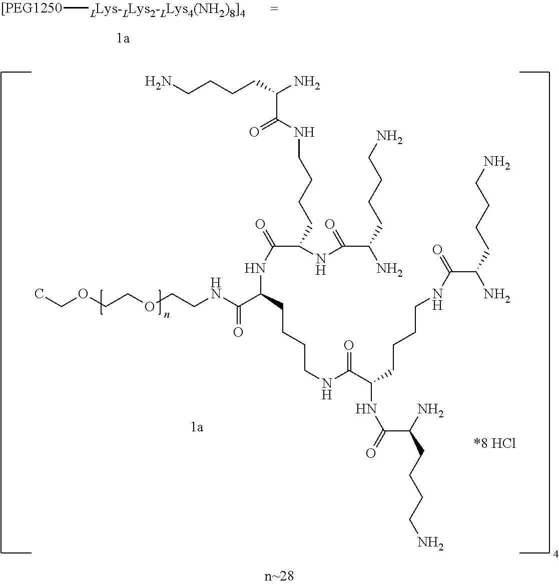

Backbone reagent 1a was synthesized as described in example 1 of WO 2011/012715 A1. Backbone reagent 1b was synthesized as described in example 1 of WO 2011/012715 A1 except for the use of Boc-DLys(Boc)-OH instead of Boc-LLys(Boc)-OH.

MS: m/z 888.50=[M+10H⁺]¹⁰⁺ (calculated=888.54)

For mass spectra of polydisperse PEG containing compounds, one single mass peak was selected.

### Example 2

### Synthesis of Crosslinker Reagents 2d, Rac-2h, 2k, 2n, Rac-2q, and Rac-2t

Crosslinker reagent 2d was prepared from azelaic acid monobenzyl ester and PEG2000 according to the following scheme:

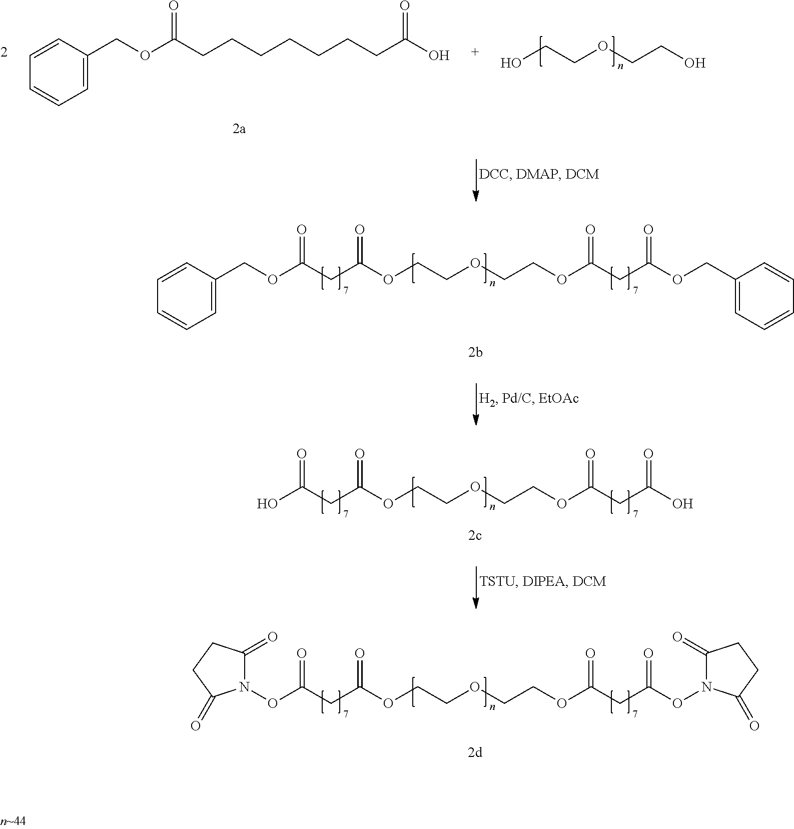

For the synthesis of azelaic acid monobenzyl ester 2a, a mixture of azelaic acid (37.6 g, 200 mmol), benzyl alcohol (21.6 g, 200 mmol), p-toluenesulfonic acid (0.80 g, 4.2 mmol), and 240 mL toluene was heated to reflux for 7 h in a Dean-Stark apparatus. After cooling down, the solvent was evaporated and 300 mL sat. aqueous NaHCO₃ solution were added. This mixture was extracted with 3×200 mL MTBE. The combined organic phases were dried over Na₂SO₄ and the solvent was evaporated. The product was purified on 2×340 g silica using ethyl acetate/heptane (10:90-25:75) as eluent. The eluent was evaporated and the residue was dried in vacuo over night.

Yield 25.8 g (46%) colorless oil 2a.

MS: m/z 279.16=[M+H]⁺ (calculated=279.16).

For synthesis of compound 2b, azelaic acid monobenzyl ester 2a (14.6 g, 52.5 mmol) and PEG 2000 (30.0 g, 15 mmol) were dissolved in 50 mL dichloromethane and cooled with an ice bath. A solution of DCC (10.8 g, 52.5 mmol) and DMAP (91.6 mg, 0.8 mmol) in 30 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 26 mL dichloromethane and diluted with 780 mL MTBE at room temperature. The mixture was stored over night at −20° C. The precipitate was collected by filtration through a glass filter Por. 3 and washed with 350 mL of cooled MTBE (−20° C.).

The product was dried in vacuo over night.

Yield 32.5 g (86%) white powder 2b.

MS: m/z=826.49 [M+3H]³⁺ (calculated=856.05).

For synthesis of compound 2c, compound 2b (32.2 g, 12.8 mmol) was dissolved in ethyl acetate (196 mL) and 306 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 28.8 g (96%) glassy solid 2c.

MS: m/z 751.78=[M+3H]³⁺ (calculated=751.91).

For mass spectra of polydisperse PEG containing compounds, one single mass peak was selected.

For synthesis of compound 2d, compound 2c (28.7 g, 12.3 mmol) and TSTU (14.8 g, 49.1 mmol) were dissolved in 100 mL dichloromethane at room temperature. Then DIPEA (6.3 g, 49.1 mmol) was added and the mixture was stirred for 1 h. The resulting suspension was filtered and 170 mL dichloromethane was added. The filtrate was washed with 200 mL aqueous solution (3 g NaOH, 197 g NaCl and 750 g H₂O). The organic phase was dried over MgSO₄ and the solvent was evaporated in vacuo. The residue was dissolved in 200 mL toluene, diluted with 200 mL MTBE at room temperature and stored over night at −20° C. The precipitate was collected by filtration through a glass filter Por. 3 and washed with 250 mL of cooled MTBE (−20° C.). The product was dried in vacuo over night.

Yield 24.0 g (77.4%) white powder 2d.

MS: m/z 845.82=[M+3H]³⁺ (calculated=860.68).

For mass spectra of polydisperse PEG containing compounds, one single mass peak was selected.

Synthesis of Crosslinker Reagent Rac-2h

Crosslinker reagent rac-2h was prepared from isopropylmalonic acid monobenzyl ester and PEG3300 according to the following scheme:

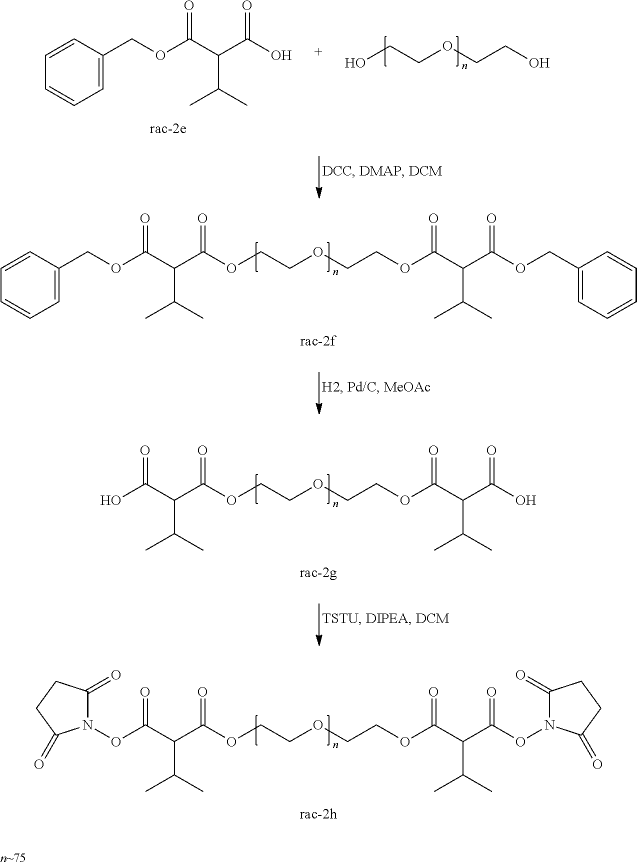

For the synthesis of isopropylmalonic acid monobenzyl ester rac-2e, isopropylmalonic acid (35.0 g, 239 mmol), benzyl alcohol (23.3 g, 216 mmol) and DMAP (1.46 g, 12.0 mmol) were dissolved in 100 mL acetonitrile. Mixture was cooled to 0° C. with an ice bath. A solution of DCC (49.4 g, 239 mmol) in 150 mL acetonitrile was added within 15 min at 0° C. The ice bath was removed and the reaction mixture was stirred over night at room temperature, then the solid was filtered off. The filtrate was evaporated at 40° C. in vacuo and the residue was dissolved in 300 mL MTBE. This solution was extracted with 2×300 mL sat. aqueous NaHCO₃ solution, then the combined aqueous phases were acidified to pH=1-3 using 6 N hydrochloric acid. The resulting emulsion was extracted with 2×300 mL MTBE and the solvent was evaporated. The combined organic phases were washed with 200 mL sat. aqueous NaCl and dried over MgSO₄. The product was purified on 340 g silica using ethyl acetate/heptane (10:90→20:80) as eluent. The eluent was evaporated and the residue was dried in vacuo over night.

Yield 9.62 g (17%) colorless oil rac-2e.

MS: m/z 237.11=[M+H]⁺ (calculated=237.11).

For synthesis of compound rac-2f, isopropylmalonic acid monobenzyl ester rac-2e (5.73 g, 24.2 mmol) and PEG 3300 (20.0 g, 6.06 mmol) were dissolved in 105 mL dichloromethane and cooled with an ice bath. A solution of DCC (5.00 g, 24.2 mmol) and DMAP (37 mg, 0.30 mmol) in 15 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 70 mL dichloromethane and diluted with 725 mL MTBE at room temperature. The mixture was stored over night at −20° C. The precipitate was collected by filtration through a glass filter Por. 3 and washed with 650 mL of cooled MTBE (−20° C.). The product was dried in vacuo over night.

Yield 21.3 g (94%) white powder rac-2f.

MS: m/z 671.39=[M+6H]⁶⁺ (calculated=671.47).

For mass spectra of polydisperse PEG containing compounds, one single mass peak was selected.

For synthesis of compound rac-2g, compound rac-2f (21.2 g, 5.65 mmol) was dissolved in ethyl acetate (130 mL) and 234 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 20.0 g (99%) glassy solid rac-2g.

MS: m/z 597.35=[M+6H]⁶⁺ (calculated=597.38).

For mass spectra of polydisperse PEG containing compounds, one single mass peak was selected.

For synthesis of compound rac-2h, compound rac-2g (7.50 g, 2.10 mmol) and TSTU (2.52 g, 8.38 mmol) were dissolved in 105 mL dichloromethane at room temperature. Then DIPEA (1.08 g, 8.38 mmol) was added and the mixture was stirred for 45 min. The reaction mixture was filtered into 8 individual 50 mL PP Falcon tubes, then 10 mL phosphate buffer 0.5 M pH=6.5 was added into each Falcon tubes. The mixture was shaken and centrifuged (2000 min⁻¹, 1 min). The lower (organic) phases were removed, combined and centrifuged (2000 min⁻¹, 1 min) again. Then the lower phases were dried over MgSO₄ and the solvent was evaporated in vacuo. The residue was dissolved in 100 mL toluene, filtered, diluted with 145 mL MTBE at room temperature and stored over night at −20° C. The precipitate was collected by filtration through a glass filter Por. 3 and washed with 500 mL of cooled MTBE (−20° C.). The product was dried in vacuo over night.

Yield 7.19 g (91%) white powder rac-2h.

MS: m/z 673.71=[M+6H]⁶⁺ (calculated=673.77).

For mass spectra of polydisperse PEG containing compounds, one single mass peak was selected.

Crosslinker reagent 2k was prepared from azelaic acid monobenzyl ester and PEG4000 according to the following scheme:

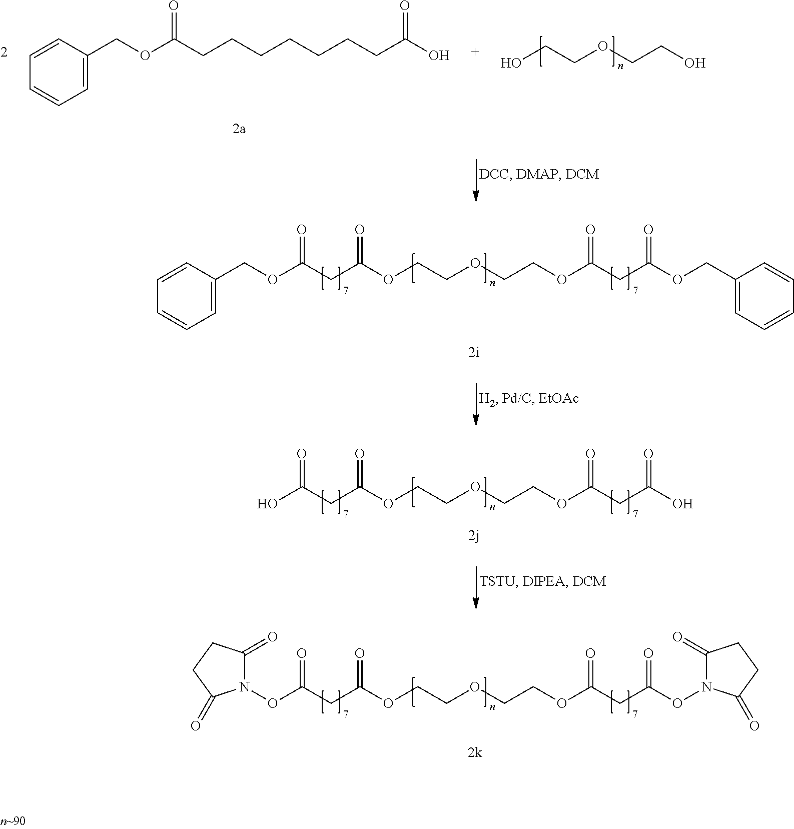

For synthesis of compound 2i, azelaic acid monobenzyl ester 2a (9.74 g, 35.0 mmol) and PEG 4000 (40.0 g, 10.0 mmol) were dissolved in 130 mL dichloromethane and cooled with an ice bath. A solution of DCC (7.22 g, 35.0 mmol) and DMAP (61 mg, 0.5 mmol) in 40 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 59 mL dichloromethane and diluted with 314 mL MTBE at room temperature. The mixture was stored over night at −20° C. The precipitate was collected by filtration through a glass filter Por. 3 and washed with 250 mL of cooled MTBE (−20° C.). The product was dried in vacuo over night.

Yield 42.9 g (95%) white powder 2i.

MS: m/z 795.48=[M+6H]⁶⁺ (calculated=751.58).

For mass spectra of polydisperse PEG containing compounds, one single mass peak was selected.

For synthesis of compound 2j, compound 2i (42.8 g, 9.47 mmol) was dissolved in ethyl acetate (230 mL) and 35 mL ethanol and 400 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 37.5 g (91%) glassy solid 2j.

MS: m/z 758.13=[M+6H]⁶⁺ (calculated=721.54).

For mass spectra of polydisperse PEG containing compounds, one single mass peak was selected.

For synthesis of compound 2k, compound 2j (37.3 g, 8.60 mmol) and TSTU (10.4 g, 34.4 mmol) were dissolved in 120 mL dichloromethane at room temperature. Then DIPEA (4.45 g, 34.4 mmol) was added and the mixture was stirred for 45 min. The resulting suspension was filtered and 100 mL dichloromethane was added. The filtrate was washed with 200 mL aqueous solution (3 g NaOH, 197 g NaCl and 750 g H₂O). The organic phase was dried over MgSO₄ and the solvent was evaporated in vacuo. The residue was dissolved in 200 mL toluene, diluted with 380 mL MTBE at room temperature and stored over night at −20° C. The precipitate was collected by filtration through a glass filter Por. 3 and washed with 450 mL of cooled MTBE (−20° C.). The product was dried in vacuo over night.

Yield 32.7 g (84%) white powder 2k.

MS: m/z 790.46=[M+6H]⁶⁺ (calculated=753.90).

For mass spectra of polydisperse PEG containing compounds, one single mass peak was selected.

Crosslinker reagent 2n was prepared from suberic acid monobenzyl ester 2a and PEG6000 accordingly to the following scheme:

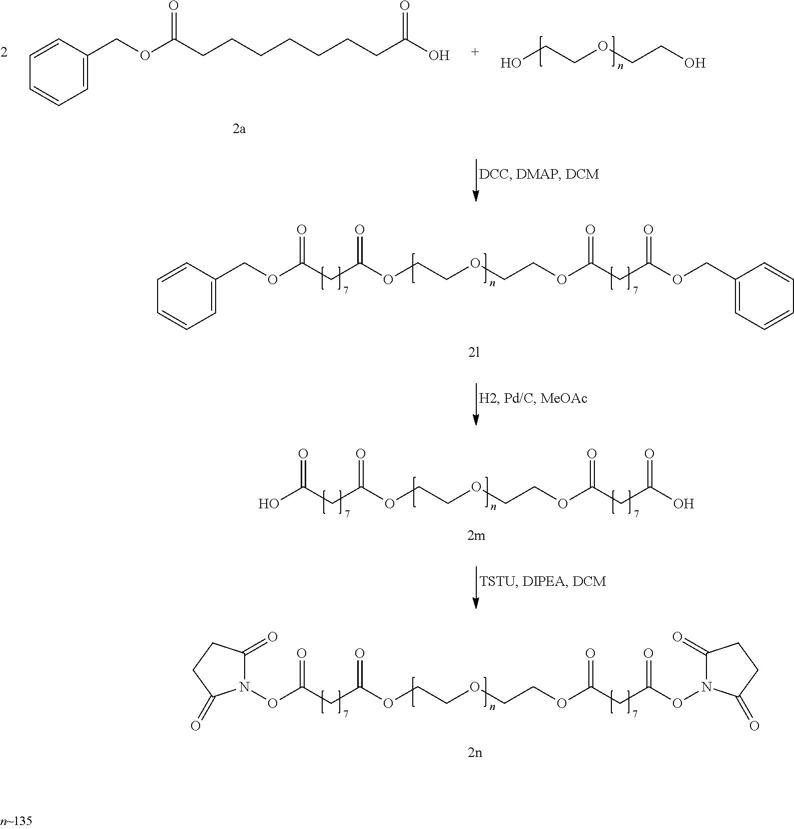

For synthesis of compound 21, azelaic acid monobenzyl ester 2a (6.50 g, 23.3 mmol) and PEG 6000 (40.0 g, 6.67 mmol) were dissolved in 140 mL dichloromethane and cooled with an ice bath. A solution of DCC (4.81 g, 23.3 mmol) and DMAP (0.040 g, 0.33 mmol) in 40 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 70 mL dichloromethane and diluted with 300 mL MTBE at room temperature. The mixture was stored over night at −20° C. The precipitate was collected by filtration through a glass filter Por. 3, and washed with 500 mL of cooled MTBE (−20° C.).

The product was dried in vacuo over night.

Yield 41.2 g (95%) white powder 21.

MS: m/z 833.75=[M+8H]⁸⁺ (calculated=833.74).

For mass spectra of polydisperse PEG containing compounds, one single mass peak was selected.

For synthesis of compound 2m, compound 21 (41.2 g, 6.32 mmol) was dissolved in methyl acetate (238 mL) and ethanol (40 mL), then 400 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 38.4 g (96%) glassy solid 2m.

MS: m/z 750.46=[M+9H]⁹⁺ (calculated=750.56).

For mass spectra of polydisperse PEG containing compounds, one single mass peak was selected.

For synthesis of compound 2n, compound 2m (38.2 g, 6.02 mmol) and TSTU (7.25 g, mmol) were dissolved in 130 mL dichloromethane at room temperature. Then DIPEA (3.11 g, 24.1 mmol) was added and the mixture was stirred for 1 h. The resulting suspension was filtered, the filtrate was diluted with 100 mL dichloromethane and washed with 200 mL of a solution of 750 g water/197 g NaCl/3 g NaOH. The organic phase was dried over MgSO₄ and the solvent was evaporated in vacuo.

The residue was dissolved in 210 mL toluene, diluted with 430 mL MTBE at room temperature and stored over night at −20° C. The precipitate was collected by filtration through a glass filter Por. 3, and washed with 450 mL of cooled MTBE (−20° C.). The product was dried in vacuo over night.

Yield 35.8 g (91%) white powder 2n.

MS: m/z 857.51=[M+8H]⁸⁺ (calculated=857.51).

For mass spectra of polydisperse PEG containing compounds, one single mass peak was selected.

Crosslinker reagent 2q was prepared from isopropylmalonic acid monobenzyl ester and PEG8000 according to the following scheme:

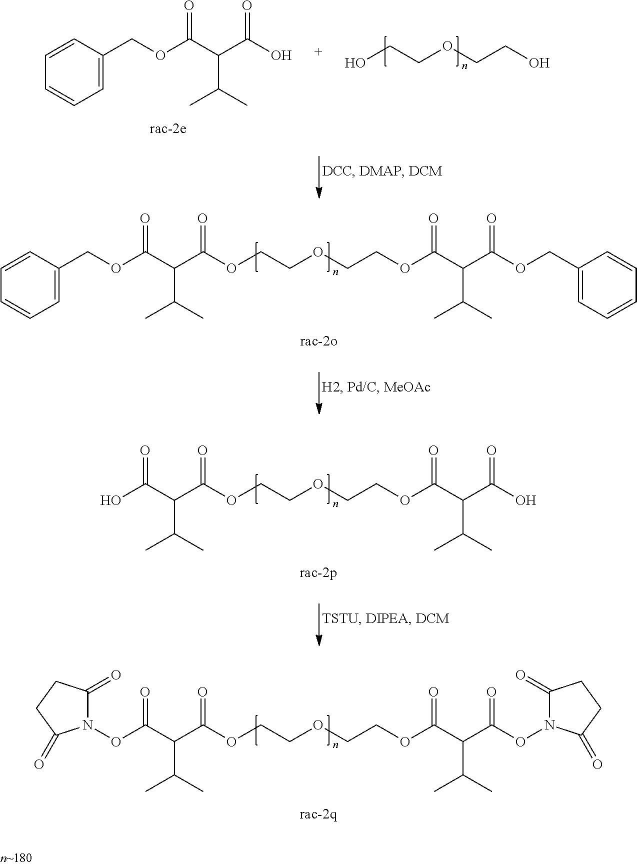

For synthesis of compound rac-2o, isopropylmalonic acid monobenzyl ester rac-2e (2.25 g, 9.50 mmol) and PEG 8000 (19.0 g, 2.38 mmol) were dissolved in 100 mL dichloromethane and cooled with an ice bath. A solution of DCC (1.96 g, 9.50 mmol) and DMAP (14 mg, 0.12 mmol) in 10 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 40 mL dichloromethane and diluted with 270 mL MTBE at room temperature. The mixture was stored over night at −20° C. The precipitate was collected by filtration through a glass filter Por. 3, and washed with 500 mL of cooled MTBE (−20° C.). The product was dried in vacuo over night.

Yield 18.5 g (92%) white powder rac-2o.

MS: m/z 737.43=[M+13H]¹³⁺ (calculated=737.42).

For mass spectra of polydisperse PEG containing compounds, one single mass peak was selected.

For synthesis of compound rac-2p, compound rac-2o (18.4 g, 2.18 mmol) was dissolved in methyl acetate (160 mL) and 254 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 17.7 g (98%) glassy solid rac-2p.

MS: m/z 723.51=[M+13H]¹³⁺ (calculated=723.55).

For mass spectra of polydisperse PEG containing compounds, one single mass peak was selected.

For synthesis of compound rac-2q, compound rac-2p (13.6 g, 1.65 mmol) and TSTU (1.96 g, 6.60 mmol) were dissolved in 60 mL dichloromethane at room temperature. Then DIPEA (852 mg, 6.60 mmol) was added and the mixture was stirred for 45 min. The resulting suspension was filtered, the filtrate was diluted with 70 mL ethyl acetate and washed with 70 mL of a 0.5 M phosphate buffer pH=6.5. The organic phase was dried over MgSO₄ and the solvent was evaporated in vacuo. The residue was dissolved in 80 mL toluene, the remaining solid was filtered off and washed with 20 mL of toluene. The combined toluene fractions were diluted with 35 mL MTBE at room temperature and stored over night at −20° C. The precipitate was collected by filtration through a glass filter Por. 3, and washed with 600 mL of cooled MTBE (−20° C.). The product was dried in vacuo over night.

Yield 12.1 g (87%) white powder rac-2q.

MS: m/z 738.51=[M+13H]¹³⁺ (calculated=738.49).

For mass spectra of polydisperse PEG containing compounds, one single mass peak was selected.

Crosslinker reagent 2t was prepared from isopropylmalonic acid monobenzyl ester and PEG10000 according to the following scheme:

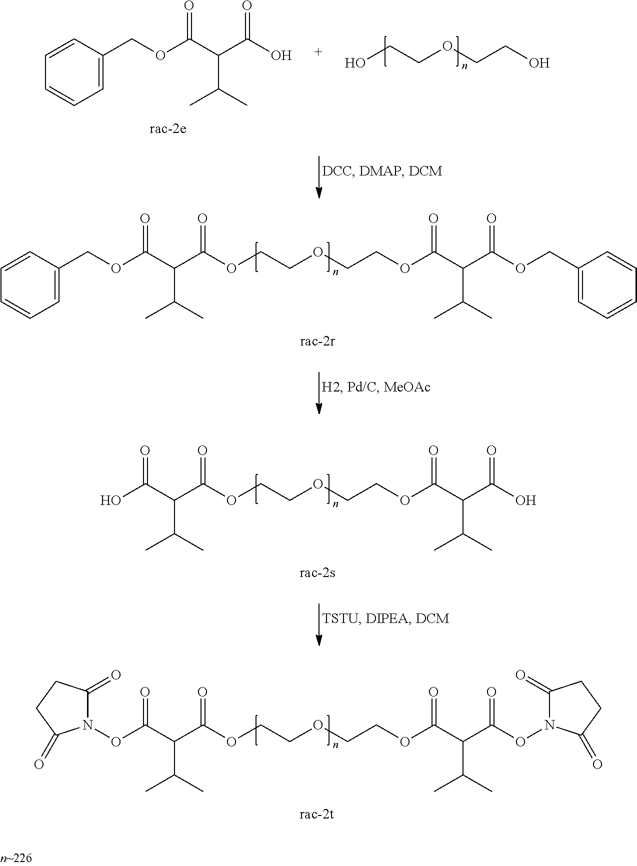

For synthesis of compound rac-2r, isopropylmalonic acid monobenzyl ester rac-2e (945 mg, 4.00 mmol) and PEG 10000 (10.0 g, 4.00 mmol) were dissolved in 20 mL dichloromethane and cooled with an ice bath. A solution of DCC (825 mg, 4.00 mmol) and DMAP (6 mg, 0.05 mmol) in 10 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 20 mL dichloromethane and diluted with 150 mL MTBE at room temperature. The mixture was stored over night at −20° C. The precipitate was collected by filtration through a glass filter Por. 3, and washed with 500 mL of cooled MTBE (−20° C.). The product was dried in vacuo over night.

Yield 9.63 g (92%) white powder rac-2r.

MS: m/z 742.50=[M+16H]¹⁶⁺ (calculated=742.51).

For mass spectra of polydisperse PEG containing compounds, one single mass peak was selected.

For synthesis of compound rac-2s, compound rac-2r (3.38 g, 0.323 mmol) was dissolved in methyl acetate (100 mL) and 105 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 3.25 g (98%) glassy solid rac-2s.

MS: m/z 731.25=[M+16H]¹⁶⁺ (calculated=731.25).

For mass spectra of polydisperse PEG containing compounds, one single mass peak was selected.

For synthesis of compound rac-2t, compound rac-2s (3.10 g, 0.302 mmol) and TSTU (0.364 g, 1.21 mmol) were dissolved in 15 mL dichloromethane at room temperature. Then DIPEA (0.156 g, 1.21 mmol) was added and the mixture was stirred for 45 min. The resulting suspension was filtered and the filtrate was washed with 2×10 mL of a 0.5 M phosphate buffer pH=6.5. The organic phase was dried over MgSO₄ and the solvent was evaporated in vacuo. The residue was dissolved in 20 mL toluene, diluted with 10 mL MTBE at room temperature and stored over night at −20° C. The precipitate was collected by filtration through a glass filter Por. 3, and washed with 250 mL of cooled MTBE (−20° C.). The product was dried in vacuo over night.

Yield 2.66 g (84%) white powder rac-2t.

MS: m/z 743.37=[M+16H]¹⁶⁺ (calculated=743.38).

For mass spectra of polydisperse PEG containing compounds, one single mass peak was selected.

### Example 3

### Preparation of Hydrogel Beads 3a Containing Free Amino Groups

In a cylindrical 250 mL reactor with bottom outlet, diameter 60 mm, equipped with baffles, an emulsion of 258 mg Cithrol™ DPHS in 100 mL undecane was stirred with an isojet stirrer, diameter 50 mm at 750 rpm, at ambient temperature. A solution of 2400 mg 1a and 3120 mg 2d in 22.1 g DMSO was added and stirred for 10 min to form a suspension. 10.7 mL TMEDA were added to effect polymerization. The mixture was stirred for 16 h at ambient temperature. 16.5 mL of acetic acid were added and then after 10 min 100 mL of a 15 wt % solution of sodium chloride in water was added. After 10 min, the stirrer was stopped and phases were allowed to separate. After 2 h the aqueous phase containing the hydrogel was drained.

For bead size fractionation, the water-hydrogel suspension was diluted with 50 mL ethanol and wet-sieved on 100, 75, 63, 50, 40, and 32 μm steel sieves using a Retsch AS200 control sieving machine for 15 min. Sieving amplitude was 1.5 mm, eluent was 3 L of 15 wt % aqueous NaCl solution, then 1 L of pure water, both with a flow of 300 mL/min. Bead fractions that were retained on the 40, 50, 63, and 75 μm sieves were washed 3 times with 0.1% AcOH, 10 times with ethanol and dried for 16 h at 0.1 mbar to give 0.65 g, 0.82 g, 0.42 g, and 0.07 g respectively, of 3a as a white powder.

Amino group content of the hydrogel was of the 75 μm fraction was determined to be 1.003 mmol/g by conjugation of a Fmoc-amino acid to the free amino groups on the hydrogel and subsequent Fmoc-determination as described by Gude, M., J. Ryf, et al. (2002) Letters in Peptide Science 9(4): 203-206.

Preparation of Hydrogel Beads 3b Containing Free Amino Groups.

3b was prepared as described for 3a except applying a stirrer speed of 740 rpm, the use of 2570 mg 1b, 3341 mg 2d, 23.6 g DMSO, 257 mg Cithrol™ DPHS, 11.5 mL TMEDA, 17.6 mL acetic acid, yielding 0.20 g on the 40 μm sieve, 0.37 g on the 50 μm sieve, 0.81 g on the 63 μm sieve, and 0.68 g on the 75 μm sieve of 3b as a white powder, free amino groups 1.020 mmol/g.

Preparation of Hydrogel Beads 3c Containing Free Amino Groups.

3c was prepared as described for 3a except applying a stirrer speed of 480 rpm, the use of 1000 mg 1b, 4466 mg rac-2h, 49.2 g DMSO, 486 mg Cithrol™ DPHS, 4.5 mL TMEDA, 6.9 mL acetic acid, sieving on 125, 100, 75, 63, 50, 40, and 32 μm steel sieves using 4 L of pure water as an eluent, yielding 0.14 g on the 40 μm sieve, 0.20 g on the 50 μm sieve, 0.26 g on the 63 μm sieve, 1.17 g on the 75 μm sieve, and 0.50 g on the 100 μm sieve of 3c as a white powder, free amino groups 0.201 mmol/g.

Preparation of Hydrogel Beads 3d Containing Free Amino Groups.

3d was prepared as described for 3c except using a 1000 mL reactor with 100 mm diameter, applying a stirrer speed of 520 rpm, the use of 565 mg Cithrol™ DPHS in 440 mL undecane, 1000 mg 1b, 5355 mg 2k, 57.2 g DMSO, 4.5 mL TMEDA, 6.9 mL acetic acid, addition of 200 mL 15 wt % solution of sodium chloride in water and after phase separation addition of 80 mL ethanol, yielding 0.27 g on the 50 μm sieve, 0.69 g on the 63 μm sieve, 1.23 g on the 75 μm sieve, and 0.27 g on the 100 μm sieve of 3d as a white powder, free amino groups 0.168 mmol/g.

Preparation of Hydrogel Beads 3e Containing Free Amino Groups.

3e was prepared as described for 3d except applying a stirrer speed of 540 rpm, the use of 573 mg Cithrol™, 1000 mg 1b, 5445 mg 2k, 58.0 g DMSO, 573 mg Cithrol™ DPHS, yielding 0.34 g on the 40 μm sieve, 0.51 g on the 50 μm sieve, 0.83 g on the 63 μm sieve, and 1.16 g on the 75 μm sieve of 3e as a white powder, free amino groups 0.142 mmol/g.

Preparation of Hydrogel Beads 3f Containing Free Amino Groups.

3f was prepared as described for 3c except applying a stirrer speed of 560 rpm, the use of 398 mg 1b, 2690 mg 2n, 27.8 g DMSO, 274 mg Cithrol™ DPHS, 1.8 mL TMEDA, 2.7 mL acetic acid, yielding 0.22 g on the 50 μm sieve, 0.33 g on the 63 μm sieve, and 0.52 g on the 75 μm sieve of 3f as a white powder, free amino groups 0.152 mmol/g.

Preparation of Hydrogel Beads 3g Containing Free Amino Groups.

3g was prepared as described for 3c except applying a stirrer speed of 580 rpm, the use of 250 mg 1b, 2168 mg rac-2q, 21.8 g DMSO, 215 mg Cithrol™ DPHS, 1.1 mL TMEDA, 1.7 mL acetic acid, yielding 0.09 g on the 50 μm sieve, 0.17 g on the 63 μm sieve, and 0.54 g on the 75 μm sieve of 3g as a white powder, free amino groups 0.154 mmol/g.

Preparation of Hydrogel Beads 3h Containing Free Amino Groups.

3h was prepared as described for 3c except applying a stirrer speed of 600 rpm, the use of 250 mg 1b, 2402 mg rac-2t, 23.9 g DMSO, 235 mg Cithrol™ DPHS, 1.1 mL TMEDA, 1.7 mL acetic acid, yielding 0.27 g on the 63 μm sieve, 0.54 g on the 75 μm sieve, and 0.02 g on the 100 μm sieve of 3h as a white powder, free amino groups 0.144 mmol/g.

### Example 4

### Preparation of Maleimide Functionalized Hydrogel Beads 4

228.4 mg of dry weight hydrogel beads 3b (0.142 mmol/g amino groups/0.032 mmol amino groups) were swollen in 10 mL NMP and washed five times with NMP and five times with 2% DIEA in NMP. 108.7 mg (0.162 mmol, 5.1 eq) of Mal-PEG₆-Pfp were dissolved in NMP and added to the washed hydrogel beads 3b. The hydrogel suspension was incubated for 2 h at room temperature. Resulting maleimide functionalized hydrogel beads 4 were washed five times each with NMP and afterwards with 0.1% acetic acid, 0.01% Tween20.

Maleimide content of the hydrogel beads was determined to be 0.1204 mmol/g by conjugation of a Fmoc-cysteine to the maleimide groups on the hydrogel and subsequent Fmoc-determination as described by Gude, M., J. Ryf, et al. (2002) Letters in Peptide Science 9(4): 203-206.

### Example 5

### Synthesis of PEGylated Hydrogel Beads 5a-c

50 mg of maleimide functionalized hydrogel beads 4 (6.02×10⁻³ mmol maleimide groups) as a suspension in 0.1% acetic acid, 0.01% Tween20 were transferred into a syringe with a frit and the solvent was expelled. The hydrogel was washed ten times with PBS-T/5 mM EDTA/pH 6.5.

PEG-SH was dissolved in 0.5 mL PBS-T/5 mM EDTA/pH 6.5. The PEGsolution was drawn into the syringe and the resulting hydrogel suspension was allowed to incubate for 2.5 hours at ambient temperature under gentle shaking. The solvent was expelled and the hydrogel was washed five times with PBS-T/5 mM EDTA/pH 6.5 and transferred into a 5 mL Sarstedtvial.vial to give a hydrogel suspension of 5a.

Maleimide content on the hydrogel beads was determined via conjugation of a Fmoc-cysteine to the maleimide groups on the hydrogel and subsequent Fmoc-determination as described by Gude, M., J. Ryf, et al. (2002) Letters in Peptide Science 9(4): 203-206. Maleimide content after PEGylation was determined to be 0.0895 mmol/g. Based on equation (2) this refers to a degree of PEGylation of 17.7% of the initial maleimides can be determined.

5b was synthesized according to the procedure described for 5a, starting with 50 mg of maleimide functionalized hydrogel beads 4 (6.02×10⁻³ mmol maleimide groups) and proceeding with addition of 15.4 mg 10 kDaPEG-SH (1.54×10⁻³ mmol, 0.26 eq). Maleimide content after PEGylation was determined to be 0.0761 mmol/g. Based on equation (2) this refers to a PEGylation of 20.9% of the initial maleimides.

5c was synthesized according to the procedure described for 5a, starting with 50 mg of maleimide functionalized hydrogel beads 4 (6.02×10⁻³ mmol maleimide groups) and proceeding with addition of 26.5 mg 20 kDaPEG-SH (1.325×10⁻³ mmol, 0.22 eq). Maleimide content after PEGylation was determined to be 0.0951 mmol/g. Based on equation (2) this refers to a PEGylation of 7.2% of the initial maleimides

### Example 6

### Synthesis of PEGylated Hydrogel Beads 6a-c

75 mg of dry weight hydrogel beads 3a were transferred into a 10 mL syringe equipped with a frit and 2 NMP were added. The hydrogel was allowed to swell for 10 min at ambient temperature under gentle shaking. The solvent was expelled and the hydrogel washed 10 times with each time 3 mL 1% TMEDA/DMSO, the solvent was each time discarded. 180 mg (3×10⁻³ mmol, 0.2 eq based on amine content) branched 60 kDa PEG-NHS were dissolved in 1 mL DMSO at 37° C. and 4.5 μL of TMEDA (0.03 mmol, 3.5 mg, 2 eq. based on amine content) were added. The solution was drawn into the syringe and the resulting hydrogel suspension was allowed to incubate for 64 hours at ambient temperature under gentle shaking. The solvent was expelled and the hydrogel was washed 5 times with each time 3 mL DMSO, followed by 5 washing cycles with each time 3 mL 0.1% acetic acid/0.01% Tween 20. Fresh 0.1% acetic acid/0.01% Tween 20 was pulled into the syringe the give a suspension of 25 mg/mL hydrogel based on initial weight to give 6a. Amino content of the hydrogel beads was determined to be 0.19 mmol/g for dry weight hydrogel beads. Based on equation (2) this refers to a PEGylation of 0.4% of the initial amines.

Preparation of 6b was performed according to the procedure described for 6a except for the use of 121 mg (3×10⁻³ mmol, 0.2 eq based on amine content) branched 40 kDa PEG-NHS instead of 180 mg (3×10⁻³ mmol, 0.2 eq based on amine content) branched 60 kDa PEG-NHS resulting in an amino content of 0.191 mmol/g for dry weight hydrogel beads. Based on equation (2) this refers to a PEGylation of 0.6% of the initial amines.

Preparation of 6c was performed according to the procedure described for 6a except for the use of 60.3 mg (3×10⁻³ mmol, 0.2 eq based on amine content) 20 kDa PEG-NHS instead of 180 mg (3×10⁻³ mmol, 0.2 eq based on amine content) branched 60 kDa PEG-NHS resulting in an amino content of 0.126 mmol/g for dry weight hydrogel beads. Based on equation (2) this refers to a PEGylation of 10.6% of the initial amines.

### Example 7

### Preparation of Maleimide Functionalized Hydrogel Beads 7

117.7 mg dry hydrogel beads 3e were swollen in 5 mL NMP and washed five times with NMP and five times with 2% DIEA in NMP. 5 eq (56 mg) of Mal-PEG₆-Pfp (based on amine content of the hydrogel beads) were dissolved in 0.5 mL NMP and added to the washed hydrogel beads 3e. The hydrogel suspension was incubated for 2.5 h at room temperature. Resulting maleimide functionalized hydrogel beads were washed five times each with NMP and afterwards with 0.1% acetic acid, 0.01% Tween20.

### Example 8

### General Procedure for Preparation of PEGylated Hydrogel Beads Via Michael-Addition Reaction

Maleimide functionalized hydrogel beads as a suspension in 0.1% acetic acid, 0.01% Tween20 were transferred into a syringe with a frit and the solvent was expelled. The hydrogel was washed ten times with PBS-T/5 mM EDTA/pH 6.5.

0.2 eq PEG-SH (based on maleimide content of the hydrogel beads) was dissolved in PBS-T/5 mM EDTA/pH 6.5 (1 mL/15 mg reagent). The PEGsolution was drawn into the syringe and the resulting hydrogel suspension was allowed to incubate for 3.5 hours at ambient temperature under gentle shaking. The solvent was expelled and the hydrogel was washed five times with PBS-T/5 mM EDTA/pH 6.5 and transferred into a Sarstedt vial.

Maleimide content on the hydrogel beads was determined via conjugation of a Fmoc-cysteine to the maleimide groups on the hydrogel and subsequent Fmoc-determination as described by Gude, M., J. Ryf, et al. (2002) Letters in Peptide Science 9(4): 203-206.

Based on equation (2) degree of PEGylation can be determined.

Example 8a was prepared according to the procedure described in Example 8 starting with 50 mg (based on dry hydrogel weight) 7 using 13.3 mg 10 kDa PEG-SH. The final compound has a maleimide content of 0.0761 mmol/g.

Example 8b was prepared according to the procedure described in Example 8 starting with 50 mg (based on dry hydrogel weight) 7 using 26.6 mg 20 kDa PEG-SH. The final compound has a maleimide content of 0.0951 mmol/g.

### Example 9

### General Procedure for the Preparation of PEGylated Hydrogel Beads in DMSO

Dry hydrogel beads as e.g. described in 3a were transferred into a syringe equipped with a frit and NMP (5 mL/100 mg hydrogel beads) was added. The hydrogel was allowed to swell for 10 min at ambient temperature under gentle shaking. The solvent was expelled and the hydrogel washed ten times with each time DMSO, followed by ten washes with each time 1% TMEDA/DMSO (5 mL/100 mg hydrogel beads), the solvent was each time discarded.

0.2 eq (based on amine content of the hydrogel beads) PEG-NHS were dissolved in a solution containing 2 eq (based on amine content of the hydrogel beads) TMEDA in DMSO at 37° C. for 15 min. The solution was drawn into the syringe and the resulting hydrogel suspension was allowed to incubate under gentle shaking. The solvent was expelled and the hydrogel was washed five times with DMSO (5 mL/100 mg hydrogel beads), followed by five washing cycles with 0.1% acetic acid/0.01% Tween 20 (5 mL/100 mg hydrogel beads). Fresh 0.1% acetic acid/0.01% Tween 20 was pulled into the syringe to give a suspension of 10 mg/mL hydrogel based on initial weight. Amine content of the hydrogel beads was determined as described above. Based on equation (2) this refers to the degree of PEGylation.

Example 9a was prepared according to the general procedure in Example 9 with 80 mg hydrogel 3b and 326.4 mg 20 kDa PEG-NHS with a reaction time of 16h resulting in a PEGylated hydrogel with an amine content of 0.862 mmol/g. Based on equation (2) 0.8% of the amines have been PEGylated.

Example 9b was prepared according to the general procedure in Example 9 with 80 mg hydrogel 3b and 163.2 mg 10 kDa PEG-NHS with a reaction time of 16h resulting in a PEGylated hydrogel with an amine content of 0.756 mmol/g. Based on equation (2) 3.0% of the amines have been PEGylated.

Example 9c was prepared according to the general procedure in Example 9 with 50 mg hydrogel 3a and 200 mg branched 20 kDa PEG-NHS with a reaction time of 16h resulting in a PEGylated hydrogel with an amine content of 0.932 mmol/g. Based on equation (2) 0.4% of the amines have been PEGylated.

Example 9d was prepared according to the general procedure in Example 9 with 75 mg hydrogel 3c and 180 mg branched 60 kDa PEG-NHS with a reaction time of 64h resulting in a PEGylated hydrogel with an amine content of 0.190 mmol/g. Based on equation (2) 0.4% of the amines have been PEGylated.

Example 9e was prepared according to the general procedure in Example 9 with 75 mg hydrogel 3c and 120.6 mg branched 40 kDa PEG-NHS with a reaction time of 64h resulting in a PEGylated hydrogel with an amine content of 0.191 mmol/g. Based on equation (2) 0.6% of the amines have been PEGylated.

Example 9f was prepared according to the general procedure in Example 9 with 75 mg hydrogel 3c and 60.3 mg 20 kDa PEG-NHS with a reaction time of 64h resulting in a PEGylated hydrogel with an amine content of 0.126 mmol/g. Based on equation (2) 10.6% of the amines have been PEGylated.

Example 9g was prepared according to the general procedure in Example 9 with 25 mg hydrogel 3d and 50.3 mg branched 60 kDa PEG-NHS with a reaction time of 3h. The amine content of the PEGylated hydrogel was not determined.

Example 9h was prepared according to the general procedure in Example 9 with 25 mg hydrogel 3d and 50.3 mg branched 60 kDa PEG-NHS with a reaction time of 1h followed by addition of 33.6 mg branched 40 kDa PEG-NHS with a reaction time of 2h. The amine content of the PEGylated hydrogel was not determined.

Example 9i was prepared according to the general procedure in Example 9 with 25 mg hydrogel 3d and 50.3 mg branched 60 kDa PEG-NHS with a reaction time of 1h followed by addition of 33.6 mg branched 40 kDa PEG-NHS with a reaction time of 1h followed by addition of 16.8 mg branched 20 kDa PEG-NHS with a reaction time of 1h. The amine content of the PEGylated hydrogel was not determined.

Example 9j was prepared according to the general procedure in Example 9 with 50 mg hydrogel 3f and 30.4 mg 20 kDa PEG-NHS with a reaction time of 72h resulting in a PEGylated hydrogel with an amine content of 0.125 mmol/g. Based on equation (2) 0.8% of the amines have been PEGylated. Based on equation (2) 5.1% of the amines have been PEGylated.

Example 9k was prepared according to the general procedure in Example 9 with 50 mg hydrogel 3f and 45.6 mg 30 kDa PEG-NHS with a reaction time of 72h resulting in a PEGylated hydrogel with an amine content of 0.133 mmol/g. Based on equation (2) 2.5% of the amines have been PEGylated.

Example 9l was prepared according to the general procedure in Example 9 with 50 mg hydrogel 3f and 60.8 mg 40 kDa PEG-NHS with a reaction time of 72h resulting in a PEGylated hydrogel with an amine content of 0.141 mmol/g. Based on equation (2) 1.1% of the amines have been PEGylated.

Example 9m was prepared according to the general procedure in Example 9 with 50 mg hydrogel 3f and 121.6 mg branched 80 kDa PEG-NHS with a reaction time of 72h resulting in a PEGylated hydrogel with an amine content of 0.131 mmol/g. Based on equation (2) 1.2% of the amines have been PEGylated.

Example 9n was prepared according to the general procedure in Example 9 with 50 mg hydrogel 3f and 91.2 mg branched 60 kDa PEG-NHS with a reaction time of 72h resulting in a PEGylated hydrogel with an amine content of 0.137 mmol/g. Based on equation (2) 1.1% of the amines have been PEGylated.

Example 9o was prepared according to the general procedure in Example 9 with 50 mg hydrogel 3g and 29.6 mg 20 kDa PEG-NHS with a reaction time of 72h resulting in a PEGylated hydrogel with an amine content of 0.096 mmol/g. Based on equation (2) 12.9% of the amines have been PEGylated.

Example 9p was prepared according to the general procedure in Example 9 with 50 mg hydrogel 3g and 47.4 mg 30 kDa PEG-NHS with a reaction time of 72h resulting in a PEGylated hydrogel with an amine content of 0.098 mmol/g. Based on equation (2) 9.2% of the amines have been PEGylated.

Example 9q was prepared according to the general procedure in Example 9 with 50 mg hydrogel 3g and 59.5 mg 40 kDa PEG-NHS with a reaction time of 72h resulting in a PEGylated hydrogel with an amine content of 0.118 mmol/g. Based on equation (2) 4.1% of the amines have been PEGylated.

Example 9r was prepared according to the general procedure in Example 9 with 50 mg hydrogel 3g and 88.2 mg branched 60 kDa PEG-NHS with a reaction time of 72h resulting in a PEGylated hydrogel with an amine content of 0.121 mmol/g. Based on equation (2) 2.6% of the amines have been PEGylated.

Example 9s was prepared according to the general procedure in Example 9 with 50 mg hydrogel 3g and 128.3 mg branched branched 80 kDa PEG-NHS with a reaction time of 72h resulting in a PEGylated hydrogel with an amine content of 0.116 mmol/g. Based on equation (2) 2.4% of the amines have been PEGylated.

Example 9t was prepared according to the general procedure in Example 9 with 40 mg hydrogel 3g and 50 mg branched 40 kDa PEG-NHS with a reaction time of 72h resulting in a PEGylated hydrogel with an amine content of 0.114 mmol/g. Based on equation (2) 4.7% of the amines have been PEGylated.

Example 9u was prepared according to the general procedure in Example 9 with 40 mg hydrogel 3g and 50 mg branched 40 kDa PEG-NHS with a reaction time of 72h at 37° C. resulting in a PEGylated hydrogel with an amine content of 0.132 mmol/g. Based on equation (2) 2.3% of the amines have been PEGylated.

Example 9v was prepared according to the general procedure in Example 9 with 40 mg hydrogel 3g and 25 mg branched 40 kDa PEG-NHS with a reaction time of 24h after which an additional 25 mg branched 40 kDa PEG-NHS were added and the reaction was allowed to proceed for another 24h after which an additional 25 mg branched 40 kDa PEG-NHS were added and the reaction was allowed to proceed for another 24h resulting in a PEGylated hydrogel with an amine content of 0.119 mmol/g. Based on equation (2) 3.9% of the amines have been PEGylated.

Example 9w was prepared according to the general procedure in Example 9 with 40 mg hydrogel 3g and 25 mg branched 40 kDa PEG-NHS with a reaction time of 24h at 37° C. after which an additional 25 mg branched 40 kDa PEG-NHS were added and the reaction was allowed to proceed for another 24h at 37° C. after which an additional 25 mg branched 40 kDa PEG-NHS were added and the reaction was allowed to proceed for another 24h resulting at 37° C. in a PEGylated hydrogel with an amine content of 0.118 mmol/g. Based on equation (2) 4.1% of the amines have been PEGylated.

Example 9x was prepared according to the general procedure in Example 9 with 70 mg hydrogel 3h and 89 mg branched 40 kDa PEG-NHS with a reaction time of 48h resulting in a PEGylated hydrogel with an amine content of 0.108 mmol/g. Based on equation (2) 6.0% of the amines have been PEGylated.

Example 9y was prepared according to the general procedure in Example 9 with 70 mg hydrogel 3h and 133.6 mg branched 60 kDa PEG-NHS with a reaction time of 48h resulting in a PEGylated hydrogel with an amine content of 0.114 mmol/g. Based on equation (2) 3.6% of the amines have been PEGylated.

Example 9z was prepared according to the general procedure in Example 9 with 70 mg hydrogel 3h and 89 mg 40 kDa PEG-NHS with a reaction time of 48h resulting in a PEGylated hydrogel with an amine content of 0.085 mmol/g. Based on equation (2) 10.6% of the amines have been PEGylated.

Example 9aa was prepared according to the general procedure in Example 9 with 70 mg hydrogel 3h and 44.5 mg 20 kDa PEG-NHS with a reaction time of 48h resulting in a PEGylated hydrogel with an amine content of 0.125 mmol/g. Based on equation (2) 6.1% of the amines have been PEGylated.

### Example 10

### General Procedure for the Preparation of PEGylated Hydrogel Beads Under Aqueous Conditions

Dry hydrogel beads as e.g. described in 3a were transferred into a syringe equipped with a frit and NMP (5 mL/100 mg hydrogel beads) was added. The hydrogel was allowed to swell for 10 min at ambient temperature under gentle shaking. The solvent was expelled and the hydrogel washed ten times with 50 mM phosphate/acetonitrile pH 7.4, the solvent was discarded each time.

0.2 eq (based on amine content of the hydrogel beads) PEG-NHS were dissolved in 50 mM phosphate/acetonitrile pH 7.4. The solution was drawn into the syringe and the resulting hydrogel suspension was allowed to incubate under gentle shaking. The solvent was expelled and the hydrogel was washed five times with 50 mM phosphate/acetonitrile pH 7.4 (5 mL/100 mg hydrogel beads), followed by five washing cycles with 0.1% acetic acid/0.01% Tween 20 (5 mL/100 mg hydrogel beads). Fresh 0.1% acetic acid/0.01% Tween 20 was pulled into the syringe to give a suspension of 10 mg/mL hydrogel based on initial weight. Amine content of the hydrogel beads was determined as described above. Based on equation (2) this refers to the degree of PEGylation.

Example 10a was prepared according to the general procedure in Example 10 with 21.9 mg hydrogel 3d and 44 mg branched 60 kDa PEG-NHS with a reaction time of 20h. The amine content of the PEGylated hydrogel was not determined.

Example 10b was prepared according to the general procedure in Example 10 with 40 mg hydrogel 3g and 25 mg branched 40 kDa PEG-NHS with a reaction time of 24h after which an additional 25 mg branched 40 kDa PEG-NHS were added and the reaction was allowed to proceed for another 24h after which an additional 25 mg branched 40 kDa PEG-NHS were added and the reaction was allowed to proceed for another 24h resulting in a PEGylated hydrogel with an amine content of 0.136 mmol/g. Based on equation (2) 1.8% of the amines have been PEGylated.

### Example 11

### General Procedure for the Preparation of Maleimide Functionalized PEGylated Hydrogel Beads

PEGylated hydrogel beads as a suspension of 10 mg/mL based on initial weight of hydrogel beads prior to PEGylation were transfered into a syringe equipped with a frit. The solvent was expelled and the hydrogel washed ten times with water (5 mL/100 mg hydrogel beads), the solvent was discarded each time. The hydrogel beads were then washed ten times with NMP and five times with 2% DIEA in NMP. 5 eq of Mal-PEG₆-Pfp (based on amine content of the hydrogel beads) were dissolved in NMP (1 mL/50 mg reagent) and added to the washed hydrogel beads. The hydrogel suspension was incubated for 2 h at room temperature. Resulting maleimide functionalized hydrogel beads were washed five times each with NMP and afterwards with 0.1% acetic acid/0.01% Tween20.

Maleimide content of hydrogel beads was determined by conjugation of a Fmoc-cysteine to the maleimide groups on the hydrogel and subsequent Fmoc-determination as described by Gude, M., J. Ryf, et al. (2002) Letters in Peptide Science 9(4): 203-206.

Example 11a was prepared according to the general procedure described in Example 11 starting with 47 mg of 9d (based on dry weight 3c) using 31.7 mg Mal-PEG₆-Pfp.

Example 11b was prepared according to the general procedure described in Example 11 starting with 47 mg of 9e (based on dry weight 3c) using 31.7 mg Mal-PEG₆-Pfp.

Example 11c was prepared according to the general procedure described in Example 11 starting with 47 mg of 9f (based on dry weight 3c) using 31.7 mg Mal-PEG₆-Pfp.

Example 11d was prepared according to the general procedure described in Example 11 starting with 25 mg of 9g (based on dry weight 3d) using 13.5 mg Mal-PEG₆-Pfp.

Example 11e was prepared according to the general procedure described in Example 11 starting with 25 mg of 9h (based on dry weight 3d) using 13.5 mg Mal-PEG₆-Pfp.

Example 11f was prepared according to the general procedure described in Example 11 starting with 25 mg of 9g (based on dry weight 3d) using 13.5 mg Mal-PEG₆-Pfp.

Example 11g was prepared according to the general procedure described in Example 11 starting with 21.9 mg of 10a (based on dry weight 3d) using 13.5 mg Mal-PEG₆-Pfp.

Example 11h was prepared according to the general procedure described in Example 11 starting with 35 mg of 9j (based on dry weight 3f) using 13 mg Mal-PEG₆-Pfp.

Example 11i was prepared according to the general procedure described in Example 11 starting with 35 mg of 9k (based on dry weight 3f) using 13 mg Mal-PEG₆-Pfp.

Example 11j was prepared according to the general procedure described in Example 11 starting with 35 mg of 91 (based on dry weight 3f) using 13 mg Mal-PEG₆-Pfp.

Example 11k was prepared according to the general procedure described in Example 11 starting with 35 mg of 9m (based on dry weight 3f) using 13 mg Mal-PEG₆-Pfp.

Example 11l was prepared according to the general procedure described in Example 11 starting with 35 mg of 9n (based on dry weight 3f) using 13 mg Mal-PEG₆-Pfp.

Example 11m was prepared according to the general procedure described in Example 11 starting with 35 mg of 9o (based on dry weight 3g) using 17 mg Mal-PEG₆-Pfp.

Example 11n was prepared according to the general procedure described in Example 11 starting with 35 mg of 9p (based on dry weight 3g) using 17 mg Mal-PEG₆-Pfp.

Example 11o was prepared according to the general procedure described in Example 11 starting with 35 mg of 9q (based on dry weight 3g) using 17 mg Mal-PEG₆-Pfp.

Example 11p was prepared according to the general procedure described in Example 11 starting with 35 mg of 9r (based on dry weight 3g) using 17 mg Mal-PEG₆-Pfp.

Example 11q was prepared according to the general procedure described in Example 11 starting with 35 mg of 9s (based on dry weight 3g) using 17 mg Mal-PEG₆-Pfp.

Example 11r was prepared according to the general procedure described in Example 11 starting with 25 mg of 9t (based on dry weight 3g) using 13 mg Mal-PEG₆-Pfp.

Example 11s was prepared according to the general procedure described in Example 11 starting with 25 mg of 9u (based on dry weight 3g) using 13 mg Mal-PEG₆-Pfp.

Example 11t was prepared according to the general procedure described in Example 11 starting with 25 mg of 9v (based on dry weight 3g) using 13 mg Mal-PEG₆-Pfp.

Example 11u was prepared according to the general procedure described in Example 11 starting with 25 mg of 9w (based on dry weight 3g) using 13 mg Mal-PEG₆-Pfp.

Example 11v was prepared according to the general procedure described in Example 11 starting with 25 mg of 10b (based on dry weight 3g) using 13 mg Mal-PEG₆-Pfp.

Example 11w was prepared according to the general procedure described in Example 11 starting with 40 mg of 9x (based on dry weight 3h) using 21 mg Mal-PEG₆-Pfp.

Example 11w was prepared according to the general procedure described in Example 11 starting with 40 mg of 9x (based on dry weight 3h) using 21 mg Mal-PEG₆-Pfp.

Example 11x was prepared according to the general procedure described in Example 11 starting with 40 mg of 9y (based on dry weight 3h) using 21 mg Mal-PEG₆-Pfp.

Example 11y was prepared according to the general procedure described in Example 11 starting with 40 mg of 9z (based on dry weight 3h) using 21 mg Mal-PEG₆-Pfp.

Example 11z was prepared according to the general procedure described in Example 11 starting with 40 mg of 9aa (based on dry weight 3h) using 21 mg Mal-PEG₆-Pfp.

### Example 12

### General Procedure for the Preparation of Insulin PEGylated Hydrogel Beads

PEGylated hydrogel beads were synthesized according to Example 9. Hydrogel beads as a suspension in 0.1% acetic acid/0.01% Tween20 were then transferred in to a syringe equipped with a frit and the solvent was discarded. The hydrogel beads were washed five times with water (2 mL/10 mg hydrogel beads), five times with NMP (2 mL/10 mg hydrogel beads), five times with 2% DIEA in NMP (2 mL/10 mg hydrogel beads) and five times with DMSO (2 mL/10 mg hydrogel beads). The solvent was each time discarded. Based on amine content of the hydrogel beads, 3 eq of N-maleimido propionic acid NHS-ester were dissolvend in DMSO (750 μL DMSO/10 mg N-maleimido propionic acid NHS-ester) and the solution was drawn into the syringe and allowed to incubate under gentle shaking for 1.5 h at ambient temperature. The solvent was discarded and the hydrogel beads were washed five times with DMSO (2 mL/10 mg hydrogel beads) and 0.1% acetic acid/0.01% Tween20 (2 mL/10 mg hydrogel beads). The solvent was discarded each time. A fresh solution of 0.1% acetic acid/0.01% Tween20 was drawn into the syringe and the suspension was transferred in to a Falcon tube to give a final concentration of 10 mg/mL. Until further use, the suspension was stored at 4° C.

Linker conjugation of insulin with 6-tritylmercaptohexanoic acid NHS-ester and deprotection of the Trityl protecting group was performed according to the procedure described in WO2011/012719 example 10. Conjugation of the insulin-linker-conjugate to the maleimide functionalized hydrogel beads was performed according to the procedure described in WO2011/012719 example 11dc.

Example 12a was prepared according to the procedure described in the general procedure in example 12 starting with 10 mg hydrogel 9a (based on initial dry weight of 3b) using 4.5 mg deprotected insulin-linker-conjugate to give a PEGylated hydrogel loaded with 3.5 mg insulin-linker-conjugate.

Example 12b was prepared according to the procedure described in the general procedure in example 12 starting with 10 mg hydrogel 9b (based on initial dry weight of 3b) using 4.5 mg deprotected insulin-linker-conjugate to give a PEGylated hydrogel loaded with 3.5 mg insulin-linker-conjugate.

Example 12c was prepared according to the procedure described in the general procedure in example 12 starting with 10 mg hydrogel 9c (based on initial dry weight of 3a) using 3.9 mg deprotected insulin-linker-conjugate to give a PEGylated hydrogel loaded with 3.6 mg insulin-linker-conjugate.

### Example 13

### General Procedure for the Preparation of IL-1RA PEGylated Hydrogel Beads

A commercially available IL-1RA solution was buffer exchanged towards PBS-T/5 mM EDTA/pH 6.5.

Maleimide functionalized (PEGylated) hydrogel beads as a suspension in 0.1% HOAc/0.01% Tween20 were transferred into a syringe equipped with a frit. The solvent was discarded and the protein solution drawn into the syringe. The resulting suspension was allowed to incubate at ambient temperature under gentle shaking. The solvent was expelled and the hydrogel was was washed ten times with 1 mL of PBS-T/5 mM EDTA/pH 6.5, the solvent was discarded each time. The hydrogel was washed five times with 10 mM histidine/10 wt % α,α-trehalose/0.01% Tween20/pH 5.5, the solvent was discarded each time. The hydrogel was treated ten times×3 min with each time 1 mL 1 mM β-mercaptoethanol in 10 mM histidine/10 wt % α,α-trehalose/0.01% Tween20/pH 5.5, the solvent was discarded each time. The hydrogel was washed ten times with 2 mL 10 mM histidine/10 wt % α,α-trehalose/0.01% Tween20/pH 5.5, the solvent was discarded each time. Subsequently, the hydrogel was washed five times with 2 mL PBS-T pH 7.4, the solvent was discarded each time. 2 mL of fresh buffer were drawn into the syringe and the resulting hydrogel suspension was transferred into a Sarstedt vial. IL-1RA content within the hydrogel was calculated based on initial IL-1RA content correlated to IL-1RA content within the washing solutions, determined via A280 absorption (extinction coefficient of IL-1RA: 15.470 L/(mol x cm), molecular weight 17.260 Da) to give the protein load.

Example 13a was prepared according to the procedure described in Example 13 using 9.3 mg IL-1RA and 5.2 mg 11a (based on dry weight of 3c) resulting in a hydrogel loaded with 8.8 mg IL-1RA.

Example 13b was prepared according to the procedure described in Example 13 using 9.4 mg IL-1RA and 5.6 mg 11b (based on dry weight of 3c) resulting in a hydrogel loaded with 8.9 mg IL-1RA.

Example 13c was prepared according to the procedure described in Example 13 using 9.4 mg IL-1RA and 5.6 mg 11c (based on dry weight of 3c) resulting in a hydrogel loaded with 8.6 mg IL-1RA.

Example 13d was prepared according to the procedure described in Example 13 using 7.7 mg IL-1RA and 4 mg 11d (based on dry weight of 3d) resulting in a hydrogel loaded with 5.3 mg IL-1RA.

Example 13e was prepared according to the procedure described in Example 13 using 7.7 mg IL-1RA and 4 mg 11e (based on dry weight of 3d) resulting in a hydrogel loaded with 4.7 mg IL-1RA.

Example 13f was prepared according to the procedure described in Example 13 using 7.7 mg IL-1RA and 4 mg 11f (based on dry weight of 3d) resulting in a hydrogel loaded with 4.5 mg IL-1RA.

Example 13g was prepared according to the procedure described in Example 13 using 8.3 mg IL-1RA and 5 mg 11g (based on dry weight of 3d) resulting in a hydrogel loaded with 6.3 mg IL-1RA.

Example 13h was prepared according to the procedure described in Example 13 using 9.3 mg IL-1RA and 5 mg 8a (based on dry weight of 3e) resulting in a hydrogel loaded with 3.4 mg IL-1RA.

Example 13i was prepared according to the procedure described in Example 13 using 9.3 mg IL-1RA and 5 mg 8b (based on dry weight of 3e) resulting in a hydrogel loaded with 3.8 mg IL-1RA.

Example 13j was prepared according to the procedure described in Example 13 using 19 mg IL-1RA and 10 mg 11h (based on dry weight of 3f) resulting in a hydrogel loaded with 11.2 mg IL-1RA.

Example 13k was prepared according to the procedure described in Example 13 using 19 mg IL-1RA and 10 mg 11i (based on dry weight of 3f) resulting in a hydrogel loaded with 11.7 mg IL-1RA.

Example 13l was prepared according to the procedure described in Example 13 using 19 mg IL-1RA and 10 mg 11j (based on dry weight of 3f) resulting in a hydrogel loaded with 12.0 mg IL-1RA.

Example 13m was prepared according to the procedure described in Example 13 using 19 mg IL-1RA and 10 mg 11k (based on dry weight of 3f) resulting in a hydrogel loaded with 11.8 mg IL-1RA.

Example 13n was prepared according to the procedure described in Example 13 using 19 mg IL-1RA and 10 mg 11l (based on dry weight of 3f) resulting in a hydrogel loaded with 12.4 mg IL-1RA.

Example 13o was prepared according to the procedure described in Example 13 using 15.1 mg IL-1RA and 10 mg 11m (based on dry weight of 3g) resulting in a hydrogel loaded with 10.7 mg IL-1RA.

Example 13p was prepared according to the procedure described in Example 13 using 15.1 mg IL-1RA and 10 mg 11n (based on dry weight of 3g) resulting in a hydrogel loaded with 11.5 mg IL-1RA.

Example 13q was prepared according to the procedure described in Example 13 using 15.1 mg IL-1RA and 10 mg 11o (based on dry weight of 3g) resulting in a hydrogel loaded with 12.0 mg IL-1RA.

Example 13r was prepared according to the procedure described in Example 13 using 15.1 mg IL-1RA and 10 mg 11p (based on dry weight of 3g) resulting in a hydrogel loaded with 11.9 mg IL-1RA.

Example 13s was prepared according to the procedure described in Example 13 using 15.1 mg IL-1RA and 10 mg 11q (based on dry weight of 3g) resulting in a hydrogel loaded with 10.7 mg IL-1RA.

Example 13t was prepared according to the procedure described in Example 13 using 11.7 mg IL-1RA and 5 mg 11r (based on dry weight of 3g) resulting in a hydrogel loaded with 8.0 mg IL-1RA.

Example 13u was prepared according to the procedure described in Example 13 using 11.7 mg IL-1RA and 5 mg 11s (based on dry weight of 3g) resulting in a hydrogel loaded with 6.7 mg IL-1RA.

Example 13v was prepared according to the procedure described in Example 13 using 11.7 mg IL-1RA and 5 mg 11t (based on dry weight of 3g) resulting in a hydrogel loaded with 11.5 mg IL-1RA.

Example 13w was prepared according to the procedure described in Example 13 using 11.7 mg IL-1RA and 5 mg 11u (based on dry weight of 3g) resulting in a hydrogel loaded with 7.6 mg IL-1RA.

Example 13x was prepared according to the procedure described in Example 13 using 11.7 mg IL-1RA and 5 mg 11v (based on dry weight of 3g) resulting in a hydrogel loaded with 7.9 mg IL-1RA.

Example 13y was prepared according to the procedure described in Example 13 using 27.9 mg IL-1RA and 15 mg 11w (based on dry weight of 3h) resulting in a hydrogel loaded with 20.9 mg IL-1RA.

Example 13z was prepared according to the procedure described in Example 13 using 27.9 mg IL-1RA and 15 mg 11x (based on dry weight of 3h) resulting in a hydrogel loaded with 20.0 mg IL-1RA.

Example 13aa was prepared according to the procedure described in Example 13 using 27.9 mg IL-1RA and 15 mg 11y (based on dry weight of 3h) resulting in a hydrogel loaded with 19.4 mg IL-1RA.

Example 13bb was prepared according to the procedure described in Example 13 using 27.9 mg IL-1RA and 15 mg 11z (based on dry weight of 3h) resulting in a hydrogel loaded with 18.3 mg IL-1RA.

### Example 14

### Blocked Hydrogel Beads 14

Hydrogel beads were synthesized according to the procedure described in example 1 of WO 2011/012715 A1 and functionalized with maleimide groups according to the procedure described in Example 7. Afterwards, 10 mL of the hydrogel suspension at 67.4 mg/mL were transferred into a 20 mL syringe equipped with a frit. The solvent was expelled and the hydrogel washed 5 times with 10 mL 10 mM histidine/10 wt % α,α-trehalose/0.01% Tween20/pH 5.5. The solvent was expelled and 10 mL 100 mM β-mercaptoethanol in 10 mM histidine/10 wt % α,α-trehalose/0.01% Tween20/pH 5.5 were drawn into the syringe. The resulting suspension was allowed to incubate at ambient temperature under gentle shaking for 1 hour. The solvent was discarded and the hydrogel was washed 10 times with each time 10 mL PBS-T/pH 7.4, the solvent was discarded each time. Finally, fresh PBS-T/pH 7.4 was drawn into the syringe and the suspension transferred into a Falcon tube to give 14.

### Example 15

### Blocked Hydrogel Beads 15

Hydrogel beads were synthesized according to the procedure described in example 3h and functionalized with maleimide groups according to the procedure described in example 7. Afterwards, 4 mL of the hydrogel suspension at 10 mg/mL were transferred into a 20 mL syringe equipped with a frit. The solvent was expelled and the hydrogel washed 10 times with 5 mL 10 mM histidine/10 wt % α,α-trehalose/0.01% Tween20/pH 5.5. The solvent was expelled and 5 mL 1 mM β-mercaptoethanol in 10 mM histidine/10 wt % α,α-trehalose/0.01% Tween20/pH 5.5 were drawn into the syringe. The resulting suspension was allowed to incubate at ambient temperature under gentle shaking for 5 min. The solvent was discarded and the hydrogel treated 9 additional times with 5 mL 1 mM β-mercaptoethanol in 10 mM histidine/10 wt % α,α-trehalose/0.01% Tween20/pH 5.5. The solvent was each time discarded. The hydrogel beads were then washed 10 times with each time 5 mL 10 mM histidine/10 wt % α,α-trehalose/0.01% Tween20/pH 5.5, the solvent was discarded each time. The hydrogel beads were then washed ten times with each time 5 mL PBS-T/pH 7.4, the solvent was discarded each time. Finally, fresh PBS-T/pH 7.4 was drawn into the syringe and the suspension transferred into a Falcon tube to give 15.

### Example 16

### Antibody Binding to IL-1Ra Hydrogel Beads

20 μL of hydrogel suspensions (35 volume-%) in PBS-T buffer were mixed with 400 μL first antibody solution in PBS-T with 1% BSA (Sigma, A3059) and incubated for 1 h at 200 rpm in 1.5 mL Eppendorf tubes. For IL-1ra hydrogel beads a 1:50 dilution of antibody ab124962 (Anti-IL1RA antibody [EPR6483](ab124962)—Abcam, Cambridge, UK) was used. Hydrogel beads were sedimented through a centrifugation step at 100 g for 1 min in a tabletop centrifuge. The supernatant was removed by pipetting and care was taken not to remove any hydrogel beads. Washing of the beads was accomplished via two rounds of washing steps, which included addition of 1 mL PBS-T buffer, centrifugation at 100 g for 1 min and careful removal of the supematant by pipetting. 400 μL of the secondary antibody in PBS-T with 1% BSA (Sigma, A3059) were added to the beads and incubated for 1 h at 200 rpm. For IL-1ra hydrogel beads a 1:100 dilution of antibody sc-3750 (bovine anti-rabbit IgG-PE, Santa Cruz Biotechnology Inc., Santa Cruz, Calif. 95060 USA) was used. The supernatant was removed by pipetting and care was taken not to remove any hydrogel beads. Washing of the beads was accomplished via four rounds of washing steps, which included addition of 1 mL PBS-T buffer, centrifugation at 100 g for 1 min and careful removal of the supernatant by pipetting. The washed beads were resuspended in 200 μL PBS-T and transferred completely into black 96-well plates (black, non-binding, Art. no. 655900, Greiner bio-one GmbH, 72636 Frickenhausen, Germany). The fluorescence intensity was determined with a Tecan Infinite M200 fluorescence plate reader (Excitation 495 nm, Emission 575 nm, Number of flashes 25, Integration time 20 μs, Multiple reads per well 5×5 (Border 250 μm), Optimal gain).

Data Analysis

Determination of antibody binding to PEG-modified IL-1ra hydrogel beads was achieved in comparison with a standard curve of unmodified IL-1RA hydrogel beads. Unmodified IL-1RA hydrogel beads were always prepared under identical conditions as the PEGylated hydrogel beads except for the addition of PEGylation reagent. Unmodified IL-1RA hydrogel beads were mixed with placebo hydrogel beads (Example 14 or Example 15) in different ratios. Example 14 was used for Example 16a-16i, Example 15 was used for Example 16j-16bb. The plot (percentage of unmodified IL-1ra hydrogel beads versus fluorescence intensity) was fitted in a linear fashion. The percentage of antibody binding to PEGylated IL-1ra hydrogel beads was back-calculated according to the obtained calibration curve. Example 16a was prepared according to the procedure described in Example 16 using 13a. Example 16b was prepared according to the procedure described in Example 16 using 13b. Example 16c was prepared according to the procedure described in Example 16 using 13c. Example 16d was prepared according to the procedure described in Example 16 using 13d. Example 16e was prepared according to the procedure described in Example 16 using 13e. Example 16f was prepared according to the procedure described in Example 16 using 13f. Example 16g was prepared according to the procedure described in Example 16 using 13g. Example 16h was prepared according to the procedure described in Example 16 using 13h. Example 16i was prepared according to the procedure described in Example 16 using 13i. Example 16j was prepared according to the procedure described in Example 16 using 13j. Example 16k was prepared according to the procedure described in Example 16 using 13k. Example 16l was prepared according to the procedure described in Example 16 using 13l. Example 16m was prepared according to the procedure described in Example 16 using 13m. Example 16n was prepared according to the procedure described in Example 16 using 13n. Example 16o was prepared according to the procedure described in Example 16 using 13o. Example 16p was prepared according to the procedure described in Example 16 using 13p. Example 16q was prepared according to the procedure described in Example 16 using 13q. Example 16s was prepared according to the procedure described in Example 16 using 13s. Example 16t was prepared according to the procedure described in Example 16 using 13t. Example 16u was prepared according to the procedure described in Example 16 using 13u. Example 16v was prepared according to the procedure described in Example 16 using 13v. Example 16w was prepared according to the procedure described in Example 16 using 13w. Example 16x was prepared according to the procedure described in Example 16 using 13x. Example 16y was prepared according to the procedure described in Example 16 using 13y. Example 16z was prepared according to the procedure described in Example 16 using 13z. Example 16aa was prepared according to the procedure described in Example 16 using 13aa. Example 16bb was prepared according to the procedure described in Example 16 using 13bb.

### Example 17

### Antibody Binding to Insulin Hydrogel Beads

20 μL of hydrogel suspensions (35 volume-%) in PBS-T buffer were mixed with 400 μL first antibody solution in PBS-T with 1% BSA (Sigma, A3059) and incubated for 1 h at 200 rpm in 1.5 mL Eppendorf tubes. For Insulin hydrogel beads a 1:100 dilution of antibody ab8302 (Anti-Insulin antibody [7F8](ab8302)—Abcam, Cambridge, UK) was used. Hydrogel beads were sedimented through a centrifugation step at 100 g for 1 min in a tabletop centrifuge. The supernatant was removed by pipetting and care was taken not to remove any hydrogel beads. Washing of the beads was accomplished via two rounds of washing steps, which included addition of 1 mL PBS-T buffer, centrifugation at 100 g for 1 min and careful removal of the supernatant by pipetting. 400 μL of the secondary antibody in PBS-T with 1% BSA (Sigma, A3059) were added to the beads and incubated for 1 h at 200 rpm. For Insulin hydrogel beads a 1:50 dilution of antibody ab97041 (Goat Anti-Mouse IgG H&L (Phycoerythrin) preadsorbed (ab97041)—Abcam, Cambridge, UK) was used. The supernatant was removed by pipetting and care was taken not to remove any hydrogel beads. Washing of the beads was accomplished via four rounds of washing steps, which included addition of 1 mL PBS-T buffer, centrifugation at 100 g for 1 min and careful removal of the supernatant by pipetting. The washed beads were resuspended in 200 μL PBS-T and transferred completely into black 96-well plates (black, non-binding, Art. no. 655900, Greiner bio-one GmbH, 72636 Frickenhausen, Germany). The fluorescence intensity was determined with a Tecan Infinite M200 fluorescence plate reader (Excitation 495 nm, Emission 575 nm, Number of flashes 25, Integration time 20 μs, Multiple reads per well 5×5 (Border 250 μm), Optimal gain).

Data Analysis

Determination of antibody binding to PEG-modified Insulin hydrogel beads was achieved in comparison with a standard curve of unmodified Insulin hydrogel beads. Unmodified insulin hydrogel beads were always prepared under identical conditions as the PEGylated hydrogel beads except for the addition of PEGylation reagent. The unmodified Insulin hydrogel beads were mixed with Placebo hydrogel beads (Example 14) in different ratios. The plot (percentage of unmodified Insulin hydrogel beads versus fluorescence intensity) was fitted in a linear fashion. The percentage of antibody binding to PEGylated Insulin hydrogel beads was back-calculated according to the obtained calibration curve.

Example 17a was prepared by transferring the insulin loaded hydrogel example 12a into a 2 mL syringe equipped with a frit. The solvent was discarded and the hydrogel was washed ten times with 1 mL of PBS-T buffer pH 7.4. The buffer was discarded each time. Fresh buffer was drawn into the syringe and the suspension transferred into an Eppendorf tube. Density of the hydrogel suspension was adjusted to 35 volume-% in PBS-T. Analysis of the sample was performed as described in Example 17.

Example 17b was prepared by transferring the insulin loaded hydrogel example 12b into a 2 mL syringe equipped with a frit. The solvent was discarded and the hydrogel was washed ten times with 1 mL of PBS-T buffer pH 7.4. The buffer was discarded each time. Fresh buffer was drawn into the syringe and the suspension transferred into an Eppendorf tube. Density of the hydrogel suspension was adjusted to 35 volume-% in PBS-T. Analysis of the sample was performed as described in Example 17.

Example 17c was prepared by transferring the insulin loaded hydrogel example 12c into a 2 mL syringe equipped with a frit. The solvent was discarded and the hydrogel was washed ten times with 1 mL of PBS-T buffer pH 7.4. The buffer was discarded each time. Fresh buffer was drawn into the syringe and the suspension transferred into an Eppendorf tube. Density of the hydrogel suspension was adjusted to 35 volume-% in PBS-T. Analysis of the sample was performed as described in Example 17.

| | | | | | Final hydrogel | | |
|---|---|---|---|---|---|---|---|
| | | | Amine content | Amine | formulation | | Percentage |
| Example | | PEGylated | before | content after | Test | Example | of antibody |
| No. | Hydrogel | Hydrogel | PEGylation | PEGylation | molecule | No. | binding [%] |
| 17a | 3b | 9a | 1.020 | 0.862 | Insulin | 12a | 11 |
| 17b | 3b | 9b | 1.020 | 0.756 | Insulin | 12b | 1.3 |
| 17c | 3a | 9c | 1.003 | 0.932 | Insulin | 12c | 2.9 |
| 16a | 3c | 9d | 0.201 | 0.19 | IL-1RA | 13a | 1.6 |
| 16b | 3c | 9e | 0.201 | 0.126 | IL-1RA | 13b | 4.4 |
| 16c | 3c | 9f | 0.201 | 0.126 | IL-1RA | 13c | 0.8 |
| 16d | 3d | 9g | 0.168 | n.d. | IL-1RA | 13d | 33.5 |
| 16e | 3d | 9h | 0.168 | n.d. | IL-1RA | 13e | 2.5 |
| 16f | 3d | 9i | 0.168 | n.d. | IL-1RA | 13f | 2.7 |
| 16g | 3d | 10a | 0.168 | n.d. | IL-1RA | 13g | 17.9 |
| 16h | 3e | 8a | 0.142 | 0.076 | IL-1RA | 13h | 1.5 |
| 16i | 3e | 8b | 0.142 | 0.095 | IL-1RA | 13i | 0.7 |
| 16j | 3f | 9j | 0.152 | 0.125 | IL-1RA | 13j | 22.8 |
| 16k | 3f | 9k | 0.152 | 0.133 | IL-1RA | 13k | 9.9 |
| 16l | 3f | 9l | 0.152 | 0.141 | IL-1RA | 13l | 5.7 |
| 16m | 3f | 9m | 0.152 | 0.131 | IL-1RA | 13m | 10.4 |
| 16n | 3f | 9n | 0.152 | 0.137 | IL-1RA | 13n | 9.3 |
| 16o | 3g | 9o | 0.154 | 0.096 | IL-1RA | 13o | 35.9 |
| 16p | 3g | 9p | 0.154 | 0.098 | IL-1RA | 13p | 11.9 |
| 16q | 3g | 9q | 0.154 | 0.118 | IL-1RA | 13q | 5.4 |
| 16r | 3g | 9r | 0.145 | 0.121 | IL-1RA | 13r | 18.2 |
| 16s | 3g | 9s | 0.145 | 0.116 | IL-1RA | 13s | 13.1 |
| 16t | 3g | 9t | 0.154 | 0.114 | IL-1RA | 13t | 6.7 |
| 16u | 3g | 9u | 0.154 | 0.132 | IL-1RA | 13u | 8.7 |
| 16v | 3g | 9v | 0.154 | 0.119 | IL-1RA | 13v | 7.9 |
| 16w | 3g | 9w | 0.154 | 0.118 | IL-1RA | 13w | 4.6 |
| 16x | 3g | 10b | 0.154 | 0.136 | IL-1RA | 13x | 28.8 |
| 16y | 3h | 9x | 0.159 | 0.108 | IL-1RA | 13y | 18.1 |
| 16z | 3h | 9y | 0.159 | 0.114 | IL-1RA | 13z | 30.2 |
| 16aa | 3h | 9z | 0.159 | 0.085 | IL-1RA | 13aa | 26.1 |
| 16bb | 3h | 9aa | 0.159 | 0.125 | IL-1RA | 13bb | 39.3 |

### ABBREVIATIONS

Boc—tert. butyloxycarbonyl
BSA—bovine serum albumine
DCC—dicyclohexylcarbodiimide
DCM—dichloromethane
DIPEA—diisopropylethylamine
DMAP—4-dimethylaminopyridine
DMSO—dimethylsulfoxide
EDTA—ethylenediaminetetraacetic acid
Fmoc—fluorenylmethyloxycarbonyl
Lys—lysine
MTBE—methyl tert.butyl ether
NHS—N-hydroxysuccinimide
NMP—N-methyl-2-pyrrolidone
PBS—phosphate buffered saline
PBS-T—phosphate buffered saline, Tween20
PEG—Polyethyleneglycol
PP—polypropylene
TSTU—O—(N-Succinimidyl)-1,1,3,3-tetramethyluronium tetrafluoroborate

1. A process for the preparation of a hydrogel suitable as carrier in a hydrogel-linked prodrug comprising the steps of
(a) providing a hydrogel having groups A^{x0}, wherein groups A^{x0} represent the same or different, preferably same, functional groups;
(b) optionally covalently conjugating a spacer reagent of formula (I)
A^{x1}-SP²-A^{x2} (I),
wherein
SP² is C₁₋₅₀ alkyl, C₂₋₅₀ alkenyl or C₂₋₅₀ alkynyl, which C₁₋₅₀ alkyl, C₂₋₅₀ alkenyl and C₂₋₅₀ alkynyl is optionally interrupted by one or more group(s) selected from the group consisting of —NH—, —N(C₁₋₄alkyl)-, —O—, —S, —C(O)—, —C(O)NH, —C(O)N(C₁₋₄alkyl)-, —O—C(O)—, —S(O)—, —S(O)₂—, 4- to 7-membered heterocyclyl, phenyl and naphthyl;
A^{x1} is a functional group for reaction with A^{x0} of the hydrogel; and
A^{x2} is a functional group;
to A^{x0} of the hydrogel from step (a); and
(c) reacting the hydrogel of step (a) or step (b) with a reagent of formula (II)
A^{x3}-Z (II),
wherein
A^{x3} is a functional group; and
Z is an inert moiety having a molecular weight ranging from 10 Da to 1000 kDa;
such that at most 99 mol-% of A^{x} or A^{x2} react with A^{x3}.

2. The process of claim 1, wherein A^{x0} is selected from the group consisting of maleimide, amine (—NH₂ or —NH—), hydroxyl (—OH), thiol, carboxyl (—COOH) and activated carboxyl (—COY¹, wherein Y¹ is selected from formulas (f-i) to (f-vi): 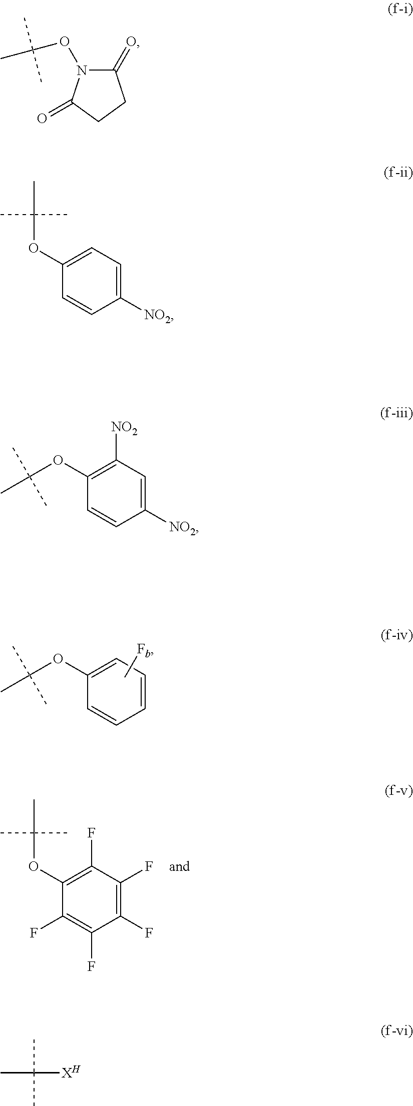
wherein
the dashed lines indicate attachment to the rest of the molecule,
b is 1, 2, 3 or 4;
X^{H} is Cl, Br, I, or F).

3. The process of claim 1 or 2, wherein the hydrogel of step (a) is obtainable by a process comprising the steps of:
(a-i) providing a mixture comprising
(a-ia) at least one backbone reagent, wherein the at least one backbone reagent has a molecular weight ranging from 1 to 100 kDa, and comprises at least three functional groups A^{x0}, wherein each A^{x0} is a maleimide, amine (—NH₂ or —NH—), hydroxyl (—OH), carboxyl (—COOH) or activated carboxyl (—COY¹, wherein Y¹ is selected from formulas (f-i) to (f-vi): 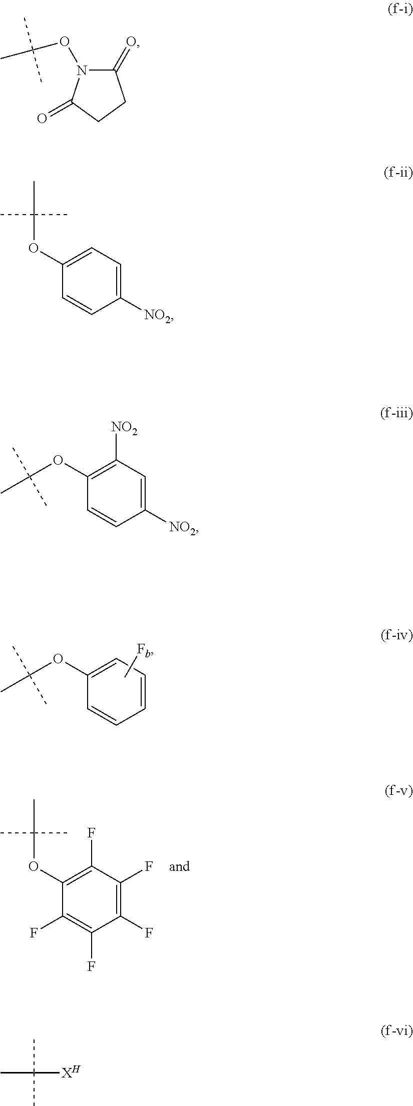
wherein
the dashed lines indicate attachment to the rest of the molecule,
b is 1, 2, 3 or 4
X^{H} is Cl, Br, I, or F);
(a-ib) at least one crosslinker reagent, wherein the at least one crosslinker reagent has a molecular weight ranging from 0.2 to 40 kDa and comprises at least two functional end groups selected from the group consisting of activated ester groups, activated carbamate groups, activated carbonate groups, activated thiocarbonate groups, amine groups and thiol groups;
in a weight ratio of the at least one backbone reagent to the at least one crosslinker reagent ranging from 1:99 to 99:1 and wherein the molar ratio of A^{x0} to functional end groups is >1;
(a-ii) polymerizing the mixture of step (a-i) to a hydrogel; and
(a-iii) optionally working-up the hydrogel of step (a-ii).

4. The process of any one of claim 1 to 3, wherein A^{x0} is an amine and A^{x1} is ClSO₂—, R¹(C═O)—, I—, Br—, Cl—, SCN—, CN—, O═C═N—, Y¹—(C═O)—, Y¹—(C═O)—NH—, or Y¹—(C═O)—O—,
wherein
R¹ is H, C₁₋₆alkyl, C₂₋₆alkenyl, C₂₋₆alkynyl, C₃₋₈cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi): 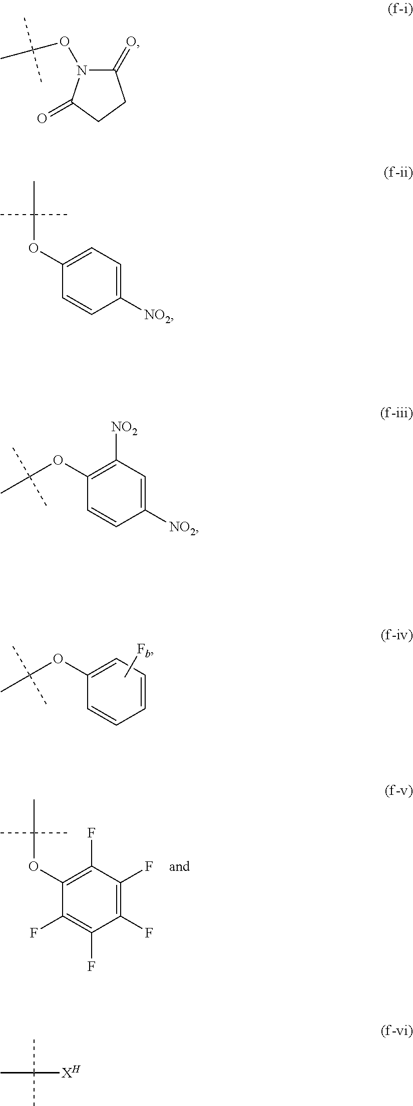
wherein
the dashed lines indicate attachment to the rest of the molecule,
b is 1, 2, 3 or 4,
X^{H} is Cl, Br, I, or F.

5. The process of any one of claims 1 to 4, wherein A^{x2} is selected from the group consisting of -maleimide, —SH, —NH₂, —SeH, —N₃, —C≡CH, —CR¹═CR^{1a}R^{1b}, —OH, —(CH═X)—R, —(C═O)—S—R¹, —(C═O)—H, —NH—NH₂, —O—NH₂, —Ar—X⁰, —Ar—Sn(R¹)(R^{1a})(R^{1b}), —Ar—B(OH)(OH), Br, I, Y¹—(C═O)—, Y¹—(C═O)—NH—, Y¹—(C═O)—O—, 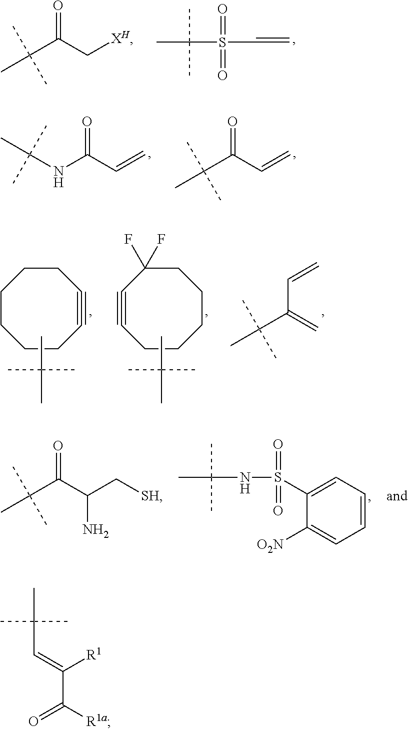
with optional protecting groups;
wherein
dashed lines indicate attachment to SP²;
X is O, S, or NH,
X⁰ is —OH, —NR¹R^{1a}, —SH, or —SeH,
X^{H} is Cl, Br, I or F;
Ar is phenyl, naphthyl, indenyl, indanyl, or tetralinyl;
R¹, R^{1a}, R^{1b} are independently of each other H, C₁₋₆alkyl, C₂₋₆alkenyl, C₂₋₆alkynyl, C₃₋₈ cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi): 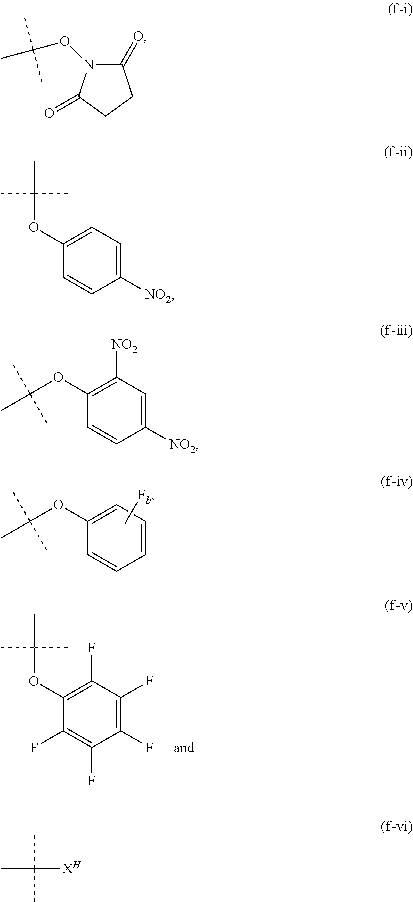
wherein
the dashed lines indicate attachment to the rest of the molecule,
b is 1, 2, 3 or 4,
X^{H} is Cl, Br, I, or F.

6. The process of any one of claims 1 to 5, wherein A^{x3} is selected from the group consisting of —SH, —NH₂, —SeH, -maleimide, —C≡CH, —N₃, —CR¹═CR^{1a}R^{1b}, —(C═X)—R¹, —OH, —(C═O)—S—R¹, —NH—NH₂, —O—NH₂, —Ar—Sn(R¹)(R^{1a})(R^{1b}), —Ar—B(OH)(OH), —Ar—X⁰, 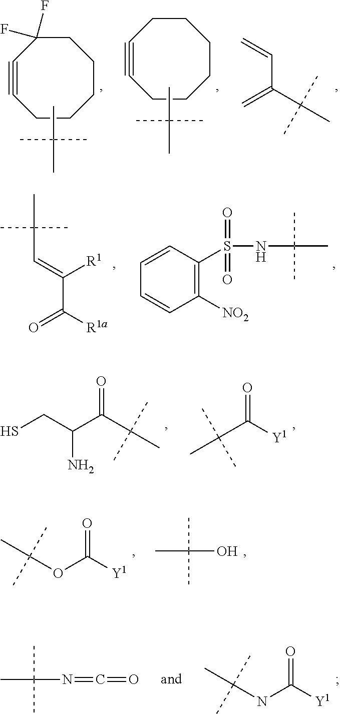
wherein
dashed lines indicate attachment to Z;
X is O, S, or NH,
X⁰ is —OH, —NR¹R^{1a}, —SH, or —SeH;
R¹, R^{1a}, R^{1b} are independently of each other H, C₁₋₆alkyl, C₂₋₆alkenyl, C₂₋₆alkynyl, C₃₋₈cycloalkyl, 4- to 7-membered heterocyclyl, 8- to 11-membered heterobicyclyl, phenyl, naphthyl, indenyl, indanyl, or tetralinyl; and
Ar is phenyl, naphthyl, indenyl, indanyl, or tetralinyl.
Y¹ is an activated carboxylic acid, activated carbonate or activated carbamate, preferably Y¹ is selected from formulas (f-i) to (f-vi): 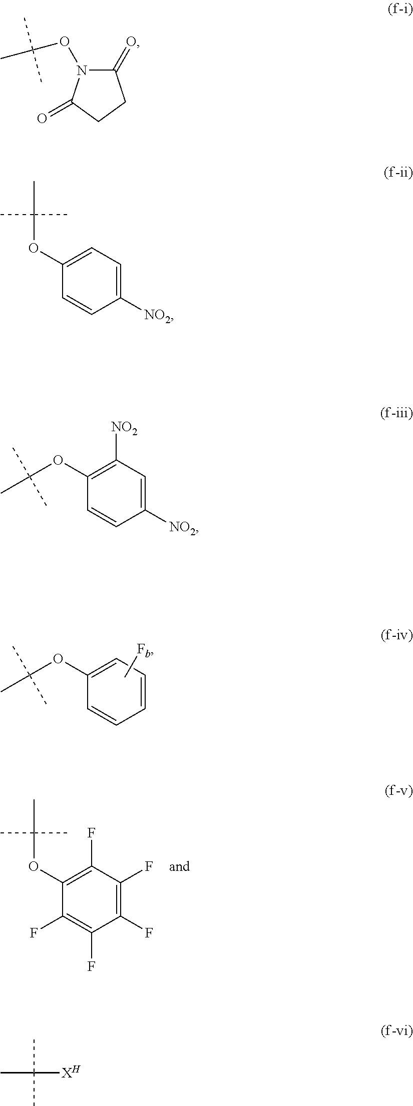
wherein
the dashed lines indicate attachment to the rest of the molecule,
b is 1, 2, 3 or 4,
X^{H} is Cl, Br, I, or F.

7. The process of any one of claims 1 to 6, wherein Z is an inert polymer having a molecular weight ranging from 0.5 kDa to 1000 kDa.

8. The process of any one of claims 1 to 7, wherein Z is an inert polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

9. The process of any one of claims 1 to 8, wherein Z is an inert linear or branched PEG-based polymer comprising at least 70% PEG.

10. The process of any one of claims 1 to 9, wherein the reagent of formula (II) is used in an amount of at most 0.99 chemical equivalents relative to A^{x0} or A^{x2}.

11. The process of any one of claims 1 to 9, wherein in step (c) the reaction rate is monitored and the reaction is interrupted when at most 0.99 chemical equivalents relative to A^{x0} or A^{x2} have reacted.

12. The process of any one of claims 1 to 9, wherein no more than 20 mol-% of A^{x0} or A^{x2} react with A^{x3}.

13. A hydrogel obtainable from the process of any one of claims 1 to 12.

14. Use of the hydrogel of claim 13 as a carrier in a hydrogel-linked prodrug.

15. A hydrogel-linked prodrug comprising a covalently conjugated hydrogel of claim 13.
